(19) United States
(12) Patent Application Publication
(10) Pub. No.: US 2025/0163002 A1
(43) Pub. Date: May 22, 2025

(54) FUSED QUINONOXIME IMIDAZOLE, ONIUM DERIVATIVES THEREOF, PREPARATION AND USE THEREOF

(71) Applicant: SHANGHAI JIAO TONG UNIVERSITY, Shanghai (CN)

(72) Inventors: Wanbin ZHANG, Shanghai (CN); Deyue YAN, Shanghai (CN); Jing YUAN, Shanghai (CN); Zhenfeng ZHANG, Shanghai (CN)

(73) Assignee: SHANGHAI JIAO TONG UNIVERSITY, Shanghai (CN)

(21) Appl. No.: 18/840,106

(22) PCT Filed: Aug. 17, 2022
(86) PCT No.: PCT/CN2022/112932

§ 371 Date: Aug. 21, 2024

(30) Foreign Application Priority Data
Mar. 7, 2022 (CN) 202210224058.9

(51) Int. Cl. C07D 235/02; A61K 31/4184; A61K 31/497; C07D 403/06; C07D 405/04; C07D 405/06; C07D 409/04; C07D 409/06

(52) U.S. Cl. CPC C07D 235/02; A61K 31/4184; A61K 31/497; C07D 403/06; C07D 405/04; C07D 405/06; C07D 409/04; C07D 409/06

(57) ABSTRACT

A fused quinonoxime imidazole, an onium derivative thereof, preparation and use thereof are provided; the fused quinonoxime imidazole and an onium derivative thereof having structures represented by the following Formulas (I), (II), (III) and (IV).

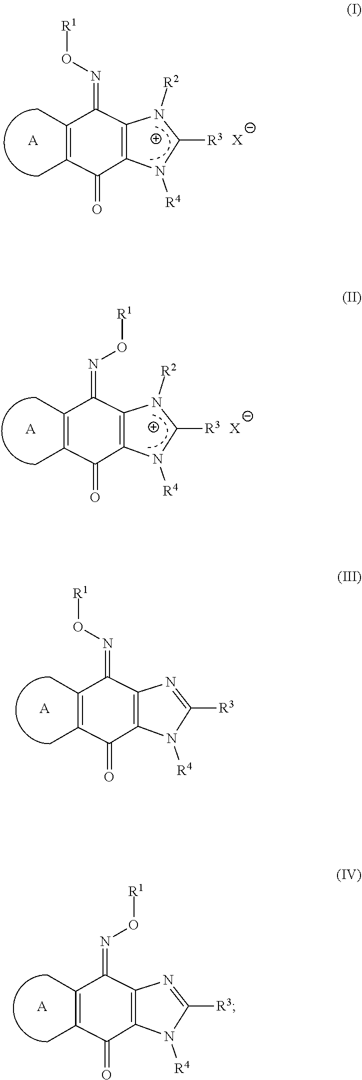

The fused quinonoxime imidazole has a novel structure, where the substituted quinone imidazole and the onium derivative thereof are fused with an aromatic carbon ring or a heterocyclic ring, and are characterized by oximation substitution. The derivative shows good inhibition activity against tumor cell growth, exhibits excellent selectivity, and has a good antitumor application prospect through in vitro and in vivo tests. Moreover, the antiviral and antibacterial effects thereof are also very good.

### CROSS REFERENCE TO THE RELATED APPLICATIONS

This application is the national phase entry of International Application No. PCT/CN2022/112932, filed on Aug. 17, 2022, which is based upon and claims priority to Chinese Patent Application No. 202210224058.9, filed on Mar. 7, 2022, the entire contents of which are incorporated herein by reference.

### TECHNICAL FIELD

The present invention falls within the field of pharmaceuticals, and relates to a fused quinonoxime imidazole, an onium derivative thereof, preparation and use thereof, and in particular to a category of antibacterial, antiviral quinonoxime imidazolium derivatives useful for the treatment of cancer and preparation thereof, newly synthesized intermediates and preparation thereof.

### BACKGROUND

Compounds containing quinones extensively exist in natural world, and many of them have good bioactivity; for example, doxorubicin and mitomycin C can be used as chemotherapeutic agents in clinical application to treat a variety of cancers; alkannin and its derivatives have anti-inflammatory, anticancer, antiviral, anti-thrombotic, glucose-lowering, liver-protecting, and other multiple biological activities. Synthesized quinone-containing drugs also have been applied widely, e.g., doxorubicin and pharmorubicin are very good front-line anticancer agents. In recent years, people have concerned studies on the antitumor activity of active substances containing quinones. N-heterocyclic carbene is a category of extensively-applied metal organic-catalyst ligands and small-molecular catalysts with excellent activity; and it is efficient, easy to get, easy to modify, cheap, etc., which is highly identical to the features of pharmaceutical molecules.

There are two categories of structure modifications on quinone imidazole derivatives reported by the existing literatures; one is accidental, unsystematic sporadic development of mono-substituted imidazole products (US patent US20090163545 A1, published on Jun. 25, 2009; international patents WO 2009023773 A2, published on Feb. 19, 2009 and WO 2012149523 A1, published on Nov. 1, 2012); another one is to synthesize 2-substituted naphthoquinone imidazole non-carbene precursor derivatives by introducing a fixed group at position 2 of naphthoquinone imidazole and then modifying the imidazole structure (Japanese patent JP 2014156400 A, published on Aug. 29, 2014; US patents US 20150086535 A1, published on Mar. 26, 2015; international US2001060803 A1 20010823, 2004092160 A1 20041028, 2012161177 A1 20121129, 2013148649 A1 20131003, and 2014142220 A1 20140918). In the present invention, quinonoids are organically combined with carbene and then subjected to oximation by fusion of subjects, which designs and synthesizes a series of quinonoxime imidazolium derivatives with efficient antitumor activity for the first time.

### SUMMARY

At present, cancer is mainly treated by chemotherapy, but toxicity of chemotherapeutic agents used clinically cannot be ignored. Therefore, there is an urgent need for developing high-activity, low-toxic, safe and reliable anti-cancer drugs. It has been found through in-depth studies on quinone drugs in the present invention that toxicity of quinones is mainly derived from ROS generated in the transformation of quinone into quinonoxime. In addition, natural quinonoxime product alkannin has good antitumor activity. The objective of the present invention is thus to provide a fused quinonoxime imidazole, an onium derivative thereof, preparation and use thereof. These compounds are novel in structure and easy to be prepared. Based on in vitro and in vivo test results, these compounds show good antitumor activity, low toxicity, safety and reliability.

The objective of the present invention is achieved by the following technical solutions:

In a first aspect, the present invention relates to a fused quinonoxime imidazolium derivative represented by the following Formula (I) and (II);

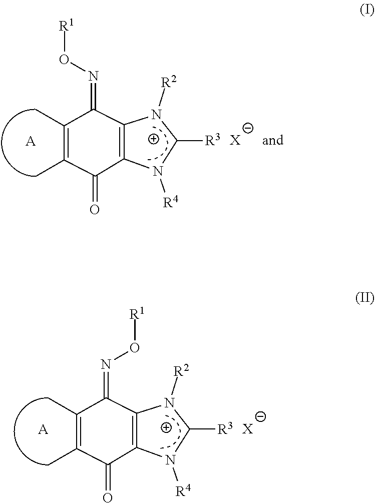

the structural formula is shown in:

In the formula, the symbols denote the following meanings:
a ring A is selected from an aromatic ring having zero, one or more substituents, or a heteroaromatic ring having zero, one or more substituents;
R¹, R², R³, and R⁴ are the same or different, and each represent-hydrogen atom, -lower linear/branched alkyl having zero, one or more substituents, -lower alkenyl having zero, one or more substituents, -lower alkynyl having zero, one or more substituents, -saturated heterocyclyl having zero, one or more substituents, -cycloalkyl having zero, one or more substituents, -cycloalkenyl having zero, one or more substituents, -aryl having zero, one or more substituents, and -heteroaryl having zero, one or more substituents;
and the substituents on the above ring A as well as in the R¹, the R², the R³, and the R⁴ are selected from a radical in group B;
group B: -lower linear/branched alkyl having zero, one or more substituents, -lower alkenyl having zero, one or more substituents, -lower alkynyl having zero, one or more substituents, -saturated heterocyclyl having zero, one or more substituents, -cycloalkyl having zero, one or more substituents, -cycloalkenyl having zero, one or more substituents, -aryl having zero, one or more substituents, -heteroaryl having zero, one or more substituents, —OR^{a}, —SR^{a}, —O-lower alkylene-OR^{a}, —O-lower alkylene-O-lower alkylene-OR^{a}, —O-lower alkylene-O-lower alkylene-O-lower alkylene-OR^{a}, —O-lower alkylene-NR^{a}R^{b}, —O-lower alkylene-O-lower alkylene-NR^{a}R^{b}, —O-lower alkylene-NR^{c}-lower alkylene-NR^{a}R^{b}, —O—CO—NR^{a}R^{b}, —SOR^{a}, —SO₂R^{a}, —SO₂NR^{a}R^{b}, —NR^{a}—SO₂R^{b}, —NR^{a}R^{b}, —NR^{c}-lower alkylene-NR^{a}R^{b}, —N(-lower alkylene-NR^{a}R^{b})₂, —NO₂, —CN, -halogen, —CO₂R^{a}, —COO—, —CONR^{a}R^{b}, —CONR^{a}—O—R^{b}, —NR^{a}—COR^{b}, —NR^{a}—CO—NR^{b}R^{c}, —OCOR^{a}, and —COR^{a};
R^{a}, R^{b}, and R^{c} are the same or different, and each represent-hydrogen atom, -lower linear/branched alkyl having zero, one or more substituents, -lower alkenyl having zero, one or more substituents, -lower alkynyl having zero, one or more substituents, -saturated heterocyclyl having zero, one or more substituents, -cycloalkyl having zero, one or more substituents, -cycloalkenyl having zero, one or more substituents, -aryl having zero, one or more substituents. -heteroaryl having zero, one or more substituents, -lower alkylene- (5 to 7-membered saturated heterocycle having zero, one or more substituents), -lower alkylene-(cycloalkyl having zero, one or more substituents), -lower alkylene-(aryl having zero, one or more substituents), and -lower alkylene-(heteroaryl having zero, one or more substituents), wherein the substituents refer to lower alkyl or heteroalkyl;
X⁻ is a coanion including a halide ion, a sulfonate ion (methanesulfonate ion, trifluoromethanesulfonate ion, phenylsulfonate ion, p-toluene sulfonate, etc.), an acetate ion, a trifluoroacetate ion, a carbonate ion, a sulfate ion, etc.; and when an anion on a substituent and an imidazolium cation form an inner salt, X⁻ is absent.

As an embodiment of the present invention, in the structural formula,
the ring A is selected from an aromatic ring having zero, one or more substituents, and the substituent is NO₂;
the R¹ is selected from -hydrogen atom, -lower linear/branched alkyl having zero, one or more substituents, -lower alkenyl having zero, one or more substituents, -lower alkynyl having zero, one or more substituents, and -aryl having zero, one or more substituents;
the substituent is selected from -cycloalkyl, -aryl, —OR^{a}, —O-lower linear/branched alkyl-OR^{a}, —O-lower linear/branched alkyl-O-lower linear/branched alkyl-OR^{a}, -aryl substituted by —OR^{a}, -aryl substituted by halogen, -aryl substituted by —CF₃, -aryl substituted by —NO₂, and —NH₂, where R^{a} is -hydrogen atom, -aryl, -lower linear/branched alkyl, or lower linear/branched alkyl substituted by aryl;
the R² is selected from lower linear/branched alkyl having zero, one or more substituents;
the R³ is selected from -hydrogen atom, -lower linear/branched alkyl having zero, one or more substituents, -cycloalkyl, -aryl, heteroaryl, -aryl substituted by lower linear/branched alkyl, -aryl substituted by —OR^{a} and/or halogen, -heteroaryl, and -heteroaryl substituted by lower linear/branched alkyl;
the substituent is -heteroaryl, -halogen, and -heteroaryl substituted by lower linear/branched alkyl; R^{a} is -lower linear/branched alkyl;
the R⁴ is selected from -hydrogen atom, -lower linear/branched alkyl having zero, one or more substituents, -lower alkenyl having zero, one or more substituents, -lower alkynyl having zero, one or more substituents, cycloalkyl, and -aryl having zero, one or more substituents;
the substituent is —CN, lower linear/branched alkyl, halogen, OR^{a}-substituted aryl, OR^{a}, heteroaryl, aryl, halogen-substituted aryl, —CO₂R^{a}, saturated heterocyclyl, and —O-lower linear/branched alkyl-OR^{a}; R^{a} is lower linear/branched alkyl.

As a further embodiment of the present invention, in the structural formula, the ring A is selected from a benzene ring, a naphthalene ring, or a NO₂-substituted benzene ring;
the R¹ is selected from -hydrogen atom, -lower alkyl, -lower alkynyl, -lower alkenyl, -lower linear/branched alkyl-O-lower linear/branched alkyl, -lower linear/branched alkyl-O-aryl, -lower linear/branched alkyl-O-lower linear/branched alkyl-O-lower linear/branched alkyl, -lower linear/branched alkyl-O-lower linear/branched alkyl-O-lower linear/branched alkyl-O-lower linear/branched alkyl, -aryl, -(5 to 7-membered) saturated heterocycle, and -heteroaryl;
the R² is selected from lower linear/branched alkyl;
the R³ is selected from -hydrogen atom, -lower linear/branched alkyl, -(3 to 7-membered) saturated cycloalkyl, -heterocyclyl, -aryl, -aryl substituted by lower linear/branched alkyl, -aryl substituted by halogen, -heteroaryl, -heteroaryl substituted by lower linear/branched alkyl, —O-lower linear/branched alkyl, —S-lower linear/branched alkyl, -acyl, and acyl substituted by lower alkyl; and
the R⁴ is selected from -hydrogen atom, -lower linear/branched alkyl, -lower alkenyl, -lower alkynyl, -aryl, —CN, -(3 to 7-membered) saturated cycloalkyl, -aryl substituted by halogen, -aryl substituted by lower alkyl, -heteroaryl, —CO₂R^{a}, -saturated heterocyclyl, -lower linear/branched alkyl-OR^{a}, -lower linear/branched alkyl-O-lower linear/branched alkyl, -lower linear/branched alkyl-S-lower linear/branched alkyl, -lower linear/branched alkyl-O-lower linear/branched alkyl-OR^{a}, where the R^{a} is -lower linear/branched alkyl.

As a further embodiment of the present invention, in the structural formula, the ring A is selected from a benzene ring, a naphthalene ring, or a NO₂-substituted benzene ring;
R¹ is selected from -hydrogen atom, —(C1 to C6) linear alkyl or branched alkyl, -benzyl, —(C4 to C6)methylcycloalkyl, —(C3 to C6)alkenyl, —CH₂ (C₆H₅₋ₘ—Xₘ)(m=1−5, X is a halogen atom, NO₂, a hydrogen atom or —OCH_{2n+1, n=1-6}), —CH₂ (C₆H₄—CHₙX₃₋ₙ)(n=1−3, X is a halogen atom), —(CₙH₂ₙ—O)_{y}—CₘH₂ₘ₊₁ (n=1−6, m=1−6, y=1−3), —CₙH₂ₙ—O—CₘH₂ₘ-Ph (n=1−6, m=1−6), —CₙH₂ₙ—NH₂ (n=1−6) or —CH₂CH(—OCH₂CH₂O—);
the R² is selected from lower linear/branched alkyl;
the R³ is selected from -hydrogen atom, —(C1 to C6) linear alkyl or branched alkyl, —(C4 to C6)methylcycloalkyl, -heteroaryl, -phenyl, or substituted phenyl;
the R⁴ is selected from —(C1 to C6) linear alkyl or branched alkyl, —(C3 to C6)methylcycloalkyl, -lower alkylene-O-lower alkyl, —(C3 to C6)alkenyl, -benzyl or substituted benzyl, an -ester group or a substituted ester group, -phenyl or substituted phenyl, —CH₂ (thienyl) or —CH₂CH(—OCH₂CH₂O—).

As a further embodiment of the present invention, in the structural formula,
the ring A is selected from a benzene ring, a naphthalene ring, or a NO₂-substituted benzene ring;
R¹ is selected from -hydrogen atom, —(C1 to C6) linear alkyl or branched alkyl, -benzyl, —(C4 to C6)methylcycloalkyl, —(C3 to C6)alkenyl, —CH₂ (C₆H₅₋ₘ—Xₘ)(m=1-5, X is a halogen atom, NO₂, a hydrogen atom or —OCₙH₂ₙ₊₁, n=1-6), —CH₂ (C₆H₄—CHₙX₃₋ₙ)(n=1-3, X is a halogen atom), —(CₙH₂ₙ—O)_{y}—CₘH₂ₘ₊₁ (n=1-6, m=1-6, y=1-3), —CₙH₂ₙ—O—CₘH₂ₘ-Ph (n=1-6, m=1-6), —CₙH₂ₙ—NH₂ (n=1-6) or —CH₂CH(—OCH₂CH₂O—);
the R² is selected from lower linear/branched alkyl;
the R³ is selected from -hydrogen atom, -methyl, -phenyl, or substituted phenyl;
the R⁴ is selected from —(C1 to C6) linear alkyl or branched alkyl; and
X is selected from Br, I, Oms, and OTs.

As a further embodiment of the present invention, the derivative includes:
(E)- or (Z)-4-(hydroxylimino)-1,3-dimethyl-9-oxo-4,9-dihydro-1H-naphtol[2,3-d]imidazole-3-onium, (E)- or (Z)-1-ethyl-4-(hydroxylimino)-3-methyl-9-oxo-4,9-dihydro-1H-naphthol[2,3-d]imidazol-3-onium, (E)- or (Z)-1-propyl-4-(hydroxylimino)-3-methyl-9-oxo-4,9-dihydro-1H-naphthol[2,3-d]imidazol-3-onium, (E)- or (Z)-1-butyl-4-(hydroxylimino)-3-methyl-9-oxo-4,9-dihydro-1H-naphthol[2,3-d]imidazole-3-onium, (E)- or (Z)-1-isopropyl-4-(hydroxylimino)-3-methyl-9-oxo-4,9-dihydro-1H-naphthol[2,3-d]imidazole-3-onium, (E)- or (Z)-1-isobutyl-4-(hydroxylimino)-3-methyl-9-oxo-4,9-dihydro-1H-naphthol[2,3-d]imidazole-3-onium, (E)- or (Z)-1-(2-methylbutyl)-4-(hydroxylimino)-3-methyl-9-oxo-4,9-dihydro-1H-naphthol[2,3-d]imidazole-3-onium, (E)- or (Z)-1-tert-butyl-4-(hydroxylimino)-3-methyl-9-oxo-4,9-dihydro-1H-naphthol[2,3-d]imidazole-3-onium, (E)- or (Z)-1-cyclopropyl-4-(hydroxylimino)-3-methyl-9-oxo-4,9-dihydro-1H-naphthol[2,3-d]imidazole-3-onium, (E)- or (Z)-1-(2-methoxyethyl)-4-(hydroxylimino)-3-methyl-9-oxo-4,9-dihydro-1H-naphthol[2,3-d]imidazole-3-onium, (E)- or (Z)-1-(3-methyl-2-ene-1-yl)-4-(hydroxylimino)-3-methyl-9-oxo-4,9-dihydro-1H-naphthol[2,3-d]imidazole-3-onium, (E)- or (Z)-1-benzyl-4-(hydroxylimino)-3-methyl-9-oxo-4,9-dihydro-1H-naphthol[2,3-d]imidazole-3-onium, (E)- or (Z)-1-(2-ethoxy-2-oxyethyl)-4-(hydroxylimino)-3-methyl-9-oxo-4,9-dihydro-1H-naphthol[2,3-d]imidazole-3-onium, (E)- or (Z)-1-phenyl-4-(hydroxylimino)-3-methyl-9-oxo-4,9-dihydro-1H-naphthol[2,3-d]imidazole-3-onium, (E)- or (Z)-1-(2,4,6-trimethylphenyl)-4-(hydroxylimino)-3-methyl-9-oxo-4,9-dihydro-1H-naphthol[2,3-d]imidazole-3-onium, (E)- or (Z)-1-(thiophene-2-methyl)-4-(hydroxylimino)-3-methyl-9-oxo-4,9-dihydro-1H-naphthol[2,3-d]imidazole-3-onium, (E)- or (Z)-1-((1,3-dioxoheterocycle-2-yl)methyl)-4-(hydroxylimino)-3-methyl-9-oxo-4,9-dihydro-1H-naphthol[2,3-d]imidazole-3-onium, (E)- or (Z)-1-(4-fluorobenzyl)-4-(hydroxylimino)-3-methyl-9-oxo-4,9-dihydro-1H-naphthol[2,3-d]imidazole-3-onium, (E)- or (Z)-1-(2-fluorophenyl)-4-(hydroxylimino)-3-methyl-9-oxo-4,9-dihydro-1H-naphthol[2,3-d]imidazole-3-onium, (E)- or (Z)-1-(3-fluorophenyl)-4-(hydroxylimino)-3-methyl-9-oxo-4,9-dihydro-1H-naphthol[2,3-d]imidazole-3-onium, (E)- or (Z)-1-(4-fluorophenyl)-4-(hydroxylimino)-3-methyl-9-oxo-4,9-dihydro-1H-naphthol[2,3-d]imidazole-3-onium, (E)- or (Z)-1-(2-fluorobenzyl)-4-(hydroxylimino)-3-methyl-9-oxo-4,9-dihydro-1H-naphthol[2,3-d]imidazole-3-onium, (E)- or (Z)-1-(3-fluorobenzyl)-4-(hydroxylimino)-3-methyl-9-oxo-4,9-dihydro-1H-naphthol[2,3-d]imidazole-3-onium, (E)- or (Z)-1-(4-methoxyphenyl)-4-(hydroxylimino)-3-methyl-9-oxo-4,9-dihydro-1H-naphthol[2,3-d]imidazole-3-onium, (E)- or (Z)-1-(4-methoxybenzyl)-4-(hydroxylimino)-3-methyl-9-oxo-4,9-dihydro-1H-naphthol[2,3-d]imidazole-3-onium, (E)- or (Z)-1-(3-chloro-4-fluorophenyl)-4-(hydroxylimino)-3-methyl-9-oxo-4,9-dihydro-1H-naphthol[2,3-d]imidazole-3-onium, (E)- or (Z)-1-(3-bromo-4-fluorophenyl)-4-(hydroxylimino)-3-methyl-9-oxo-4,9-dihydro-1H-naphthol[2,3-d]imidazole-3-onium, (E)- or (Z)-1-butyl-4-(methoxyimino)-3-methyl-9-oxo-4,9-dihydro-1H-naphthol[2,3-d]imidazolium, (E)- or (Z)-1-butyl-4-(ethoxyimino)-3-methyl-9-oxo-4,9-dihydro-1H-naphthol[2,3-d]imidazolium, (E)- or (Z)-1-butyl-4-(benzyloxyimino)-3-methyl-9-oxo-4,9-dihydro-1H-naphthol[2,3-d]imidazolium, (E)- or (Z)-1-butyl-4-(methoxyimino)-3-methyl-9-oxo-4,9-dihydro-1H-naphthol[2,3-d]imidazolium, (E)- or (Z)-1-butyl-4-(methoxyimino)-3-ethyl-9-oxo-4,9-dihydro-1H-naphthol[2,3-d]imidazolium, (E)- or (Z)-4-(methoxyimino)-1,3-dimethyl-9-oxo-4,9-dihydro-1H-naphthol[2,3-d]imidazolium, (E)- or (Z)-4-(ethoxyimino)-1,3-dimethyl-9-oxo-4,9-dihydro-1H-naphthol[2,3-d]imidazolium, (E)- or (Z)-4-(benzyloxyimino)-1,3-dimethyl-9-oxo-4,9-dihydro-1H-naphthol[2,3-d]imidazolium, (E)- or (Z)-3-ethyl-4-(methoxyimino)-1-methyl-9-oxo-4,9-dihydro-1H-naphthol[2,3-d]imidazolium, (E)- or (Z)-4-(isopropoxyimino)-1,3-dimethyl-9-oxo-4,9-dihydro-1H-naphthol[2,3-d]imidazolium, (E)- or (Z)-4-(butoxyimino)-1,3-dimethyl-9-oxo-4,9-dihydro-1H-naphthol[2,3-d]imidazolium, (E)- or (Z)-4-(isobutoxyimino)-1,3-dimethyl-9-oxo-4,9-dihydro-1H-naphthol[2,3-d]imidazolium, (E)- or (Z)-4-(tert-butoxyimino)-1,3-dimethyl-9-oxo-4,9-dihydro-1H-naphthol[2,3-d]imidazolium, (E)- or (Z)-4-(cyclopropylmethoxyimino)-1,3-dimethyl-9-oxo-4,9-dihydro-1H-naphthol[2,3-d]imidazolium, (E)- or (Z)-4-(allyloxy)imino)-1,3-dimethyl-9-oxo-4,9-dihydro-1H-naphthol[2,3-d]imidazolium, (E)- or (Z)-4-((4-methoxybenzyloxy)imino)-1,3-dimethyl-9-oxo-4,9-dihydro-1H-naphthol[2,3-d]imidazolium, (E)- or (Z)-4-((4-benzyloxy)imino)-1,3-dimethyl-9-oxo-4,9-dihydro-1H-naphthol[2,3-d]imidazolium, (E)- or (Z)-4-((4-trifluoromethylbenzyloxy)imino)-1,3-dimethyl-9-oxo-4,9-dihydro-1H-naphthol[2,3-d]imidazolium, (E)- or (Z)-4-((4-fluorobenzyloxy)imino)-1,3-dimethyl-9-oxo-4,9-dihydro-1H-naphthol[2,3-d]imidazolium, (E)- or (Z)-4-(perfluorobenzyloxyimino)-1,3-dimethyl-9-oxo-4,9-dihydro-1H-naphthol[2,3-d]imidazolium, (E)- or (Z)-4-((4-nitrobenzyloxy)imino)-1,3-dimethyl-9-oxo-4,9-dihydro-1H-naphthol[2,3-d]imidazolium, (E)- or (Z)-4-((2-methoxyethoxy)imino)-1,3-dimethyl-9-oxo-4,9-dihydro-1H-naphthol[2,3-d]imidazolium, (E)- or (Z)-4-((2-(benzyloxy) ethoxy)imino)-1,3-dimethyl-9-oxo-4,9-dihydro-1H-naphthol[2,3-d]imidazolium, (E)- or (Z)-4-((2-(2-methoxyethoxy) ethoxy)imino)-1,3-dimethyl-9-oxo-4,9-dihydro-1H-naphthol[2,3-d]imidazolium, (E)- or (Z)-4-((2-(2-(2-methoxyethoxy) ethoxy) ethoxy)imino)-1,3-dimethyl-9-oxo-4,9-dihydro-1H-naphthol[2,3-d]imidazolium, and (E)- or (Z)-4-((benzyloxy)imino)-1-butyl-3-methyl-9-oxo-2-(3,4,5-trimethoxyphenyl)-4,9-dihydro-1H-naphthol[2,3-d]imidazolium, etc.

In a second aspect, the present invention further relates to a pharmaceutical composition, including the one or more quinonoxime imidazolium derivatives above, and a pharmaceutically acceptable carrier.

In a third aspect, the present invention further relates to use of the pharmaceutical composition above in preparing an anti-cancer, anti-bacterial, or antiviral drug.

In a fourth aspect, the present invention further relates to a (Z) or (E)-fused quinonoxime imidazole derivative represented by the following Formula (I) and (II) and a hydrochloride thereof;
structural formulas are shown in:

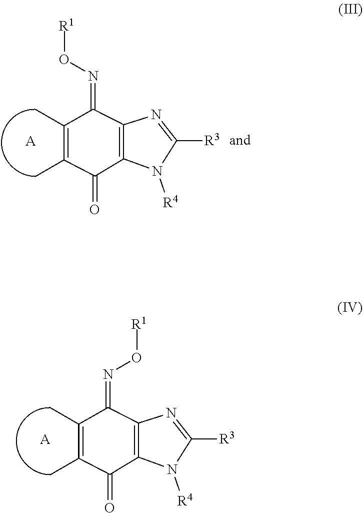

a ring A is selected from an aromatic ring having zero, one or more substituents, or a heteroaromatic ring having zero, one or more substituents;
R¹, R³, and R⁴ are the same or different, and each represent-hydrogen atom, -lower linear/branched alkyl having zero, one or more substituents, -lower alkenyl having zero, one or more substituents, -lower alkynyl having zero, one or more substituents, -saturated heterocyclyl having zero, one or more substituents, -cycloalkyl having zero, one or more substituents, -cycloalkenyl having zero, one or more substituents, -aryl having zero, one or more substituents, and -heteroaryl having zero, one or more substituents;
and the substituents on the above ring A as well as in the R¹, the R³, and the R⁴ are selected from a radical in group B;
group B: -lower linear/branched alkyl having zero, one or more substituents, -lower alkenyl having zero, one or more substituents, -lower alkynyl having zero, one or more substituents, -saturated heterocyclyl having zero, one or more substituents, -cycloalkyl having zero, one or more substituents, -cycloalkenyl having zero, one or more substituents, -aryl having zero, one or more substituents, -heteroaryl having zero, one or more substituents, —OR^{a}, —SR^{a}, —O-lower alkylene-OR^{a}, —O-lower alkylene-O-lower alkylene-OR^{a}, —O-lower alkylene-O-lower alkylene-O-lower alkylene-OR^{a}, —O-lower alkylene-NR^{a}R^{b}, —O-lower alkylene-O-lower alkylene-NR^{a}R^{b}, —O-lower alkylene-NR^{c}-lower alkylene-NR^{a}R^{b}, —O—CO—NR^{a}R^{b}, —SOR^{a}, —SO₂R^{a}, —SO₂NR^{a}R^{b}, —NR^{a}—SO₂R^{b}, —NR^{a}R^{b}, —NR^{c}-lower alkylene-NR^{a}R^{b}, —N(-lower alkylene-NR^{a}R^{b})₂, —NO₂, —CN, -halogen, —CO₂R^{a}, —COO—, —CONR^{a}R^{b}, —CONR^{a}—O—R^{b}, —NR^{a}—COR^{b}, —NR^{a}—CO—NR^{b}R^{c}, —OCOR^{a}, and —COR^{a};
R^{a}, R^{b}, and R^{c} are the same or different, and each represent-hydrogen atom, -lower linear/branched alkyl having zero, one or more substituents, -lower alkenyl having zero, one or more substituents, -lower alkynyl having zero, one or more substituents, -saturated heterocyclyl having zero, one or more substituents, -cycloalkyl having zero, one or more substituents, -cycloalkenyl having zero, one or more substituents, -aryl having zero, one or more substituents, and -heteroaryl having zero, one or more substituents; wherein the substituents refer to lower alkyl or heteroalkyl.

As an embodiment, in the structural formula,
the ring A is selected from an aromatic ring having zero, one or more substituents, and the substituent is NO₂;
the R¹ is selected from -hydrogen atom, -lower linear/branched alkyl having zero, one or more substituents, -lower alkenyl having zero, one or more substituents, and -lower alkynyl having zero, one or more substituents;
the substituent is selected from -cycloalkyl, -aryl, —OR^{a}, —O-lower linear/branched alkyl-OR^{a}, —O-lower linear/branched alkyl-O-lower linear/branched alkyl-OR^{a}, -aryl substituted by —OR^{a}, -aryl substituted by halogen, -aryl substituted by —CF₃, -aryl substituted by —NO₂, and —NH₂, where R^{a} is -hydrogen atom, -aryl, -lower linear/branched alkyl, or lower linear/branched alkyl substituted by aryl;
the R³ denotes-hydrogen atom, -lower linear/branched alkyl having zero, one or more substituents, -cycloalkyl, -aryl, heteroaryl, -aryl substituted by lower linear/branched alkyl, -aryl substituted by —OR^{a} and/or halogen, or -heteroaryl;
the substituent is -heteroaryl, -halogen, and -heteroaryl substituted by lower linear/branched alkyl; R^{a} is -lower linear/branched alkyl;
the R⁴ denotes-hydrogen atom, -lower linear/branched alkyl having zero, one or more substituents, -lower alkenyl having zero, one or more substituents, -lower alkynyl having zero, one or more substituents, cycloalkyl, or -aryl having zero, one or more substituents;
the substituent is —CN, -lower linear/branched alkyl, -halogen, -aryl substituted by OR^{a}, —OR^{a}, -heteroaryl, -aryl, -aryl substituted by halogen, —CO₂R^{a}, -saturated heterocyclyl, —O-lower linear/branched alkyl-OR^{a}; the R^{a} is -lower linear/branched alkyl.

As another embodiment, in the structural formula, the ring A is selected from an aromatic ring having zero, one or more substituents, and the substituent is NO₂;
the R¹ is selected from -hydrogen atom, -lower linear/branched alkyl having zero, one or more substituents, -lower alkenyl having zero, one or more substituents, and -lower alkynyl having zero, one or more substituents;
the substituent is selected from -cycloalkyl, -aryl, —OR^{a}, —O-lower linear/branched alkyl-OR^{a}, —O-lower linear/branched alkyl-O-lower linear/branched alkyl-OR^{a}, -aryl substituted by —OR^{a}, -aryl substituted by halogen, -aryl substituted by —CF₃, -aryl substituted by —NO₂, and —NH₂, where R^{a} is -hydrogen atom, -aryl, -lower linear/branched alkyl, or lower linear/branched alkyl substituted by aryl;
the R³ denotes-hydrogen atom, -lower linear/branched alkyl having zero, one or more substituents, -cycloalkyl, -aryl, heteroaryl, -aryl substituted by lower linear/branched alkyl, -aryl substituted by —OR^{a} and/or halogen, or -heteroaryl;
the substituent is -heteroaryl, -halogen, and -heteroaryl substituted by lower linear/branched alkyl; R^{a} is -lower linear/branched alkyl;
the R⁴ denotes-hydrogen atom, -lower linear/branched alkyl having zero, one or more substituents, -lower alkenyl having zero, one or more substituents, -lower alkynyl having zero, one or more substituents, cycloalkyl, or -aryl having zero, one or more substituents;
the substituent is —CN, -lower linear/branched alkyl, -halogen, -aryl substituted by OR^{a}, —OR^{a}, -heteroaryl, -aryl, -aryl substituted by halogen, —CO₂R^{a}, -saturated heterocyclyl, —O-lower linear/branched alkyl-OR^{a}; the R^{a} is -lower linear/branched alkyl.

As a further embodiment of the present invention, in the structural formula, the ring A is selected from a benzene ring, a naphthalene ring, or a NO₂-substituted benzene ring;
R¹ is selected from -hydrogen atom, —(C1 to C6) linear alkyl or branched alkyl, -benzyl, —(C3 to C6)methylcycloalkyl, —(C3 to C6)alkenyl, —CH₂ (C₆H₅₋ₘ—Xₘ)(X is a halogen atom, NO₂, a hydrogen atom or —OCₙH₂ₙ₊₁, n=1-6), —CH₂ (C₆H₄—CHₙX₃₋ₙ)(n=1-3, X is a halogen atom), —(CₙH₂ₙ—O)_{y}—CₘH₂ₘ₊₁ (n=1-6, m=1-6, y=1-3), —CₙH₂ₙ—O—CₘH₂ₘ-Ph (n=1-6, m=1-6) or -CH₂n-NH₂ (n=1-6);
R³ is selected from -hydrogen atom, —(C1 to C6) linear alkyl or branched alkyl, -phenyl or substituted phenyl, —(C4 to C6)methylcycloalkyl, -heteroaryl, —CHₙX₃₋ₙ (n=1-3, X is a halogen atom), or —CH₂CH₂ (2-CH₃-furyl);
R⁴ is selected from —(C1 to C6) linear alkyl or branched alkyl, —(C3 to C6)methylcycloalkyl, -lower alkylene-O-lower alkyl, —(C3 to C6)alkenyl, -alkynyl, -heteroaryl, -benzyl or substituted benzyl, an -ester group or a substituted ester group, -phenyl or substituted phenyl, -cyano, —CH₂ (thienyl), —CH₂CH(—OCH₂CH₂O—), or —CH₂ (pyrazinyl).

As a further embodiment of the present invention, in the structural formula, the ring A is selected from a benzene ring, a naphthalene ring, or a NO₂-substituted benzene ring;
R¹ is selected from -hydrogen atom, —(C1 to C6) linear alkyl or branched alkyl, -benzyl, —(C3 to C6)methylcycloalkyl, —(C3 to C6)alkenyl, —CH₂ (C₆H₅₋ₘ—Xₘ)(X is a halogen atom, NO₂, a hydrogen atom or —OCₙH₂ₙ₊₁, n=1-6), —CH₂ (C₆H₄—CHₙX₃₋ₙ)(n=1-3, X is a halogen atom), —(CₙH₂ₙ—O)_{y}—CₘH₂ₘ₊₁ (n=1-6, m=1-6, y=1-3), —CₙH₂ₙ—O—CₘH₂ₘ-Ph (n=1-6, m=1-6) or —CₙH₂ₙ—NH₂ (n=1-6);
R³ is selected from -hydrogen atom, —(C1 to C6) linear alkyl or branched alkyl;
R⁴ is selected from —(C1 to C6) linear alkyl or branched alkyl, -lower alkylene-O-lower alkyl, —(C3 to C6)alkenyl, -ester group or substituted ester group, -phenyl or substituted phenyl, and -cyano.

As a further embodiment of the present invention, the derivative includes (E)-9-(hydroxylimino)-3-methyl-3,9-dihydro-4H-naphthol[2,3-d]imidazole-4-one, (E)-3-ethyl-9-(hydroxylimino)-3,9-dihydro-4H-naphthol[2,3-d]imidazole-4-one, (E)-3-propyl-9-(hydroxylimino)-3,9-dihydro-4H-naphthol[2,3-d]imidazole-4-one, (E)-3-butyl-9-(hydroxylimino)-3,9-dihydro-4H-naphthol[2,3-d]imidazole-4-one, (E)-3-isopropyl-9-(hydroxylimino)-3,9-dihydro-4H-naphthol[2,3-d]imidazole-4-one, (E)-3-isobutyl-9-(hydroxylimino)-3,9-dihydro-4H-naphthol[2,3-d]imidazole-4-one, (E)-9-(hydroxylimino)-3-(2-methylbutyl)-3,9-dihydro-4H-naphthol[2,3-d]imidazole-4-one, (E)-9-(hydroxylimino)-3-(4-methoxybenzyl)-3,9-dihydro-4H-naphthol[2,3-d]imidazole-4-one, (E)-9-(hydroxylimino)-3-(2-methoxyethyl)-3,9-dihydro-4H-naphthol[2,3-d]imidazole-4-(E)-9-(hydroxylimino)-3-(3-methyl-2-ene-1-yl)-3,9-dihydro-4H-naphthol[2,3-d]one, imidazole-4-one, (E)-9-(hydroxylimino)-3-(propyl-2-yne-1-yl)-3,9-dihydro-4H-naphthol[2,3-d]imidazole-4-one, (E)-9-(hydroxylimino)-3-(thiophen-2-yl-methyl)-3,9-dihydro-4H-naphthol[2,3-d]imidazole-4-one, (E)-9-(hydroxylimino)-3-benzyl-3,9-dihydro-4H-naphthol[2,3-d]imidazole-4-one, (E)-9-(hydroxylimino)-3-(2-fluorobenzyl)-3,9-dihydro-4H-naphthol[2,3-d]imidazole-4-one, (E)-9-(hydroxylimino)-3-(3-fluorobenzyl)-3,9-dihydro-4H-naphthol[2,3-d]imidazole-4-one, (E)-9-(hydroxylimino)-3-(4-fluorobenzyl)-3,9-dihydro-4H-naphthol[2,3-d]imidazole-4-one, (E)-2-(4-hydroxylimino)-9-oxo-4,9-dihydro-1H-naphthol[2,3-d]imidazole-1-yl)acetate, (E)-3-((1,3-dioxoheterocycle-2-yl)methyl)-9-(hydroxylimino)-3,9-dihydro-4H-naphthol[2,3-d]imidazole-4-one, (E)-11-(hydroxylimino)-3-methyl-3,11-dihydro-4H-anthraquinone[2,3-d]imidazole-4-one, (E)-11-(hydroxylimino)-3-ethyl-3,11-dihydro-4H-anthraquinone[2,3-d]imidazole-4-one, (E)-3-cyclopropyl-9-(hydroxylimino)-3,9-dihydro-4H-naphthol[2,3-d]imidazole-4-one, (E)-3-tert-butyl-9-(hydroxylimino)-3,9-dihydro-4H-naphthol[2,3-d]imidazole-4-one, (E)-9-(hydroxylimino)-3-(pyrazine-2-yl-methyl)-3,9-dihydro-4H-naphthol[2,3-d]imidazole-4-one, (E)-9-(hydroxylimino)-3-benzyl-3,9-dihydro-4H-naphthol[2,3-d]imidazole-4-one, (E)-9-(hydroxylimino)-3-(2,4,6-trimethylphenyl)-3,9-dihydro-4H-naphthol[2,3-d]imidazole-4-one, (E)-9-(hydroxylimino)-3-(2-fluorophenyl)-3,9-dihydro-4H-naphthol[2,3-d]imidazole-4-one, (E)-9-(hydroxylimino)-3-(3-fluorophenyl)-3,9-dihydro-4H-naphthol[2,3-d]imidazole-4-one, (E)-9-(hydroxylimino)-3-(4-fluorophenyl)-3,9-dihydro-4H-naphthol[2,3-d]imidazole-4-one, (E)-9-(hydroxylimino)-3-(4-methoxyphenyl)-3,9-dihydro-4H-naphthol[2,3-d]imidazole-4-one, (E)-9-(hydroxylimino)-3-(3-chloro-4-fluorophenyl)-3,9-dihydro-4H-naphthol[2,3-d]imidazole-4-one, (E)-9-(hydroxylimino)-3-(3-bromo-4-fluorophenyl)-3,9-dihydro-4H-naphthol[2,3-d]imidazole-4-one, (E)-9-(hydroxylimino)-3-methyl-8-nitro-3,9-dihydro-4H-naphthol[2,3-d]imidazole-4-one, (E)-9-(hydroxylimino)-2,3-dimethyl-3,9-dihydro-4H-naphthol[2,3-d]imidazole-4-one, (E)-3-butyl-9-(hydroxylimino)-2-methyl-3,9-dihydro-4H-naphthol[2,3-d]imidazole-4-one, (E)-11-(hydroxylimino)-2,3-dimethyl-3,11-dihydro-4H-anthraquinone[2,3-d]imidazole-4-one, (E)-3-ethyl-11-(hydroxylimino)-2-methyl-3,11-dihydro-4H-anthraquinone[2,3-d]imidazole-4-one, (E)-3-isobutyl-11-(hydroxylimino)-2-methyl-3,11-dihydro-4H-anthraquinone[2,3-d]imidazole-4-one, (E)-11-(hydroxylimino)-2-methyl-3-(3-methyl-2-ene-1-yl)-3,11-dihydro-4H-anthraquinone[2,3-d]imidazole-4-one, (E)-2-(4-(hydroxylimino)-2-methyl-11-oxo-4,11-dihydro-1H-anthracene[2,3-d]imidazole-1-yl) acetonitrile, (E)-3-benzyl-11-(hydroxylimino)-2-methyl-3,11-dihydro-4H-anthraquinone[2,3-d]imidazole-4-one, (E)-3-(4-fluorobenzyl)-11-(hydroxylimino)-2-methyl-3,11-dihydro-4H-anthraquinone[2,3-d]imidazole-4-one, (E)-3-butyl-9-(hydroxylimino)-2-ethyl-3,9-dihydro-4H-naphthol[2,3-d]imidazole-4-one, (E)-3-butyl-9-(hydroxylimino)-2-propyl-3,9-dihydro-4H-naphthol[2,3-d]imidazole-4-one, (E)-2-(sec-butyl)-3-butyl-9-(hydroxylimino)-3,9-dihydro-4H-naphthol[2,3-d]imidazole-4-one, (E)-3-butyl-9-(hydroxylimino)-2-isobutyl-3,9-dihydro-4H-naphthol[2,3-d]imidazole-4-one, (E)-3-butyl-9-(hydroxylimino)-2-(pentane-3-yl)-3,9-dihydro-4H-naphthol[2,3-d]imidazole-4-one, (E)-3-butyl-9-(hydroxylimino)-2-tert-butyl-3,9-dihydro-4H-naphthol[2,3-d]imidazole-4-one, (E)-3-butyl-9-(hydroxylimino)-2-(2-(5-methylfuran-2-yl)-ethyl)-3,9-dihydro-4H-naphthol[2,3-d]imidazole-4-one, (E)-3-butyl-9-(hydroxylimino)-2-cyclobutyl-3,9-dihydro-4H-naphthol[2,3-d]imidazole-4-one, (E)-3-butyl-9-(hydroxylimino)-2-phenyl-3,9-dihydro-4H-naphthol[2,3-d]imidazole-4-one, (E)-3-butyl-9-(hydroxylimino)-2-(4-isopropylphenyl)-3,9-dihydro-4H-naphthol[2,3-d]imidazole-4-one, (E)-3-butyl-9-(hydroxylimino)-2-(3,4,5-trimethoxyphenyl)-3,9-dihydro-4H-naphthol[2,3-d]imidazole-4-one, (E)-3-butyl-2-(3-chloro-4-methoxyphenyl)-9-(hydroxylimino)-3,9-dihydro-4H-naphthol[2,3-d]imidazole-4-one, (E)-3-butyl-9-(hydroxylimino)-2-naphthyl-3,9-dihydro-4H-naphthol[2,3-d]imidazole-4-one, (E)-3-butyl-9-(hydroxylimino)-2-(trifluoromethyl)-3,9-dihydro-4H-naphthol[2,3-d]imidazole-4-one, (E)-3-butyl-9-(hydroxylimino)-2-(furan-2-yl)-3,9-dihydro-4H-naphthol[2,3-d]imidazole-4-one, (E)-3-butyl-9-(hydroxylimino)-2-(thiophene-2-yl)-3,9-dihydro-4H-naphthol[2,3-d]imidazole-4-one, (E)-2-ethyl-11-(hydroxylimino)-3-methyl-3,11-dihydro-4H-anthraquinone[2,3-d]imidazole-4-one, (E)-2-(furan-2-yl)-11-(hydroxylimino)-3-methyl-3,11-dihydro-4H-anthraquinone[2,3-d]imidazole-4-one, (E) and (Z)-9-(methoxyimino)-3-methyl-3,9-dihydro-4H-naphthol[2,3-d]imidazole-4-one, (E) and (Z)-9-(ethoxyimino)-3-methyl-3,9-dihydro-4H-naphthol[2,3-d]imidazole-4-one, (E) and (Z)-9-(benzyloxyimino)-3-methyl-3,9-dihydro-4H-naphthol[2,3-d]imidazole-4-one, (E) and (Z)-9-(isopropoxyimino)-3-methyl-3,9-dihydro-4H-naphthol[2,3-d]imidazole-4-one, (E) and (Z)-9-(butoxyimino)-3-methyl-3,9-dihydro-4H-naphthol[2,3-d]imidazole-4-one, (E) and (Z)-9-(isobutoxyimino)-3-methyl-3,9-dihydro-4H-naphthol[2,3-d]imidazole-4-one, (E) and (Z)-9-(tert-butoxyimino)-3-methyl-3,9-dihydro-4H-naphthol[2,3-d]imidazole-4-one, (E) and (Z)-9-((cyclopropoxy)imino)-3-methyl-3,9-dihydro-4H-naphthol[2,3-d]imidazole-4-one, (E) and (Z)-9-((allyloxy)imino)-3-methyl-3,9-dihydro-4H-naphthol[2,3-d]imidazole-4-one, (E) and (Z)-3-methyl-9-(((4-methoxybenzyl)oxy)imino)-3,9-dihydro-4H-naphthol[2,3-d]imidazole-4-one, (E) and (Z)-3-methyl-9-(((4-chlorobenzyl)oxy)imino)-3,9-dihydro-4H-naphthol[2,3-d]imidazole-4-one, (E) and (Z)-3-methyl-9-(((4-trifluoromethylbenzyl)oxy)imino)-3,9-dihydro-4H-naphthol[2,3-d]imidazole-4-one, (E) and (Z)-3-methyl-9-(((4-fluorobenzyl)oxy)imino)-3,9-dihydro-4H-naphthol[2,3-d]imidazole-4-one, (E) and (Z)-3-methyl-9-(((perfluorobenzyl)oxy)imino)-3,9-dihydro-4H-naphthol[2,3-d]imidazole-4-one, (E) and (Z)-3-methyl-9-(((4-nitrobenzyl)oxy)imino)-3,9-dihydro-4H-naphthol[2,3-d]imidazole-4-one, (E) and (Z)-11-(benzyloxyimino)-3-methyl-3,11-dihydro-4H-anthraquinone[2,3-d]imidazole-4-one, (E) and (Z)-11-(butoxyimino)-3-methyl-3,11-dihydro-4H-anthraquinone[2,3-d]imidazole-4-one, (E) and (Z)-11-(tert-butoxyimino)-3-methyl-3,11-dihydro-4H-anthraquinone[2,3-d]imidazole-4-one, (E)-9-((2-methoxyethoxy)imino)-3-methyl-3,9-dihydro-4H-naphthol[2,3-d]imidazole-4-one, (E) and (Z)-9-((2-benzyloxy) ethoxy)imino)-3-methyl-3,9-dihydro-4H-naphthol[2,3-d]imidazole-4-one, (E)-9-((2-(2-methoxyethoxy) ethoxy)imino)-3-methyl-3,9-dihydro-4H-naphthol[2,3-d]imidazole-4-one, (E) and (Z)-9-((2-(2-(2-methoxyethoxy) ethoxy) ethoxy)imino)-3-methyl-3,9-dihydro-4H-naphthol[2,3-d]imidazole-4-one, (E)-9-((3-aminopropoxy)imino)-3-methyl-3,9-dihydro-4H-naphthol[2,3-d]imidazole-4-one, (E) and (Z)-3-butyl-9-(methoxyimino)-3,9-dihydro-4H-naphthol[2,3-d]imidazole-4-one, (E) and (Z)-3-butyl-9-(ethoxyimino)-3,9-dihydro-4H-naphthol[2,3-d]imidazole-4-one, (E) and (Z)-9-((benzyloxy)imino)-3-butyl-3,9-dihydro-4H-naphthol[2,3-d]imidazole-4-one, (E) and (Z)-11-((methoxy)imino)-3-(2-(2-methoxyethoxy)ethyl)-3, 11-dihydro-4H-anthraquinone[2,3-d]imidazole-4-one, (E) and (Z)-9-((benzyloxy)imino)-2,3-dimethyl-3,9-dihydro-4H-naphthol[2,3-d]imidazole-4-one, (E) and (Z)-11-((benzyloxy)imino)-2,3-dimethyl-3,11-dihydro-4H-anthraquinone[2,3-d]imidazole-4-one, (E) and (Z)-9-((benzyloxy)imino)-3-butyl-2-(3,4,5-trimethoxyphenyl)-3,9-dihydro-4H-naphthol[2,3-d]imidazole-4-one, and (E) and (Z)-2-cyclopropyl-11-(methoxyimino)-3-methyl-3,11-dihydro-4H-anthraquinone[2,3-d]imidazole-4-one.

The (Z) or (E)-fused quinonoxime imidazole derivative represented by Formula (III) or (IV) and a hydrochloride thereof are synthetic intermediates of the above Formulas (I) and (II); and the synthetic intermediates have good antitumor activity. Therefore, in a fifth aspect, the present invention further relates to a pharmaceutical composition, including the compound or hydrochloride thereof, and a pharmaceutically acceptable carrier. The pharmaceutical composition has anticancer, antibacterial and antiviral effects.

In a fifth aspect, the present invention further relates to use of the above pharmaceutical composition above in preparing an anti-cancer, anti-bacterial, or antiviral drug.

Compounds of Formulas (I), (II), (III), and (IV) will be further described below.

Term “lower” refers to a linear or branched hydrocarbon having 1 to 6 carbon atoms. “Lower alkyl” generally refers to alkyl having 1 to 4 carbon atoms, particularly preferably, methyl, ethyl, propyl, isopropyl, butyl, and isobutyl. “Lower alkenyl” generally refers to vinyl, allyl, 1-propenyl, isopropenyl, 1-butenyl, 2-butenyl, 3-butenyl, and 3-methyl-2-butenyl. “Lower alkynyl” generally refers to acetenyl, 1-propinyl, 2-propinyl, 1-butynyl, 2-butynyl, 3-butynyl, and 1-methyl-2-propinyl. “Aryl” refers to aromatic radicals, more preferably, aryl having 6 and 14 carbon atoms, and most preferably, phenyl, naphthyl, and fluorenyl. In addition, ring A refers to a ring forming the above aryl, most preferably, phenyl and naphthyl. “Heteroaromatic ring” refers to 5-6 membered monocyclic heteroaryl having 1-4 heteroatoms selected from one or more of N, S, and O, and bicyclic heteroaryl obtained by fusing a benzene ring or 5-6 membered monocyclic heteroaryl therewith. In this case, the 5-6 membered monocyclic heteroaryl is preferably furyl, thienyl, pyrryl, imidazolyl, thiazolyl, pyrazolyl, isothiazolyl, oxazolyl, isoxazolyl, oxadiazolyl, thiadiazolyl, triazolyl, tetrazolyl, pyridyl, pyrimidyl, pyrazinyl, pyridazinyl and triazinyl; the bicyclic heteroaryl is preferably benzofuryl, benzothiophenyl, benzothiadiazolyl, benzothiazolyl, benzoxazolyl, benzoxadiazolyl, benzimidazolyl, indolyl, isoindolyl, indazolyl, quinolyl, isoquinolyl, quinazolinyl, quinoxalinyl, benzodioxoly, indolizinyl, imidazolpyridyl, and cinnolinyl. Some of the saturated heteroaryl groups are, for example, 1,2,3,4-tetrahydroquinolyl, preferably, furyl and thienyl.

“Lower heteroalkyl” refers to C₁-C₄ alkyl having 1 to 4 heteroatoms selected from one or more of N, S, and O.

“Cycloalkyl” refers to cycloalkyl group having 3 to 10 carbon atoms, particularly preferably, cyclopropyl, cyclobutyl, cyclopentyl, and cyclohexyl. “Cycloalkenyl” is preferably cycloalkenyl having 3 to 8 carbon atoms.

“Coanion” is not limited particularly as long as it is a pharmaceutically acceptable anion as a coanion against imidazolium cation, preferably, univalent or bivalent anion, e.g., halide ions, organic sulfonate ions, acetate ions, trifluoroacetate ions, carbonate ions, and sulfate ions, most preferably, halide ions.

“Heterocyclyl” refers to 5 to 7-membered monocyclic saturated heterocycle having 1-4 heteroatoms selected from N, S, and O, or a bridge ring thereof, preferably, tetrahydrofuryl, tetrahydrofuryl, pyrrolidyl, piperazinyl, azacycloheptyl, diazacycloheptyl, quinuclidinyl, piperidyl, and morpholinyl.

The compound of the present invention has tautomers of the following formula due to the non-localized effect of cation, and the present invention also includes isolated tautomers or a mixture thereof. Therefore, in the present invention, the compound represented by 1H-imidazole-3-onium derivative also includes a mixture of the tautomer 3H-imidazole-1-onium derivative and two isomers.

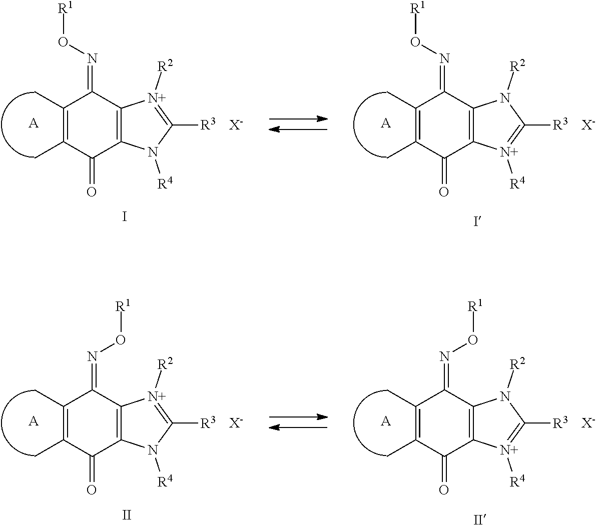

Additionally, hydrochlorides of Formulas (III) and (IV) in the present invention also fall within the present invention.

In some cases, the compounds (I), (II), (III), and (IV) of the present invention have geometrical isomers and tautomers according to different types of substituents. Isolated forms of these isomers or a mixture thereof are included in the present invention. Moreover, due to the existence of asymmetric carbon atoms in the compounds of the present invention, there are isomers to these asymmetric carbon atoms; the present invention includes mixed and isolated forms of these optical isomers. Moreover, in some cases, the compounds of the present invention form N-oxides due to different types of substituents; these substances are also included in the present invention; the present invention further includes various hydrates, solvates, and polymorphic substances of the compounds (I), (II), (III), and (IV) herein.

In a sixth aspect, the present invention further relates to a method for preparing the preceding quinonoxime imidazolium derivative and synthetic intermediates thereof; the method includes the following steps:

A, serving a protic solvent as a solvent, and subjecting a substituted quinone imidazole derivative

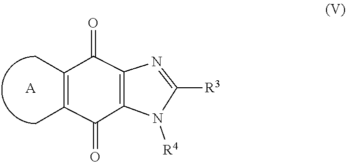

to an oximation reaction with R¹ONH₂·HCl under the catalytic action of a weak base or with corresponding R¹ONH₂ under the action of a weak-base hydrochloride, to obtain quinonoxime imidazole derivatives

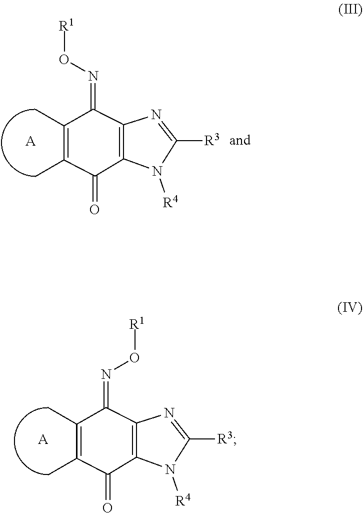

and

B, performing N-alkylation reaction on the quinonoxime imidazole derivative (III) or (IV) and a corresponding halide reagent R²X in an organic solvent, to obtain a quinonoxime imidazolium derivative (I) or (II).

Preferably, in step A, a molar ratio of the substituted naphthoquinone imidazole to the substituted oxamine hydrochloride R¹ONH₂·HCl, and to the weak base is 1:(1.2-5):(0.001-0.01).

Preferably, the substituted oxamine hydrochloride is selected from hydroxylamine hydrochloride, methoxyamine hydrochloride, phenoxyamine hydrochloride, and benzyloxyamine hydrochloride.

As an embodiment, in step A, the weak base is selected from potassium carbonate, triethylamine, pyridine, etc.

As an embodiment, in step A, the protic solvent is selected from methanol, ethanol, and isopropanol.

As an embodiment, in step A, reaction temperature is 90-125° C., and reaction time is 12-48 h.

As an embodiment, in step B, a molar ratio of the quinonoxime imidazole derivative (III) or (IV) to the halide reagent R²X is (1:1)-(1:100).

As an embodiment, in step B, the halohydrocarbon is selected from iodomethane, iodoethane, etc.

As an embodiment, in step B, the organic solvent is selected from acetonitrile, ethyl acetate, tetrahydrofuran, etc.

As an embodiment, in step B, the reaction temperature is 50-120° C., and reaction time is 8-48 h.

The compounds (III) and (IV) of the present invention may be used as a broad-spectrum anticancer, antibacterial and antiviral drug, and has low toxicity and good safety. Therefore, the compounds of the present invention have inhibition activity against proliferation of all solid tumors and lymphomas, in particular to breast cancer, cervical cancer, non-small cell lung cancer, rectal cancer, liver cancer, leukemia, prostate cancer, ovarian cancer, myeloma, esophageal cancer, pancreatic cancer, bladder cancer, melanoma, stomach cancer and other tumors.

The pharmaceutical composition of the present invention may be prepared by one or two compounds of Formulas (III) and (IV) above, and a conventional pharmaceutically acceptable carrier (a drug carrier, an excipient, etc.) in the art according to a conventional method. Administration may be conducted via any one of oral means of troches, pills, capsules, granules, powders, liquids, inhalants, etc. and para-oral means such as intravenous injection and intramuscular injection of injections, suppositories, eye drops, eye ointments, percutaneous liquids, ointments, percutaneous patches, transmucosal liquids, transmucosal patches, etc.

As solid compositions for oral administration of the present invention, troches, powders, granules, etc. are available. In these solid compositions, one or more active substances are mixed with at least one of inert excipients (e.g., lactose, mannitol, glucose, hydroxypropyl cellulose, microcrystalline cellulose, starch, polyvinylpyrrolidone, aluminum/magnesium metasilicate, etc.). According to conventional steps, the compound may contain inert additives, e.g., lubricants (magnesium stearate, etc.), disintegrating agents (sodium carboxymethyl starch, etc.), and cosolvents. According to the need, troches or pills may be coated with icing, gastric-soluble or enteric soluble coating.

Liquid compositions for oral administration include pharmaceutically acceptable emulsions, liquids, suspending agents, syrups, elixirs, etc., and contain common inert solvents such as purified water or ethanol. Besides inert solvents, the composition may further contain a solubilizer, a wetting agent, a suspending agent, a sweetener, a flavoring agent, an aromatic, a preservative, and other additives.

Injections for para-oral administration contain sterile aqueous or aqueous solvents, suspending agents, and emulsions. Examples of aqueous solvents include distilled water and normal saline for injection. Insoluble agents include propylene glycol, polyethylene glycol, olive oil and other vegetable oil, ethanol and other alcohols, polysorbate 80, etc. These compositions may further contain isotonic agents, preservatives, wetting agents, emulsifiers, dispersants, stabilizers and co-solvents. These compositions are sterilized by, for example, filtering with a sterilizing membrane, mixing with a bactericide, or irradiation, etc. In addition, it may be prepared into sterile solid compositions may be further prepared, dissolved with sterile water or sterile solvent for injection or suspended for use.

### DETAILED DESCRIPTION OF THE EMBODIMENTS

The following examples will help those skilled in the art further understand the present invention, but are not construed as limiting the present invention. It should be indicated that those skilled in the art may make further adjustments and improvements in the premise of not departing from the inventive concept herein. These all fall within the protection scope of the present invention. Compounds of the present invention are not limited to the compounds recited in the following examples at all. Experimental methods not marked with specific conditions in the following examples are generally subjected to conventional conditions, or conditions recommended by manufacturers. Moreover, the examples show the synthesis of raw materials of the compounds of the present invention.

### Example 1

2,3-Diamine-1,4-benzoquinone (20 g) was dissolved into 100 mL of formic acid, subjected to reflux reaction for 6 h, and cooled to room temperature, then poured into an ice-water mixture with the addition of ammonium hydroxide to regulate pH to 9-10, and the obtained solution was subjected to suction filtration and vacuum dried to obtain yellow solid 1,4-naphthoquinone imidazole (22 g). 1,4-naphthoquinone imidazole (0.3 g) and potassium hydroxide (0.15 g) were added to dimethyl sulfoxide solution (3 mL); the mixture was stirred at 50° C. for 30 min and added with iodomethane (0.2 mL), after reaction for 2, water was added to separate out solid; the solid was subjected to suction filtration, washed with water, and vacuum dried to obtain yellow solid 2-methyl-1,4-naphthoquinone imidazole (0.31 g). 2-methyl-naphthoquinone imidazole (0.21 g), hydroxylamine hydrochloride (0.35 g), and pyridine (0.8 mL) were dissolved into ethanol (70 mL) and reacted for 48 h at 90° C.; at the end of reaction, solid was separated out from solution, directly subjected to suction filtration, and vacuum dried to obtain light yellow solid (0.15 g), i.e., Compound 1.

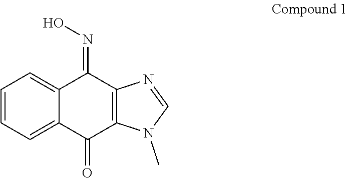 [SMILES: Cn1cnc2c1C(=O)c1ccccc1/C2=N\O]

(E)-9-(hydroxylimino)-3-methyl-3,9-dihydro-4H-naphthol[2,3-d]imidazole-4-one

¹H NMR (400 MHZ, DMSO-d₆) δ 13.01 (s, 1H), 8.26 (d, J=0.8 Hz, 2H), 8.07 (dd, J=8.0, 4.0 Hz, 1H), 7.72-7.64 (m, 1H), 7.61-7.52 (m, 1H), 4.00 (s, 3H).

¹³C NMR (101 MHz, DMSO-d₆) & 175.27, 145.22, 140.18, 139.62, 133.71, 133.06, 131.04, 129.58, 127.17, 126.12, 123.92, 34.14.

High-resolution mass spectrometry (electrospray ionization)(HRMS (ESI)): m/z theoretical value: C₁₂H₉N₃O₂ [M+H]⁺228.0768, measured value: 228.0771.

1,4-Naphthoquinone imidazole was reacted with halohydrocarbons having different substitutes to prepare Compounds 2-20 according to the method in Example 1; other materials and reaction conditions for the compounds were kept the same and raw materials could be purchased commercially:

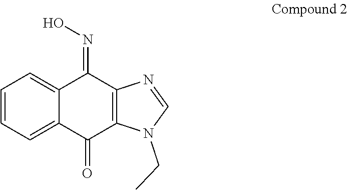 [SMILES: CCn1cnc2c1C(=O)c1ccccc1/C2=N\O]

(E)-3-ethyl-9-(hydroxylimino)-3,9-dihydro-4H-naphthol[2,3-d]imidazole-4-one

¹H NMR (400 MHZ, DMSO-d₆) δ 13.04 (s, 1H), 8.36 (s, 1H), 8.26 (d, J=8.0 Hz, 1H), 8.10 (d, J=8.0 Hz, 1H), 7.72-7.64 (m, 1H), 7.60 (t, J=7.2 Hz, 1H), 4.46 (q, J=7.2 Hz, 2H), 1.39 (t, J=8.0 Hz, 3H).

¹³C NMR (101 MHz, DMSO-d₆) δ 174.94, 144.31, 140.21, 140.08, 133.66, 133.06, 131.10, 129.59, 126.48, 126.22, 123.91, 42.17, 16.64.

HRMS (ESI): m/z theoretical value: C₁₃H₁₁N₃O₂ [M+H]⁺242.0924, measured value: 242.0927.

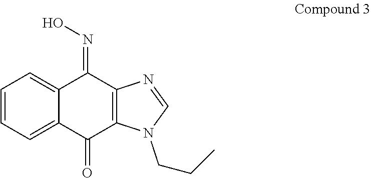 [SMILES: CCCn1cnc2c1C(=O)c1ccccc1/C2=N\O]

(E)-3-propyl-9-(hydroxyimino)-3,9-dihydro-4H-naphtho[2,3-d]imidazol-4-one

¹H NMR (400 MHZ, DMSO-d₆) δ 13.05 (s, 1H), 8.34 (s, 1H), 8.26 (d, J=8.0 Hz, 1H), 8.09 (dd, J=8.0, 1.0 Hz, 1H), 7.72-7.65 (m, 1H), 7.59 (td, J=8.0, 1.0 Hz, 1H), 4.39 (t, J=8.0 Hz, 2H), 1.85-1.72 (m, 2H), 0.83 (t, J=7.4 Hz, 3H).

¹³C NMR (101 MHz, DMSO-d₆) δ 174.99, 144.84, 140.20, 140.05, 133.64, 133.05, 131.11, 129.58, 126.57, 126.21, 123.89, 48.30, 23.97, 11.02.

HRMS (ESI): m/z theoretical value: C₁₄H₁₃N₃O₂ [M+H]⁺256.1081, measured value: 256.1084.

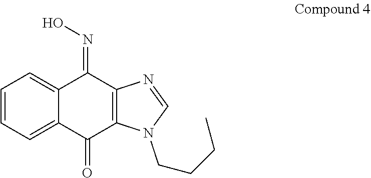 [SMILES: CCCCn1cnc2c1C(=O)c1ccccc1/C2=N\O]

(E)-3-butyl-9-(hydroxyimino)-3,9-dihydro-4H-naphtho[2,3-d]imidazol-4-one

¹H NMR (400 MHZ, DMSO-d₆) δ 13.05 (s, 1H), 8.34 (s, 1H), 8.26 (dd, J=8.0, 1.2 Hz, 1H), 8.09 (dd, J=8.0, 1.2 Hz, 1H), 7.67 (td, J=4.0, 1.6 Hz, 1H), 7.59 (td, J=8.0, 1.2 Hz, 1H), 4.42 (t, J=8.0 Hz, 2H), 1.81-1.66 (m, 2H), 1.30-1.19 (m, 2H), 0.86 (t, J=8.0 Hz, 3H).

¹³C NMR (101 MHz, DMSO-d₆) δ 174.97, 144.77, 140.20, 140.03, 133.63, 133.04, 131.11, 129.57, 126.54, 126.22, 123.89, 46.55, 32.70, 19.48, 13.84.

HRMS (ESI): m/z theoretical value: C₁₅H₁₅N₃O₂ [M+H]⁺270.1237, measured value: 270.1240.

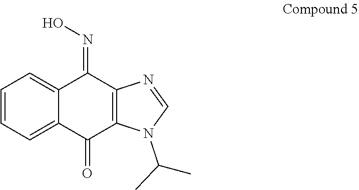 [SMILES: CC(C)n1cnc2c1C(=O)c1ccccc1/C2=N\O]

(E)-3-isopropyl-9-(hydroxylimino)-3,9-dihydro-4H-naphthol[2,3-d]imidazole-4-one

¹H NMR (400 MHZ, DMSO-d₆) δ 13.12 (s, 1H), 8.52 (s, 1H), 8.26 (d, J=8.0 Hz, 1H), 8.11 (dd, J=8.0, 1.6 Hz, 1H), 7.73-7.63 (m, 1H), 7.59 (td, J=8.0, 1.2 Hz, 1H), 5.39-5.25 (m, 1H), 1.51 (d, J=8.0 Hz, 6H).

¹³C NMR (101 MHz, DMSO-d₆) δ 174.95, 141.88, 140.23, 140.16, 133.33, 133.04, 131.27, 129.61, 126.36, 126.16, 123.78, 49.64, 23.06.

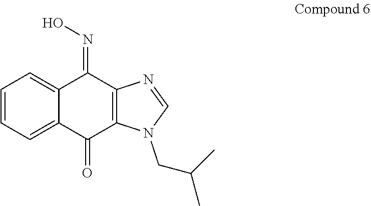 [SMILES: CC(C)Cn1cnc2c1C(=O)c1ccccc1/C2=N\O]

(E)-3-ethyl-isobutyl-9-(hydroxylimino)-3,9-dihydro-4H-naphthol[2,3-d]imidazole-4-one

¹H NMR (400 MHZ, DMSO-d₆) δ 13.05 (s, 1H), 8.33 (s, 1H), 8.27 (dd, J=8.0, 1.2 Hz, 1H), 8.10 (dd, J=8.0, 1.4 Hz, 1H), 7.68 (td, J=8.0, 1.4 Hz, 1H), 7.59 (td, J=8.0, 1.2 Hz, 1H), 4.26 (d, J=8.0 Hz, 2H), 2.17-2.02 (m, 1H), 0.84 (d, J=4.0 Hz, 6H).

¹³C NMR (101 MHz, DMSO-d₆) δ 175.08, 145.17, 140.19, 140.03, 133.64, 133.07, 131.13, 129.59, 126.69, 126.22, 123.90, 53.53, 29.52, 19.75.

HRMS (ESI): m/z theoretical value: C₁₅H₁₅N₃O₂ [M+H]⁺270.1237, measured value: 270.1237.

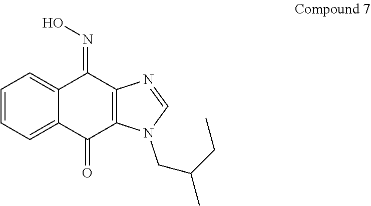 [SMILES: CCC(C)Cn1cnc2c1C(=O)c1ccccc1/C2=N\O]

(E)-9-(hydroxylimino)-3-(2-methylbutyl)-3,9-dihydro-4H-naphthol[2,3-d]

¹H NMR (400 MHZ, DMSO-d₆) δ 13.05 (s, 1H), 8.33 (s, 1H), 8.27 (dd, J=8.0, 1.2 Hz, 1H), 8.10 (dd, J=8.0, 1.4 Hz, 1H), 7.71-7.64 (m, 1H), 7.60 (td, J=8.0, 1.2 Hz, 1H), 4.43-4.35 (m, 1H), 4.24-4.16 (m, 1H), 1.99-1.83 (m, 1H), 1.37-1.20 (m, 1H), 1.17-1.07 (m, 1H), 0.85 (t, J=8.0 Hz, 3H), 0.77 (d, J=4.0 Hz, 3H).

¹³C NMR (101 MHz, DMSO-d₆) δ 175.08, 159.51, 145.25, 140.19, 140.05, 133.65, 133.08, 131.14, 129.60, 126.72, 126.25, 123.90, 52.24, 35.68, 26.51, 16.57, 11.31.

HRMS (ESI): m/z theoretical value: C₁₆H₁₇N₃O₂ [M+H]⁺284.1394, measured value: 284.1390.

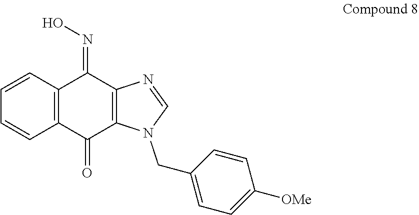 [SMILES: COc1ccc(Cn2cnc3c2C(=O)c2ccccc2/C3=N\O)cc1]

(E)-9-(hydroxylimino)-3-(4-methoxybenzyl)-3,9-dihydro-4H-naphthol[2,3-d]imidazole-4-one

¹H NMR (400 MHZ, DMSO-d₆) δ 13.03 (s, 1H), 8.48 (s, 1H), 8.27 (d, J=8.0 Hz, 1H), 8.09 (dd, J=8.0, 1.2 Hz, 1H), 7.68 (td, J=8.0, 1.2 Hz, 1H), 7.62-7.55 (m, 1H), 7.28 (d, J=8.0 Hz, 2H), 6.86 (d, J=8.0 Hz, 2H), 5.62 (s, 2H).

¹³C NMR (101 MHZ, DMSO-d₆) δ 175.10, 159.52, 159.38, 144.87, 140.11, 133.77, 133.14, 131.03, 129.59, 129.50, 129.35, 126.36, 126.22, 123.96, 114.49, 55.52, 49.10.

HRMS (ESI): m/z theoretical value: C₁₉H₁₅FN₃O₃ [M+H]⁺334.1186, measured value: 334.1182.

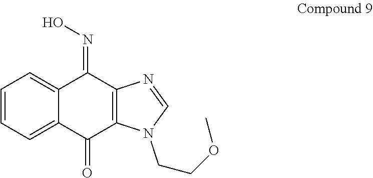 [SMILES: COCCn1cnc2c1C(=O)c1ccccc1/C2=N\O]

(E)-9-(hydroxylimino)-3-(2-methoxyethyl)-3,9-dihydro-4H-naphthol[2,3-d]

¹H NMR (400 MHZ, DMSO-d₆) δ 13.04 (s, 1H), 8.30-8.23 (m, 2H), 8.09 (dd, J=8.0, 1.4 Hz, 1H), 7.68 (td, J=8.0, 1.4 Hz, 1H), 7.59 (td, J=8.0, 1.2 Hz, 1H), 4.61 (t, J=5.2 Hz, 2H), 3.68 (t, J=4.0 Hz, 2H), 3.20 (s, 3H).

¹³C NMR (101 MHz, DMSO-d₆) δ 175.16, 145.37, 140.16, 139.96, 133.69, 133.09, 131.05, 129.57, 126.48, 126.20, 123.93, 70.72, 58.48, 46.46.

HRMS (ESI): m/z theoretical value: C₁₄H₁₃N₃O₃ [M+H]⁺272.1030, measured value: 272.1032.

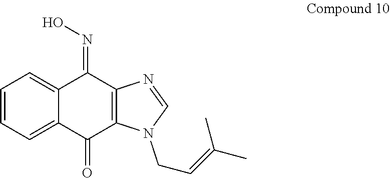 [SMILES: CC(C)=CCn1cnc2c1C(=O)c1ccccc1/C2=N\O]

(E)-9-(hydroxylimino)-3-(3-methyl-2-ene-1-yl)-3,9-dihydro-4H-naphthol[2,3-d]imidazole-4-one

¹H NMR (400 MHZ, DMSO-d₆) δ 13.03 (s, 1H), 8.32 (s, 1H), 8.26 (dd, J=8.0, 1.2 Hz, 1H), 8.10 (dd, J=8.0, 1.4 Hz, 1H), 7.68 (td, J=8.0, 1.4 Hz, 1H), 7.59 (td, J=8.0, 1.2 Hz, 1H), 5.44-5.35 (m, 1H), 5.06 (d, J=8.0 Hz, 2H), 1.77 (s, 3H), 1.67 (s, 3H).

¹³C NMR (101 MHz, DMSO-d₆) δ 175.05, 144.24, 140.17, 139.91, 137.77, 133.66, 133.07, 131.08, 129.58, 126.45, 126.23, 123.92, 119.76, 44.64, 25.77, 18.42.

HRMS (ESI): m/z theoretical value: C₁₆H₁₅N₃O₂ [M+H]⁺282.1237, measured value: 282.1239.

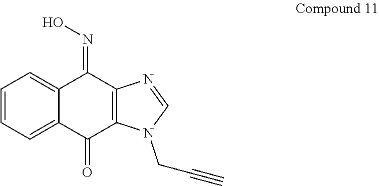 [SMILES: C#CCn1cnc2c1C(=O)c1ccccc1/C2=N\O]

(E)-9-(hydroxylimino)-3-(propyl-2-yne-1-yl)-3,9-dihydro-4H-naphthol[2,3-d]

¹H NMR (400 MHZ, DMSO-d₆) δ 13.02 (s, 1H), 8.41 (s, 1H), 8.28 (d, J=8.0 Hz, 1H), 8.11 (d, J=8.0 Hz, 1H), 7.73-7.64 (m, 1H), 7.64-7.54 (m, 1H), 5.35 (s, 2H), 3.53 (s, 1H).

¹³C NMR (101 MHz, DMSO-d₆) δ 175.02, 144.29, 139.99, 133.91, 133.22, 130.86, 129.61, 129.58, 126.61, 126.12, 124.05, 78.38, 77.22, 36.50.

HRMS (ESI): m/z theoretical value: C₁₄H₉N₃O₂ [M+H]⁺252.0768, measured value: 252.0769.

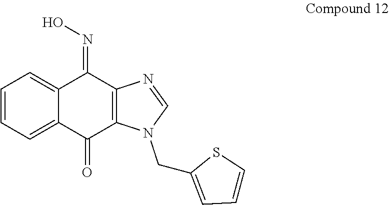 [SMILES: O=C1c2ccccc2/C(=N\O)c2ncn(Cc3cccs3)c21]

(E)-9-(hydroxylimino)-3-(thiophene-2-yl-methyl)-3,9-dihydro-4H-naphthol[2,3-d]imidazole-4-one

¹H NMR (400 MHZ, DMSO-d₆) δ 13.01 (s, 1H), 8.49 (s, 1H), 8.27 (d, J=8.0, 1H), 8.12 (dd, J=7.9, 1.5 Hz, 1H), 7.69 (t, J=8.0, 1H), 7.60 (t, J=8.0 Hz, 1H), 7.43 (d, J=8.0 Hz, 1H), 7.19 (d, J=4.0 Hz, 1H), 6.95 (dd, J=8.0, 4.0 Hz, 1H), 5.88 (s, 2H).

¹³C NMR (101 MHZ, DMSO-d₆) δ 175.20, 144.72, 140.04, 139.91, 139.42, 133.87, 133.20, 130.93, 129.60, 127.89, 127.49, 127.25, 126.19,126.18, 124.02, 44.36.

HRMS (ESI): m/z theoretical value: C₁₆H₁₁N₃O₂S [M+H]⁺310.0645, measured value: 310.0642.

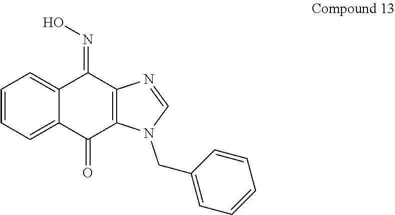 [SMILES: O=C1c2ccccc2/C(=N\O)c2ncn(Cc3ccccc3)c21]

(E)-9-(hydroxylimino)-3-benzyl-3,9-dihydro-4H-naphthol[2,3-d]imidazole-4-one

¹H NMR (400 MHZ, DMSO-d₆) δ 13.05 (s, 1H), 8.51 (s, 1H), 8.27 (dd, J=8.0, 1.2 Hz, 1H), 8.07 (dd, J=8.0, 1.4 Hz, 1H), 7.67 (td, J=8.0 1.4 Hz, 1H), 7.57 (td, J=8.0, 1.2 Hz, 1H), 7.39-7.10 (m, 5H), 5.70 (s, 2H).

¹³C NMR (101 MHZ, DMSO-d₆) δ 175.08, 145.10, 140.10, 140.06, 137.44, 133.81, 133.14, 130.98, 129.56, 129.12, 128.25, 127.72, 126.48, 126.18, 123.97, 49.58.

HRMS (ESI): m/z theoretical value: C₁₈H₁₃N₃O₂ [M+H]⁺304.1081, measured value: 304.1082.

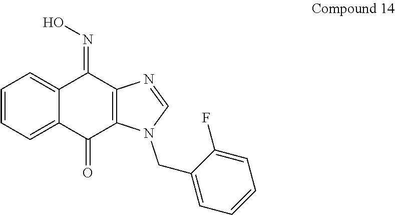 [SMILES: O=C1c2ccccc2/C(=N\O)c2ncn(Cc3ccccc3F)c21]

(E)-9-(hydroxylimino)-3-(2-fluorobenzyl)-3,9-dihydro-4H-naphthol[2,3-d]imidazole-4-one

¹H NMR (400 MHZ, DMSO-d₆) δ 13.04 (s, 1H), 8.41 (s, 1H), 8.31-8.22 (m, 1H), 8.04 (dd, J=8.0, 1.4 Hz, 1H), 7.70-7.64 (m, 1H), 7.62-7.54 (m, 1H), 7.35-7.27 (m, 1H), 7.25-7.18 (m, 1H), 7.10 (td, J=7.5, 1.2 Hz, 1H), 7.04 (td, J=8.0, 1.8 Hz, 1H), 5.77 (s, 2H).

¹³C NMR (101 MHz, DMSO-d₆) δ 175.00, 163.05 (160.71, J=234.0 Hz), 145.45, 140.08, 139.99, 133.88, 133.16, 130.90, 130.45 (130.37, J=8.0 Hz), 129.56, 129.35 (129.31, J=4.0 Hz), 126.66, 126.15, 125.19 (125.15, J=4.0 Hz), 124.45 (124.31, J=14.0 Hz), 123.99, 115.92 (115.71, J=21.0 Hz), 44.17 (44.12, J=5.0 Hz).

HRMS (ESI): m/z theoretical value: C₁₈H₁₂FN₃O₂ [M+H]⁺322.0986, measured value: 322.0984.

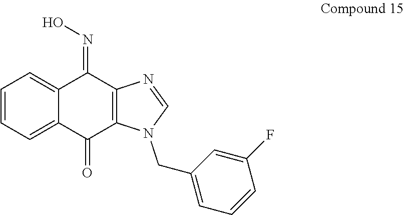 [SMILES: O=C1c2ccccc2/C(=N\O)c2ncn(Cc3cccc(F)c3)c21]

(E)-9-(hydroxylimino)-3-(3-fluorobenzyl)-3,9-dihydro-4H-naphthol[2,3-d]

¹H NMR (400 MHZ, DMSO-d₆) δ 8.51 (s, 1H), 8.27 (d, J=8.0 Hz, 1H), 8.07 (d, J=8.0 Hz, 1H), 7.68 (t, J=8.0 Hz, 1H), 7.58 (t, J=8.0 Hz, 1H), 7.40-7.30 (m, 1H), 7.17-7.06 (m, 3H), 5.71 (s, 2H).

¹³C NMR (101 MHz, DMSO-d₆) δ 175.12, 163.82 (161.39, J=243.0 Hz), 145.17, 140.22 (140.15, J=7.0 Hz), 140.10, 133.87, 133.17, 131.22 (131.14, J=8.0 Hz), 130.95, 129.56, 126.63, 126.47, 126.18, 123.99, 123.75 (123.72, J=3.0 Hz), 115.21 (115.00, J=21.0 Hz), 114.78 (114.56, J=22.0 Hz), 49.06 (49.05, J=1.0 Hz).

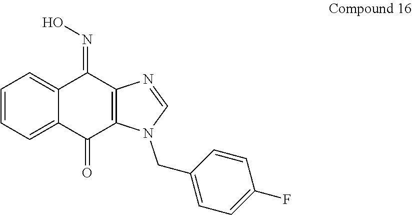 [SMILES: O=C1c2ccccc2/C(=N\O)c2ncn(Cc3ccc(F)cc3)c21]

(E)-9-(hydroxylimino)-3-(4-fluorobenzyl)-3,9-dihydro-4H-naphthol[2,3-d]imidazole-4-one

¹H NMR (400 MHZ, DMSO-d₆) δ 13.04 (s, 1H), 8.51 (s, 1H), 8.27 (dd, J=8.0, 1.2 Hz, 1H), 8.07 (dd, J=8.0, 1.4 Hz, 1H), 7.68 (td, J=8.0, 1.4 Hz, 1H), 7.58 (td, J=8.0, 1.2 Hz, 1H), 7.39-7.31 (m, 2H), 7.18-7.07 (m, 2H), 5.68 (s, 2H).

¹³C NMR (101 MHZ, DMSO-d₆) δ 175.11, 163.34 (160.91, J=243.0 Hz), 145.01, 140.13, 140.09, 133.83, 133.65 (133.62, J=3.0 Hz), 133.15, 130.96, 130.15 (130.07, J=8.0 Hz), 129.56, 126.40, 126.19, 123.97, 116.03 (115.81, J=22.0 Hz), 48.87.

HRMS (ESI): m/z theoretical value: C₁₈H₁₂FN₃O₂ [M+H]⁺322.0986, measured value: 322.0986.

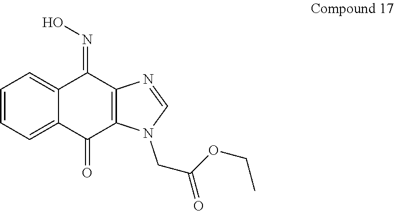 [SMILES: CCOC(=O)Cn1cnc2c1C(=O)c1ccccc1/C2=N\O]

(E)-2-(4-(hydroxylimino)-9-oxo-4,9-dihydro-1H-naphthol[2,3-d]imidazole-1-yl)acetate

¹H NMR (400 MHZ, DMSO-d₆) δ 13.01 (s, 1H), 8.31-8.25 (m, 2H), 8.06 (dd, J=8.0, 0.8 Hz, 1H), 7.70 (td, J=8.0, 1.2 Hz, 1H), 7.60 (td, J=8.0, 0.8 Hz, 1H), 5.32 (s, 2H), 4.15 (q, J=7.2 Hz, 2H), 1.19 (t, J=7.2 Hz, 3H).

¹³C NMR (101 MHz, DMSO-d₆) δ 175.27, 168.10, 145.42, 134.05, 133.27, 130.71, 129.61, 127.16, 126.61, 126.05, 124.13, 61.78, 48.16, 14.47.

HRMS (ESI): m/z theoretical value: C₁₅H₁₃N₃O₄ [M+H]⁺300.0979, measured value: 300.0981.

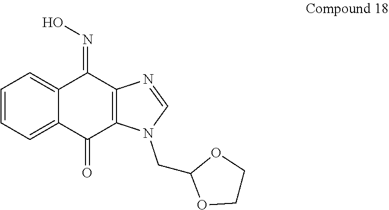 [SMILES: O=C1c2ccccc2/C(=N\O)c2ncn(CC3OCCO3)c21]

(E)-3-((1,3-ethyl-dioxoheterocycle-2-yl)methyl)-9-(hydroxylimino)-3,9-dihydro-4H-naphthol[2,3-d]imidazole-4-one

¹H NMR (400 MHZ, DMSO-d₆) δ 13.03 (s, 1H), 8.30-8.20 (m, 2H), 8.09 (dd, J=8.0, 1.4 Hz, 1H), 7.68 (td, J=8.0, 1.4 Hz, 1H), 7.59 (td, J=8.0, 1.2 Hz, 1H), 5.20 (t, J=4.0 Hz, 1H), 4.67 (d, J=4.0 Hz, 2H), 3.82-3.73 (m, 4H).

¹³C NMR (101 MHz, DMSO-d₆) δ 175.26, 145.49, 140.06, 139.55, 133.72, 133.12, 131.03, 129.58, 126.88, 126.19, 123.95, 101.10, 65.05, 48.12.

HRMS (ESI): m/z theoretical value: C₁₅H₁₃N₃O₄ [M+H]⁺300.0979, measured value: 300.0978.

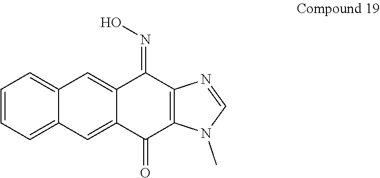 [SMILES: Cn1cnc2c1C(=O)c1cc3ccccc3cc1/C2=N\O]

(E)-11-(hydroxylimino)-3-methyl-3,11-dihydro-4H-anthraquinone[2,3-d]imidazole-4-one

¹H NMR (400 MHZ, DMSO-d₆) δ 13.09 (s, 1H), 9.04 (s, 1H), 8.77 (d, J=12.4 Hz, 2H), 8.15 (dd, J=26.4, 7.7 Hz, 2H), 7.64 (d, J=20.0 Hz, 2H), 4.03 (s, 3H).

HRMS (ESI): m/z theoretical value: C₁₅H₁₃N₃O₄ [M+H]⁺278.0924, measured value: 278.0922.

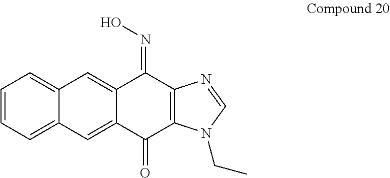 [SMILES: CCn1cnc2c1C(=O)c1cc3ccccc3cc1/C2=N\O]

(E)-11-(hydroxylimino)-3-ethyl-3,11-dihydro-4H-anthraquinone[2,3-d]imidazole-4-one

¹H NMR (400 MHZ, DMSO-d₆) δ 13.89 (s, 1H), 8.93 (s, 1H), 8.84 (s, 2H), 8.21 (dd, J=22.4, 8.2 Hz, 2H), 7.82-7.64 (m, 2H), 4.60 (d, J=7.8 Hz, 2H), 1.41 (t, J=8.0 Hz, 3H).

HRMS (ESI): m/z theoretical value: C₁₅H₁₃N₃O₄ [M+H]⁺292.1081, measured value: 292.1078.

### Example 2

2,2′-(1,4-dioxy-1,4,6,7-tetrahydronaphthalene-2,3-diyl)bis(isoindolin-1,3-dione) (13.3 g) was dissolved into 200 mL of dichloromethane and 20 mL of methanol with the addition of cyclopropylamine, and subjected to reaction for 2 h, then solvent was removed, the obtained product was added with 100 mL of water, pulped for 10 min, and subjected to suction filtration to obtain red solid (10 g). 10 g solid was directly added to 150 mL of ethanol; 8 mL hydrazine hydrate (80) was dropwisely added, and the mixture was stirred at room temperature for half an hour; organic phase was removed; 200 mL of water was added, and the obtained product was pulped for 10 min, filtered and washed with water until colorless, and then vacuum dried to obtain blue solid 2-amino-3-cyclopropyl-6,7-dihydronaphthalene-1,4-dione (6.2 g). 2-amino-3-cyclopropyl-6,7-dihydronaphthalene-1,4-dione (1 g) was subjected to reflux for 2.5 h in an orthoformic acid medium (25 mL), and solvent was removed, and the remaining material was isolated by silica gel column chromatography to obtain white solid 1-cyclopropyl 6,7-dihydro-1H-naphthol[2,3-d]imidazole-4,9-dione (0.46 g). 3-Cyclopropylnaphthoquinone imidazole (0.21 g), hydroxylamine hydrochloride (0.35 g), and pyridine (0.8 mL) were dissolved into ethanol (70 mL) and reacted for 36 h at 100° C.; at the end of reaction, solid was separated out from solution, directly subjected to suction filtration, and vacuum dried to obtain light yellow solid (0.15 g).

Compound 21 could be prepared using the method in Example 2:

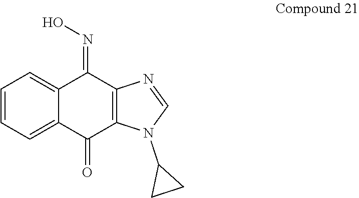 [SMILES: O=C1c2ccccc2/C(=N\O)c2ncn(C3CC3)c21]

(E)-3-cyclopropyl-9-(hydroxylimino)-3,9-dihydro-4H-naphthol[2,3-d]imidazole-4-one

¹H NMR (400 MHZ, DMSO-d₆) δ 13.00 (s, 1H), 8.31 (s, 1H), 8.25 (dd, J=8.0, 1.2 Hz, 1H), 8.11 (dd, J=8.0, 1.4 Hz, 1H), 7.67 (td, J=8.0, 1.4 Hz, 1H), 7.59 (td, J=8.0, 1.2 Hz, 1H), 3.93-3.86 (m, 1H), 1.13-1.03 (m, 4H).

¹³C NMR (101 MHZ, DMSO-d₆) δ 174.50, 143.32, 140.18, 139.97, 133.46, 132.97, 131.25, 129.58, 128.36, 126.23, 123.86, 29.61, 7.44.

HRMS (ESI): m/z theoretical value: C₁₄H₁₁N₃O₂ [M+H]⁺254.0924, measured value: 254.0927.

### Example 3

Nitrogen-(3-chloro-1,4-dioxy-1,4,6,7-tetrahydronaphthalene-2-yl) acetamide (0.25 g) was dissolved into 20 mL of tetrahydrofuran; reaction liquid was added with triethylamine and tert-butylamine in turn, subjected to reflux reaction for 26 h under nitrogen protection, and cooled to room temperature, and then solvent was removed, the remaining solution was added with 10 mL of ultrapure water, and pulped for half an hour to obtain nitrogen-(3-(tert-butylamino)-1,4-dioxy-1,4,6,7-tetrahydronaphthalene-2-yl) acetamide. The obtained product was dispersed into 85 mL of hydrazine hydrate (50%) for reaction for 2 h at 80° C. and extracted with dichloromethane; organic phase was collected, washed with saturated salt solution, dried with anhydrous sodium sulfate, filtered, concentrated, and isolated by silica gel column chromatography to obtain a product 2-amino-3-(tert-butylamino)-6,7-dihydronaphthalene-1,4-dione. 2-Amino-3-(tert-butylamino)-6,7-dihydronaphthalene-1,4-dione (1.63 g) was added with 50 mL of triethyl orthoformate for reflux reaction for 3 h, and then cooled to room temperature; solvent was removed, isolated and purified by silica gel column chromatography to obtain a product 3-tert-butylnaphthoquinone imidazole. 3-tert-butylnaphthoquinone imidazole (0.21 g), hydroxylamine hydrochloride (0.17 g), and pyridine (0.4 mL) were dissolved into ethanol (50 mL) and reacted for 24 h at 110° C.; at the end of reaction, solid was separated out from solution, directly subjected to suction filtration, and vacuum dried to obtain light yellow solid (0.16 g).

Amines having different substitutes were added to prepare Compounds 22-32 according to the method in Example 3; other materials and reaction conditions for the compounds were kept the same and raw materials could be purchased commercially:

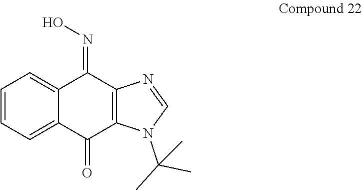 [SMILES: CC(C)(C)n1cnc2c1C(=O)c1ccccc1/C2=N\O]

(E)-3-tert-butyl-9-(hydroxylimino)-3,9-dihydro-4H-naphthol[2,3-d]imidazole-4-one

¹H NMR (400 MHZ, DMSO-d₆) δ 13.55 (s, 1H), 8.49 (s, 1H), 8.23 (d, J=8.0 Hz, 1H), 8.17 (d, J=7.4 Hz, 1H), 7.68 (t, J=8.0 Hz, 1H), 7.60 (t, J=8.0 Hz, 1H), 1.72 (s, 9H).

¹³C NMR (101 MHZ, DMSO-d₆) δ 173.70, 142.87, 142.71, 140.17, 132.99, 132.43, 131.66, 129.80, 127.11, 127.08, 123.26, 59.59, 29.32.

HRMS (ESI): m/z theoretical value: C₁₅H₁₅N₃O₂ [M+H]⁺270.1237, measured value: 270.1241.

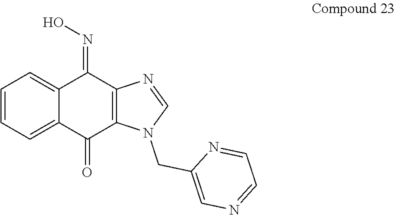 [SMILES: O=C1c2ccccc2/C(=N\O)c2ncn(Cc3cnccn3)c21]

(E)-9-(hydroxylimino)-3-(pyrazine-2-yl-methyl)-3,9-dihydro-4H-naphthol[2,3-d]

¹H NMR (400 MHZ, DMSO-d₆) δ 13.06 (s, 1H), 8.71 (s, 1H), 8.59-8.41 (m, 3H), 8.27 (d, J=8.0 Hz, 1H), 7.99 (d, J=8.0 Hz, 1H), 7.72-7.51 (m, 2H), 5.87 (s, 2H).

¹³C NMR (101 MHz, DMSO-d₆) δ 174.99, 151.72, 145.78, 144.60, 144.34, 143.98, 140.12, 139.84, 133.89, 133.16, 130.80, 129.55, 126.79, 126.08, 124.01, 49.11.

HRMS (ESI): m/z theoretical value: C₁₆H₁₁N₅O₂ [M+H]⁺306.0986, measured value: 306.0985.

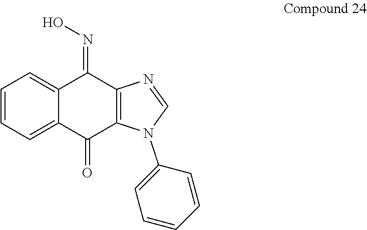 [SMILES: O=C1c2ccccc2/C(=N\O)c2ncn(-c3ccccc3)c21]

(E)-9-(hydroxylimino)-3-benzyl-3,9-dihydro-4H-naphthol[2,3-d]imidazole-4-one

¹H NMR (400 MHZ, DMSO-d₆) δ 13.10 (s, 1H), 8.47 (s, 1H), 8.30 (dd, J=8.0, 1.2 Hz, 1H), 8.02 (dd, J=8.0, 1.4 Hz, 1H), 7.69 (td, J=8.0, 1.4 Hz, 1H), 7.63-7.48 (m, 6H).

¹³C NMR (101 MHz, DMSO-d₆) δ 174.10, 145.03, 140.19, 140.10, 135.90, 133.66, 133.07, 131.24, 129.59, 129.36, 129.31, 127.14, 126.57, 126.27, 123.91.

HRMS (ESI): m/z theoretical value: C₁₇H₁₁N₃O₂ [M+H]⁺290.0924, measured value: 290.0922.

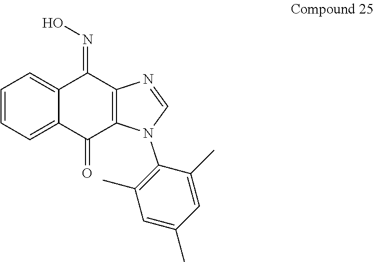 [SMILES: Cc1cc(C)c(-n2cnc3c2C(=O)c2ccccc2/C3=N\O)c(C)c1]

(E)-9-(hydroxylimino)-3-(2,4,6-trimethylphenyl)-3,9-dihydro-4H-naphthol[2,3-d]imidazole-4-one

¹H NMR (400 MHZ, DMSO-d₆) δ 13.09 (s, 1H), 8.35-8.26 (m, 2H), 8.00-7.95 (m, 1H), 7.73-7.65 (m, 1H), 7.62-7.54 (m, 1H), 7.03 (s, 2H), 3.34 (s, 9H).

¹³C NMR (101 MHz, DMSO-d₆) δ 174.25, 144.93, 140.19, 139.83, 138.97, 135.02, 133.96, 133.14, 132.50, 130.95, 129.58, 129.12, 127.34, 126.10, 124.05, 21.06, 17.48.

HRMS (ESI): m/z theoretical value: C₂₀H₁₇N₃O₂ [M+H]⁺322.1394, measured value: 322.1392.

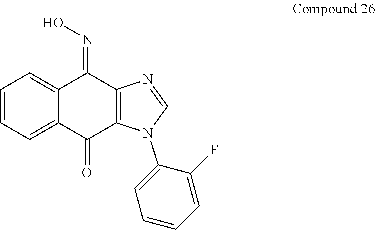 [SMILES: O=C1c2ccccc2/C(=N\O)c2ncn(-c3ccccc3F)c21]

(E)-9-(hydroxylimino)-3-(2-fluorophenyl)-3,9-dihydro-4H-naphthol[2,3-d]imidazole-4-one

¹H NMR (400 MHZ, DMSO-d₆) δ 13.09 (s, 1H), 8.50 (s, 1H), 8.32 (dd, J=8.0, 1.2 Hz, 1H), 8.01 (dd, J=8.0, 1.4 Hz, 1H), 7.75-7.65 (m, 2H), 7.64-7.56 (m, 2H), 7.51-7.44 (m, 1H), 7.38 (td, J=8.0, 1.2 Hz, 1H).

¹³C NMR (101 MHZ, DMSO-d₆) δ 174.23, 158.18 (155.70, J=248.0 Hz), 145.23, 139.94 (139.68, J=26.0 Hz), 133.93, 133.26, 131.86 (131.78, J=8.0 Hz), 130.84, 129.64, 129.18, 127.94, 127.80, 126.10, 125.33 (125.29, J=4.0 Hz), 124.21, 124.10, 116.75 (116.55, J=20.0 Hz).

HRMS (ESI): m/z theoretical value: C₁₇H₁₀FN₃O₂ [M+H]⁺308.0830, measured value: 308.0831.

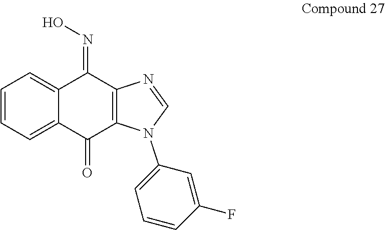 [SMILES: O=C1c2ccccc2/C(=N\O)c2ncn(-c3cccc(F)c3)c21]

(E)-9-(hydroxylimino)-3-(3-fluorophenyl)-3,9-dihydro-4H-naphthol[2,3-d]imidazole-4-one

¹H NMR (400 MHZ, DMSO-d₆) δ 13.08 (s, 1H), 8.49 (s, 1H), 8.30 (dd, J=8.0, 1.2 Hz, 1H), 8.03 (dd, J=8.0, 1.4 Hz, 1H), 7.70 (td, J=8.0, 1.4 Hz, 1H), 7.62-7.54 (m, 3H), 7.48-7.43 (m, 1H), 7.42-7.35 (m, 1H).

¹³C NMR (101 MHz, DMSO-d₆) δ 174.15, 163.26 (160.83, J=243.0 Hz), 145.03, 140.17 (140.02, J=15.0 Hz), 137.30, 137.19, 133.70, 133.11, 131.17, 130.96 (130.87, J=9.0 Hz), 129.58, 127.19, 126.28, 123.92, 122.87 (122.84, J=3.0 Hz), 116.42 (116.21, J=21.0 Hz), 114.56 (114.31, J=25.0 Hz).

HRMS (ESI): m/z theoretical value: C₁₇H₁₀FN₃O₂ [M+H]⁺308.0830, measured value: 308.0832.

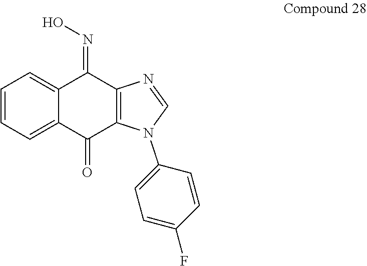 [SMILES: O=C1c2ccccc2/C(=N\O)c2ncn(-c3ccc(F)cc3)c21]

(E)-9-(hydroxylimino)-3-(4-fluorophenyl)-3,9-dihydro-4H-naphthol[2,3-d]imidazole-4-one

¹H NMR (400 MHz, DMSO-d₆) δ 13.07 (s, 1H), 8.45 (s, 1H), 8.32-8.27 (m, 1H), 8.02 (dd, J=8.0, 1.4 Hz, 1H), 7.72-7.62 (m, 3H), 7.61-7.55 (m, 1H), 7.41-7.35 (m, 2H).

¹³C NMR (101 MHz, DMSO-d₆) δ 174.18, 163.67 (161.22, J=245.0 Hz), 145.09, 140.08, 133.72, 133.10, 132.30 (132.27, J=3.0 Hz), 131.19, 129.59, 128.95 (128.86, J=9.0 Hz), 127.28, 126.60, 126.24, 123.93, 116.22 (115.99, J=23.0 Hz).

HRMS (ESI): m/z theoretical value: C₁₇H₁₀FN₃O₂ [M+H]⁺308.0830, measured value: 308.0829.

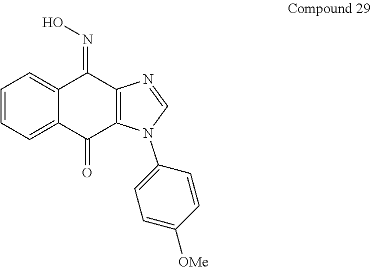 [SMILES: COc1ccc(-n2cnc3c2C(=O)c2ccccc2/C3=N\O)cc1]

(E)-9-(hydroxylimino)-3-(4-methoxyphenyl)-3,9-dihydro-4H-naphthol[2,3-d]imidazole-4-one

¹H NMR (400 MHZ, DMSO-d₆) δ 13.07 (s, 1H), 8.40 (s, 1H), 8.32-8.28 (m, 1H), 8.02 (dd, J=8.0, 1.4 Hz, 1H), 7.70 (td, J=8.0, 1.4 Hz, 1H), 7.59 (td, J=8.0, 1.2 Hz, 1H), 7.51-7.47 (m, 2H), 7.08-7.04 (m, 2H), 3.82 (s, 3H).

¹³C NMR (101 MHZ, DMSO-d₆) δ 174.10, 159.95, 159.53, 145.06, 140.16, 133.63, 133.05, 131.28, 129.59, 128.73, 127.82, 127.23, 126.25, 123.90, 114.40, 56.00.

HRMS (ESI): m/z theoretical value: C₁₈H₁₃FN₃O₃ [M+H]⁺320.1030, measured value: 320.1026.

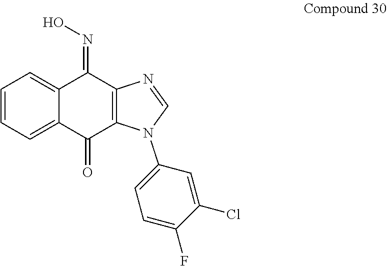 [SMILES: O=C1c2ccccc2/C(=N\O)c2ncn(-c3ccc(F)c(Cl)c3)c21]

(E)-9-(hydroxylimino)-3-(3-chloro-4-fluorophenyl)-3,9-dihydro-4H-naphthol[2,3-d]imidazole-4-one

¹H NMR (400 MHZ, DMSO-d₆) δ 13.13 (s, 1H), 8.52 (s, 1H), 8.30 (dd, J=8.0, 1.2 Hz, 1H), 8.05-7.97 (m, 2H), 7.72-7.64 (m, 2H), 7.62-7.55 (m, 2H).

¹³C NMR (101 MHz, DMSO-d₆) δ 174.25, 158.99 (156.52, J=247.0 Hz), 145.02, 139.91, 133.71, 133.19, 132.90 (132.87, J=3.0 Hz), 131.06, 129.61, 129.20, 127.83, 127.75, 127.28, 126.26, 123.95, 120.02 (119.83, J=19.0 Hz), 117.46 (117.24 J=22.0 Hz).

HRMS (ESI): m/z theoretical value: C₁₇H₉ClFN₃O₂ [M+H]⁺342.0440, measured value: 342.0438.

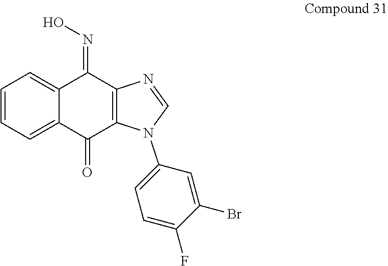 [SMILES: O=C1c2ccccc2/C(=N\O)c2ncn(-c3ccc(F)c(Br)c3)c21]

(E)-9-(hydroxylimino)-3-(3-bromo-4-fluorophenyl)-3,9-dihydro-4H-naphthol[2,3-d]imidazole-4-one

¹H NMR (400 MHZ, DMSO-d₆) δ 13.06 (s, 1H), 8.47 (s, 1H), 8.30 (dd, J=8.0, 1.2 Hz, 1H), 8.10-8.00 (m, 2H), 7.72-7.66 (m, 2H), 7.61-7.53 (m, 2H).

¹³C NMR (101 MHz, DMSO-d₆) δ 174.27, 159.55, 145.12, 140.03 (139.98, J=5.0 Hz), 133.77, 133.16, 133.15, 131.83, 131.11, 129.59, 128.45, 128.37, 127.34, 126.25, 123.96, 117.20 (116.96, J=24.0 Hz), 108.33 (108.11, J=22.0 Hz).

HRMS (ESI): m/z theoretical value: C₁₇H₉BrFN₃O₂ [M+H]⁺385.9935, measured value: 385.9930.

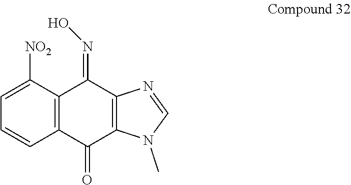 [SMILES: Cn1cnc2c1C(=O)c1cccc([N+](=O)[O-])c1/C2=N\O]

(E)-9-(hydroxylimino)-3-methyl-8-nitro-3,9-dihydro-4H-naphthol[2,3-d]imidazole-4-one

¹H NMR (400 MHZ, DMSO-d₆) δ 14.67 (s, 1H), 9.66 (s, 1H), 8.37 (d, J=7.8 Hz, 1H), 8.25 (d, J=7.9 Hz, 1H), 8.09 (t, J=7.8 Hz, 1H), 4.14 (s, 3H).

HRMS (ESI): m/z theoretical value: C₁₇H₉BrFN₃O₂ [M+H]⁺273.0619, measured value: 273.0620.

### Example 4

2,3-Diamine-1,4-benzoquinone (20 g) was dissolved into 100 mL of acetic acid, subjected to reflux reaction for 6 h, and cooled to room temperature, then poured into an ice-water mixture with the addition of ammonium hydroxide to regulate pH to 9-10, and the obtained solution was subjected to suction filtration and vacuum dried to obtain yellow solid 1,4-naphthoquinone imidazole (22 g). 1,4-naphthoquinone imidazole (0.3 g) and potassium hydroxide (0.15 g) were added to dimethyl sulfoxide solution (3 mL); the mixture was stirred at 50° C. for 30 min and added with iodomethane (0.2 mL), after reaction for 2, water was added to separate out solid; the solid was subjected to suction filtration, washed with water, and vacuum dried to obtain yellow solid 2-methyl-1,4-naphthoquinone imidazole (0.31 g). 2-methylnaphthoquinone imidazole (0.2 g), hydroxylamine hydrochloride (0.28 g), and pyridine (0.7 mL) were dissolved into ethanol (70 mL) and reacted for 36 h at 105° C.; at the end of reaction, solid was separated out from solution, directly subjected to suction filtration, and vacuum dried to obtain light yellow solid (0.16 g).

2,3, -Diamine-1,4-naphthoquinone (20 g) was dissolved into 100 mL of acetic acid and subjected to reflux reaction for 6 h to obtain anthraquinone imidazole; or halohydrocarbons having different substitutes were added to prepare Compounds 33-41 according to the method in Example 4; other materials and reaction conditions for the compounds were kept the same and raw materials could be purchased commercially:

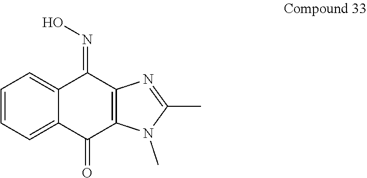 [SMILES: Cc1nc2c(n1C)C(=O)c1ccccc1/C2=N\O]

(E)-9-(hydroxylimino)-2,3-dimethyl-3,9-dihydro-4H-naphthol[2,3-d]imidazole-4-one

¹H NMR (400 MHZ, DMSO-d₆) δ 13.01 (s, 1H), 8.26 (d, J=0.8 Hz, 2H), 8.07 (dd, J=8.0, 4.0 Hz, 1H), 7.72-7.64 (m, 1H), 7.61-7.52 (m, 1H), 4.00 (s, 3H), 2.81 (s, 3H).

¹³C NMR (101 MHZ, DMSO-d₆) δ 175.27, 145.22, 140.18, 139.62, 133.71, 133.06, 131.04, 129.58, 127.17, 126.12, 123.92, 34.14, 20.1.

HRMS (ESI): m/z theoretical value: C₁₂H₉N₃O₂ [M+H]⁺241.0851, measured value: 241.0854.

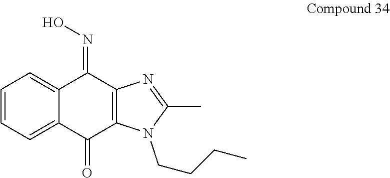 [SMILES: CCCCn1c(C)nc2c1C(=O)c1ccccc1/C2=N\O]

(E)-3-butyl-9-(hydroxylimino)-2-methyl-3,9-dihydro-4H-naphthol[2,3-d]imidazole-4-one

¹H NMR (400 MHZ, DMSO-d₆) δ 13.14 (s, 1H), 8.27 (d, J=7.9 Hz, 1H), 8.12 (dd, J=7.8, 1.5 Hz, 1H), 7.73-7.65 (m, 1H), 7.65-7.57 (m, 1H), 4.41 (d, J=15.1 Hz, 2H), 1.73-1.65 (m, 2H), 1.32 (q, J=7.4 Hz, 2H), 0.90 (t, J=7.3 Hz, 3H).

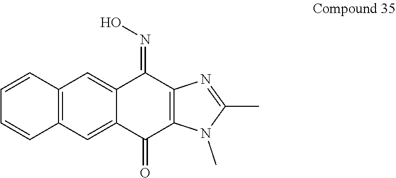 [SMILES: Cc1nc2c(n1C)C(=O)c1cc3ccccc3cc1/C2=N\O]

(E)-11-(hydroxylimino)-2,3-dimethyl-3,11-dihydro-4H-anthraquinone[2,3-d]imidazole-4-one

¹H NMR (400 MHZ, DMSO-d₆) δ 13.09 (s, 1H), 8.77 (d, J=12.4 Hz, 2H), 8.15 (dd, J=26.4, 7.8 Hz, 2H), 7.64 (d, J=20.0 Hz, 2H), 4.03 (s, 3H), 2.54 (s, 3H).

HRMS (ESI): m/z theoretical value: C₁₂H₉N₃O₂ [M+H]⁺292.1081, measured value: 292.1082.

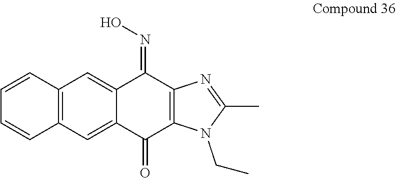 [SMILES: CCn1c(C)nc2c1C(=O)c1cc3ccccc3cc1/C2=N\O]

(E)-3-ethyl-11-(hydroxylimino)-2-methyl-3,11-dihydro-4H-anthraquinone[2,3-d]imidazole-4-one

¹H NMR (400 MHZ, DMSO-d₆) δ 13.87 (s, 1H), 8.84 (s, 2H), 8.21 (dd, J=22.4, 8.2 Hz, 2H), 7.82-7.64 (m, 2H), 4.60 (d, J=7.8 Hz, 2H), 2.79 (s, 3H), 1.40 (d, J=8.0 Hz, 3H).

HRMS (ESI): m/z theoretical value: C₁₂H₉N₃O₂ [M+H]⁺306.1237, measured value: 306.1240.

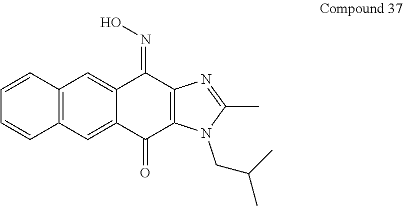 [SMILES: Cc1nc2c(n1CC(C)C)C(=O)c1cc3ccccc3cc1/C2=N\O]

(E)-3-isobutyl-11-(hydroxylimino)-2-methyl-3,11-dihydro-4H-anthraquinone[2,3-d]imidazole-4-one

¹H NMR (400 MHZ, DMSO-d₆) δ 13.90 (s, 1H), 8.84 (s, 2H), 8.20 (dd, J=15.0, 8.4 Hz, 2H), 7.81-7.58 (m, 2H), 4.41 (d, J=7.0 Hz, 2H), 2.78 (s, 3H), 2.22-2.14 (m, 1H), 0.95 (d, J=6.8 Hz, 6H).

HRMS (ESI): m/z theoretical value: C₁₂H₉N₃O₂ [M+H]⁺344.1550, measured value: 344.1554.

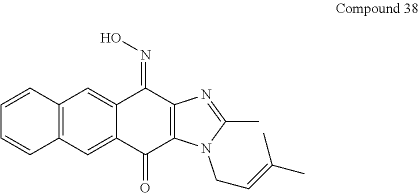 [SMILES: CC(C)=CCn1c(C)nc2c1C(=O)c1cc3ccccc3cc1/C2=N\O]

(E)-11-(hydroxylimino)-2-methyl-3-(3-methyl-2-ene-1-yl)-3,11-dihydro-4H-anthraquinone [2,3-d]imidazole-4-one

¹H NMR (400 MHZ, DMSO-d₆) δ 13.44 (s, 1H), 8.81 (s, 2H), 8.24-8.13 (m, 2H), 7.74-7.66 (m, 2H), 5.30-5.22 (m, 2H), 5.14-5.10 (m, 1H), 2.65 (s, 3H), 1.84 (s, 3H), 1.70 (s, 3H).

HRMS (ESI): m/z theoretical value: C₁₂H₉N₃O₂ [M+H]⁺346.1550, measured value: 346.1555.

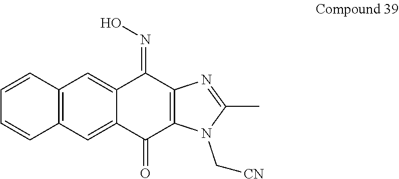 [SMILES: Cc1nc2c(n1CC#N)C(=O)c1cc3ccccc3cc1/C2=N\O]

(E)-2-(4-(hydroxylimino)-2-methyl-11-oxo-4,11-dihydro-1H-anthracene[2,3-d]imidazole-1-yl) acetonitrile

¹H NMR (400 MHZ, DMSO-d₆) δ 13.46 (s, 1H), 8.69 (s, 2H), 8.26 (s, 2H), 7.76 (d, J=6.4 Hz, 2H), 5.70 (s, 2H), 2.59 (s, 3H).

HRMS (ESI): m/z theoretical value: C₁₂H₉N₃O₂ [M+H]⁺317.1033, measured value: 317.1035.

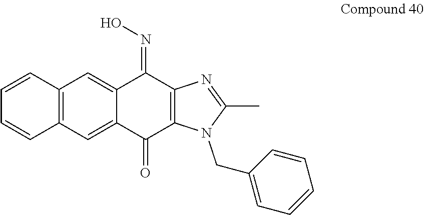 [SMILES: Cc1nc2c(n1Cc1ccccc1)C(=O)c1cc3ccccc3cc1/C2=N\O]

(E)-3-benzyl-11-(hydroxylimino)-2-methyl-3,11-dihydro-4H-anthraquinone[2,3-d]imidazole-4-one

¹H NMR (400 MHZ, DMSO-d₆) δ 13.01 (s, 1H), 8.78 (d, J=33.2 Hz, 2H), 8.15 (dd, J=16.8, 7.8 Hz, 2H), 7.65 (d, J=19.9 Hz, 2H), 7.43-7.12 (m, 5H), 5.83 (s, 2H), 2.65 (s, 3H).

HRMS (ESI): m/z theoretical value: C₁₂H₉N₃O₂ [M+H]⁺368.1394, measured value: 368.1395.

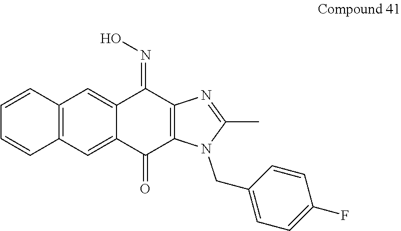 [SMILES: Cc1nc2c(n1Cc1ccc(F)cc1)C(=O)c1cc3ccccc3cc1/C2=N\O]

(E)-3-(4-fluorobenzyl)-11-(hydroxylimino)-2-methyl-3,11-dihydro-4H-anthraquinone[2,3-d]imidazole-4-one

¹H NMR (400 MHZ, DMSO-d₆) δ 13.50 (s, 1H), 8.81 (d, J=16.8 Hz, 2H), 8.24-8.13 (m, 2H), 7.75-7.60 (m, 2H), 7.16 (d, J=8.8 Hz, 4H), 5.86 (s, 2H), 2.62 (s, 3H).

HRMS (ESI): m/z theoretical value: C₁₂H₉N₃O₂ [M+H]⁺386.1300, measured value: 386.1327.

### Example 5

2,3-Diamine-1,4-benzoquinone (4 g) was dissolved into 13 mL of dimethyl sulfoxide, added with butyraldehyde (2.12 mL), and subjected to open-ended reaction at 80° C. for 13 h, and cooled to room temperature, then slowly added with water, solid was separated out, subjected to suction filtration and vacuum dried to obtain yellow solid 1,4-naphthoquinone imidazole (0.15 g). 1,4-naphthoquinone imidazole (0.3 g) and potassium hydroxide (0.15 g) were added to dimethyl sulfoxide solution (3 mL), respectively; the mixture was stirred at 50° C. for 30 min and added with iodobutane (0.2 mL), after reaction for 2, water was added to separate out solid; the solid was subjected to suction filtration, washed with water, and vacuum dried to obtain yellow solid 2-methyl-1,4-naphthoquinone imidazole (0.31 g). 2-methylnaphthoquinone imidazole (0.2 g), hydroxylamine hydrochloride (0.11 g), and pyridine (0.35 mL) were dissolved into ethanol (35 mL) and reacted for 36 h at 105° C.; at the end of reaction, solid was separated out from solution, directly subjected to suction filtration, and vacuum dried to obtain light yellow solid (0.15 g).

2,3, -Diamine-1,4-benzoquinone (4 g) was dissolved into 13 mL of dimethyl sulfoxide and reacted with different aldehydes; or 2,3, -diamine-1,4-naphthoquinone (20 g) was dissolved into dimethyl sulfoxide and reacted with different aldehydes; or halohydrocarbons having different substitutes were added to prepare Compounds 42-59 according to the method in Example 5; other materials and reaction conditions for the compounds were kept the same and raw materials could be purchased commercially:

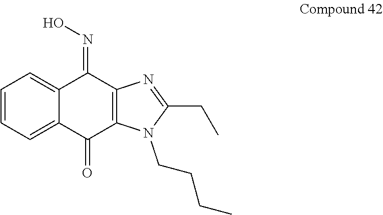 [SMILES: CCCCn1c(CC)nc2c1C(=O)c1ccccc1/C2=N\O]

(E)-3-butyl-9-(hydroxylimino)-2-ethyl-3,9-dihydro-4H-naphthol[2,3-d]imidazole-4-one

¹H NMR (400 MHZ, DMSO-d₆) δ 13.26 (s, 1H), 8.25 (d, J=7.9 Hz, 1H), 8.10 (d, J=8.0 Hz, 1H), 7.75-7.65 (m, 1H), 7.60 (t, J=7.5 Hz, 1H), 4.38 (t, J=7.5 Hz, 2H), 2.86 (q, J=7.5 Hz, 2H), 1.67 (ddd, J=12.6, 10.2, 6.5 Hz, 2H), 1.31 (td, J=7.5, 3.7 Hz, 5H), 0.89 (t, J=7.4 Hz, 3H).

HRMS (ESI): m/z theoretical value: C₁₂H₉N₃O₂ [M+H]⁺298.1550, measured value: 298.1557.

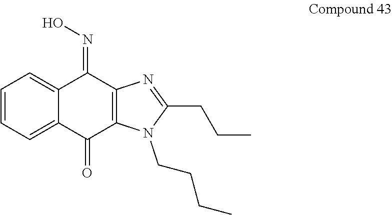 [SMILES: CCCCn1c(CCC)nc2c1C(=O)c1ccccc1/C2=N\O]

(E)-3-butyl-9-(hydroxylimino)-2-propyl-3,9-dihydro-4H-naphthol[2,3-d]imidazole-4-one

¹H NMR (400 MHZ, Chloroform-d) § 13.95 (s, 1H), 8.42 (d, J=7.8 Hz, 1H), 8.28-8.21 (m, 1H), 7.65 (t, J=7.3 Hz, 1H), 7.59-7.55 (m, 1H), 4.46 (t, J=7.6 Hz, 2H), 2.80 (t, J=7.6 Hz, 2H), 1.93 (h, J=7.7 Hz, 2H), 1.79 (q, J=7.7 Hz, 2H), 1.44 (q, J=7.6 Hz, 2H), 1.08 (t, J=7.4 Hz, 3H), 0.99 (t, J=7.4 Hz, 3H).

HRMS (ESI): m/z theoretical value: C₁₂H₉N₃O₂ [M+H]⁺312.1707, measured value: 312.1709.

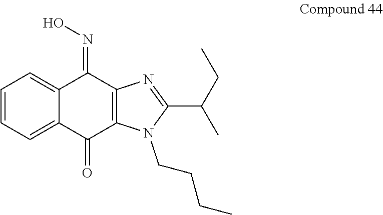 [SMILES: CCCCn1c(C(C)CC)nc2c1C(=O)c1ccccc1/C2=N\O]

(E)-2-(sec-butyl)-3-butyl-9-(hydroxylimino)-3,9-dihydro-4H-naphthol[2,3-d]imidazole-4-one

¹H NMR (400 MHZ, DMSO-d₆) δ 13.32 (s, 1H), 8.26 (d, J=8.0 Hz, 1H), 8.13 (d, J=7.8 Hz, 1H), 7.70 (t, J=7.7 Hz, 1H), 7.62 (t, J=7.5 Hz, 1H), 4.52-4.40 (m, 2H), 3.08 (q, J=6.8 Hz, 1H), 1.80 (dt, J=14.0, 7.4 Hz, 1H), 1.67 (q, J=7.3 Hz, 3H), 1.39-1.32 (m, 2H), 1.28 (d, J=6.8 Hz, 3H), 0.90 (t, J=7.4 Hz, 3H), 0.83 (t, J=7.2 Hz, 3H).

HRMS (ESI): m/z theoretical value: C₁₂H₉N₃O₂ [M+H]⁺326.1863, measured value: 326.1865.

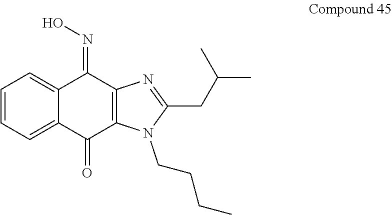 [SMILES: CCCCn1c(CC(C)C)nc2c1C(=O)c1ccccc1/C2=N\O]

(E)-3-butyl-9-(hydroxylimino)-2-isobutyl-3,9-dihydro-4H-naphthol[2,3-d]imidazole-4-one

¹H NMR (400 MHZ, Chloroform-d) & 13.91 (s, 1H), 8.38 (d, J=7.8 Hz, 1H), 8.21 (d, J=7.8 Hz, 1H), 7.62 (t, J=7.6 Hz, 1H), 7.54 (t, J=7.6 Hz, 1H), 4.44 (t, J=7.8 Hz, 2H), 2.68 (d, J=7.0 Hz, 2H), 2.32 (dp, J=13.8, 6.8 Hz, 1H), 1.78 (p, J=7.6 Hz, 2H), 1.44 (p, J=7.4 Hz, 2H), 1.11-0.91 (m, 9H).

HRMS (ESI): m/z theoretical value: C₁₂H₉N₃O₂ [M+H]⁺326.1863, measured value: 326.1862.

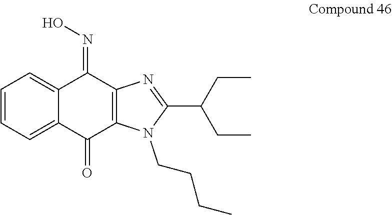 [SMILES: CCCCn1c(C(CC)CC)nc2c1C(=O)c1ccccc1/C2=N\O]

(E)-3-butyl-9-(hydroxylimino)-2-(pentane-3-yl)-3,9-dihydro-4H-naphthol[2,3-d]

¹H NMR (400 MHZ, Chloroform-d) § 14.05 (s, 1H), 8.44 (dd, J=8.0, 1.2 Hz, 1H), 8.29-8.20 (m, 1H), 7.67 (td, J=7.6, 1.6 Hz, 1H), 7.59 (td, J=7.6, 1.4 Hz, 1H), 4.57-4.46 (m, 2H), 2.79 (tt, J=8.4, 5.6 Hz, 1H), 1.92-1.80 (m, 6H), 1.49 (h, J=7.4 Hz, 2H), 1.02 (t, J=7.4 Hz, 3H), 0.90 (t, J=7.4 Hz, 6H).

HRMS (ESI): m/z theoretical value: C₁₂H₉N₃O₂ [M+H]⁺340.2020, measured value: 340.2025.

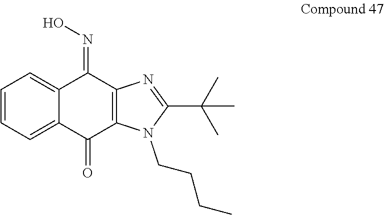 [SMILES: CCCCn1c(C(C)(C)C)nc2c1C(=O)c1ccccc1/C2=N\O]

(E)-3-butyl-9-(hydroxylimino)-2-tert-butyl-3,9-dihydro-4H-naphthol imidazole-4-one[2,3-d]

¹H NMR (400 MHZ, Chloroform-d) & 14.09 (s, 1H), 8.40 (d, J=7.8 Hz, 1H), 8.25 (d, J=7.8 Hz, 1H), 7.64 (t, J=7.2 Hz, 1H), 7.56 (t, J=7.8 Hz, 1H), 4.62-4.49 (m, 2H), 1.95-1.85 (m, 2H), 1.60-1.53 (m, 11H), 1.03 (t, J=7.2 Hz, 3H).

HRMS (ESI): m/z theoretical value: C₁₂H₉N₃O₂ [M+H]⁺326.1863, measured value: 326.1864.

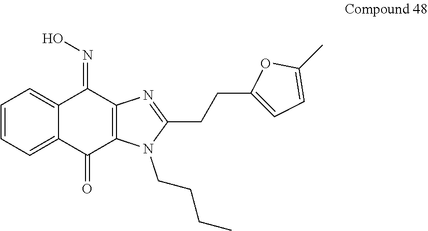 [SMILES: CCCCn1c(CCc2ccc(C)o2)nc2c1C(=O)c1ccccc1/C2=N\O]

(E)-3-butyl-9-(hydroxylimino)-2-(2-(5-methylfuran-2-yl)ethyl)-3,9-dihydro-4H-naphthol[2,3-d]imidazole-4-one

¹H NMR (400 MHZ, Chloroform-d) & 13.87 (s, 1H), 8.42 (d, J=7.8 Hz, 1H), 8.24 (d, J=7.8 Hz, 1H), 7.67-7.62 (m, 1H), 7.60-7.53 (m, 1H), 5.91 (d, J=3.0 Hz, 1H), 5.88-5.81 (m, 1H), 4.38 (t, J=7.6 Hz, 2H), 3.24-3.07 (m, 3H), 2.25 (s, 3H), 1.90-1.83 (m, 1H), 1.78-1.67 (m, 2H), 1.41 (q, J=7.6 Hz, 2H), 0.97 (t, J=7.2 Hz, 3H).

HRMS (ESI): m/z theoretical value: C₁₂H₉N₃O₂ [M+H]⁺378.1812, measured value: 378.1813.

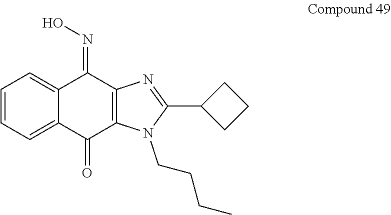 [SMILES: CCCCn1c(C2CCC2)nc2c1C(=O)c1ccccc1/C2=N\O]

(E)-3-butyl-9-(hydroxylimino)-2-cyclobutyl-3,9-dihydro-4H-naphthol imidazole-4-one[2,3-d]

¹H NMR (400 MHZ, Chloroform-d) & 14.08 (s, 1H), 8.42 (d, J=7.8 Hz, 1H), 8.29-8.19 (m, 1H), 7.65 (td, J=8.2, 7.8, 1.6 Hz, 1H), 7.60-7.51 (m, 1H), 4.44-4.28 (m, 2H), 3.68 (q, J=8.5 Hz, 1H), 2.62-2.52 (m, 2H), 2.44 (q, J=8.9 Hz, 2H), 2.17 (q, J=9.5 Hz, 1H), 2.09-2.04 (m, 1H), 1.76 (p, J=7.8 Hz, 2H), 1.48-1.35 (m, 2H), 0.98 (t, J=7.3 Hz, 3H).

HRMS (ESI): m/z theoretical value: C₁₂H₉N₃O₂ [M+H]⁺324.1707, measured value: 324.1709.

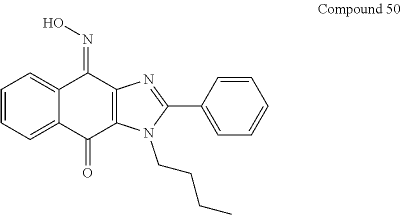 [SMILES: CCCCn1c(-c2ccccc2)nc2c1C(=O)c1ccccc1/C2=N\O]

(E)-3-butyl-9-(hydroxylimino)-2-phenyl-3,9-dihydro-4H-naphthol[2,3-d]imidazole-4-one

¹H NMR (400 MHZ, Chloroform-d) & 13.92 (s, 1H), 8.46 (d, J=7.8 Hz, 1H), 8.29 (d, J=7.8 Hz, 1H), 7.69 (d, J=7.4 Hz, 2H), 7.63-7.58 (m, 5H), 4.58 (t, J=7.8 Hz, 2H), 1.86 (q, J=7.6 Hz, 2H), 1.37-1.31 (m, 2H), 0.89 (t, J=7.2 Hz, 3H).

HRMS (ESI): m/z theoretical value: C₁₂H₉N₃O₂ [M+H]⁺346.1550, measured value: 346.1554.

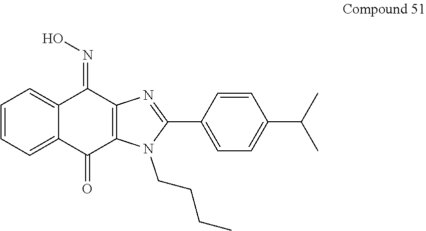 [SMILES: CCCCn1c(-c2ccc(C(C)C)cc2)nc2c1C(=O)c1ccccc1/C2=N\O]

(E)-3-butyl-9-(hydroxylimino)-2-(4-isopropylphenyl)-3,9-dihydro-4H-naphthol[2,3-d]imidazole-4-one

¹H NMR (400 MHZ, Chloroform-d) & 13.96 (s, 1H), 8.44 (d, J=7.8 Hz, 1H), 8.28 (dd, J=78, 1.6 Hz, 1H), 7.93-7.54 (m, 4H), 7.52-7.31 (m, 2H), 4.57 (t, J=7.8 Hz, 2H), 1.92-1.83 (m, 2H), 1.42-1.25 (m, 8H), 0.90 (t, J=7.4 Hz, 3H).

HRMS (ESI): m/z theoretical value: C₁₂H₉N₃O₂ [M+H]⁺388.2020, measured value: 388.2024.

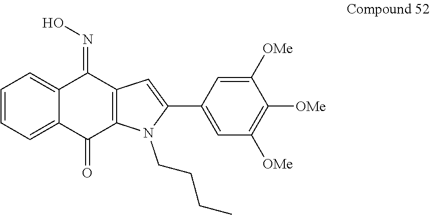 [SMILES: CCCCn1c(-c2cc(OC)c(OC)c(OC)c2)cc2c1C(=O)c1ccccc1/C2=N\O]

(E)-3-butyl-9-(hydroxylimino)-2-(3,4,5-trimethoxyphenyl)-3,9-dihydro-4H-naphthol[2,3-d]imidazole-4-one

¹H NMR (400 MHZ, Chloroform-d) & 13.85 (s, 1H), 8.45 (d, J=7.8 Hz, 1H), 8.29 (d, J=7.8 Hz, 1H), 7.68 (t, J=8.0 Hz, 1H), 7.60 (t, J=7.4 Hz, 1H), 6.88 (s, 2H), 4.63-4.52 (m, 2H), 3.94 (s, 9H), 1.92-1.86 (m, 2H), 1.40-1.34 (m, 2H), 0.93 (t, J=7.4 Hz, 3H).

HRMS (ESI): m/z theoretical value: C₁₂H₉N₃O₂ [M+H]⁺436.1867, measured value: 436.1868.

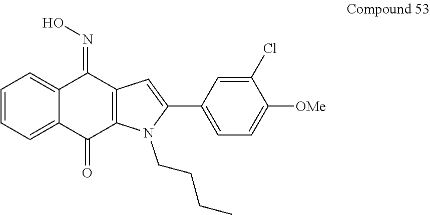 [SMILES: CCCCn1c(-c2ccc(OC)c(Cl)c2)cc2c1C(=O)c1ccccc1/C2=N\O]

(E)-3-butyl-2-(3-chloro-4-methoxyphenyl)-9-(hydroxylimino)-3,9-dihydro-4H-naphthol[2,3-d]imidazole-4-one

¹H NMR (400 MHZ, DMSO-d₆) δ 13.10 (s, 1H), 8.30 (d, J=8.0 Hz, 1H), 8.16 (d, J=7.6 Hz, 1H), 7.81 (s, 1H), 7.70 (d, J=8.4 Hz, 2H), 7.64 (d, J=7.6 Hz, 1H), 7.34 (d, J=8.5 Hz, 1H), 4.48 (t, J=8.4 Hz, 2H), 3.94 (s, 3H), 1.72-1.67 (m, 2H), 1.19-1.12 (m, 2H), 0.76 (t, J=7.4 Hz, 3H).

HRMS (ESI): m/z theoretical value: C₁₂H₉N₃O₂ [M+H]⁺410.1266, measured value: 410.1266.

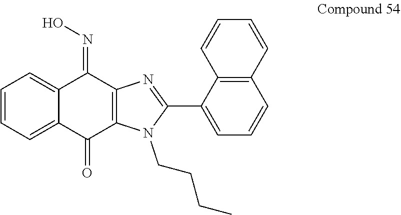 [SMILES: CCCCn1c(-c2cccc3ccccc23)nc2c1C(=O)c1ccccc1/C2=N\O]

(E)-3-butyl-9-(hydroxylimino)-2-naphthyl-3,9-dihydro-4H-naphthol[2,3-d]imidazole-4-one

¹H NMR (400 MHZ, Chloroform-d) § 13.83 (s, 1H), 8.50 (dd, J=7.8, 1.2 Hz, 1H), 8.34 (dd, J=7.8, 1.6 Hz, 1H), 8.12 (dd, J=7.2, 2.2 Hz, 1H), 8.01 (dd, J=8.0, 1.8 Hz, 1H), 7.78-7.51 (m, 7H), 4.38 (t, J=7.4 Hz, 2H), 1.67 (p, J=7.6 Hz, 2H), 1.13 (h, J=7.4 Hz, 2H), 0.69 (t, J=7.4 Hz, 3H).

HRMS (ESI): m/z theoretical value: C₁₂H₉N₃O₂ [M+H]⁺396.1707, measured value: 396.1709.

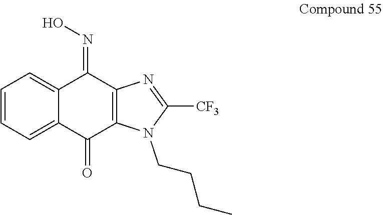 [SMILES: CCCCn1c(C(F)(F)F)nc2c1C(=O)c1ccccc1/C2=N\O]

(E)-3-butyl-9-(hydroxylimino)-2-(trifluoromethyl)-3,9-dihydro-4H-naphthol[2,3-d]imidazole-4-one

¹H NMR (400 MHZ, Chloroform-d) § 12.85 (s, 1H), 8.41 (dd, J=8.0, 1.2 Hz, 1H), 8.24 (dd, J=7.8, 1.6 Hz, 1H), 7.69 (td, J=7.8, 1.6 Hz, 1H), 7.59 (td, J=7.6, 1.2 Hz, 1H), 4.72-4.54 (m, 2H), 1.88 (p, J=8.0 Hz, 2H), 1.50 (h, J=7.4 Hz, 2H), 1.01 (t, J=7.4 Hz, 3H).

HRMS (ESI): m/z theoretical value: C₁₂H₉N₃O₂ [M+H]⁺338.1111, measured value: 338.1115.

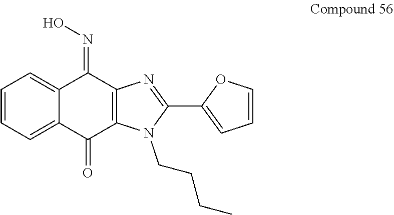 [SMILES: CCCCn1c(-c2ccco2)nc2c1C(=O)c1ccccc1/C2=N\O]

(E)-3-butyl-9-(hydroxylimino)-2-(furan-2-yl)-3,9-dihydro-4H-naphthol imidazole-4-one[2,3-d]

¹H NMR (400 MHZ, Chloroform-d) § 13.80 (s, 1H), 8.43 (d, J=8.0 Hz, 1H), 8.28 (d, J=7.8 Hz, 1H), 7.66 (d, J=10.2 Hz, 2H), 7.61-7.55 (m, 1H), 7.24 (d, J=4.8 Hz, 1H), 4.96-4.89 (m, 2H), 1.89 (q, J=7.6 Hz, 2H), 1.47 (q, J=7.6 Hz, 2H), 0.99 (t, J=7.2 Hz, 3H).

HRMS (ESI): m/z theoretical value: C₁₂H₉N₃O₂ [M+H]⁺336.1343, measured value: 336.1344.

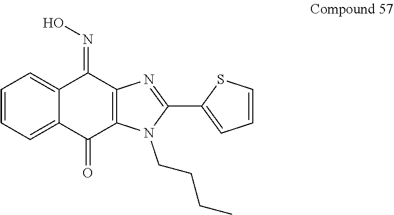 [SMILES: CCCCn1c(-c2cccs2)nc2c1C(=O)c1ccccc1/C2=N\O]

(E)-3-butyl-9-(hydroxylimino)-2-(thiophene-2-yl)-3,9-dihydro-4H-naphthol[2,3-d]imidazole-4-one

¹H NMR (400 MHZ, Chloroform-d) § 13.78 (s, 1H), 8.44 (d, J=8.0 Hz, 1H), 8.34-8.13 (m, 1H), 7.70-7.52 (m, 4H), 7.24 (s, 1H), 4.82-4.71 (m, 2H), 1.95 (p, J=8.2 Hz, 2H), 1.53-1.45 (m, 2H), 1.01 (t, J=7.4 Hz, 3H).

HRMS (ESI): m/z theoretical value: C₁₂H₉N₃O₂ [M+H]⁺352.1114, measured value: 352.1116.

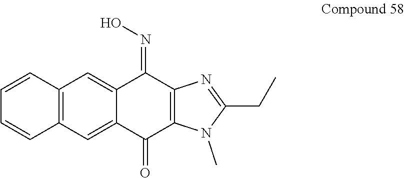 [SMILES: CCc1nc2c(n1C)C(=O)c1cc3ccccc3cc1/C2=N\O]

(E)-2-ethyl-11-(hydroxylimino)-3-methyl-3,11-dihydro-4H-anthraquinone[2,3-d]imidazole-4-one

¹H NMR (400 MHZ, DMSO-d₆) δ 13.71 (s, 1H), 8.60 (d, J=5.6 Hz, 2H), 8.21-8.15 (m, 2H), 7.71-7.65 (m, 2H), 2.76 (q, J=7.6 Hz, 2H), 1.27 (t, J=7.6 Hz, 2H), 4.09 (s, 1H).

HRMS (ESI): m/z theoretical value: C₁₂H₉N₃O₂ [M+H]⁺306.1237, measured value: 306.1236.

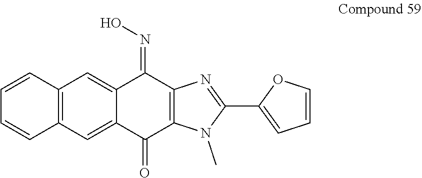 [SMILES: Cn1c(-c2ccco2)nc2c1C(=O)c1cc3ccccc3cc1/C2=N\O]

(E)-2-(furan-2-yl)-11-(hydroxylimino)-3-methyl-3,11-dihydro-4H-anthraquinone[2,3-d]imidazole-4-one

¹H NMR (400 MHZ, DMSO-d₆) δ 13.85 (s, 1H), 8.69 (s, 2H), 8.27-8.22 (m, 2H), 7.95 (d, J=1.2 Hz, 1H), 7.76-7.70 (m, 2H), 7.35 (d, J=3.4 Hz, 1H), 6.75-6.70 (m, 1H), 4.25 (s, 1H).

HRMS (ESI): m/z theoretical value: C₁₂H₉N₃O₂ [M+H]⁺344.1030, measured value: 344.1031.

### Example 6

2,3-Diamine-1,4-benzoquinone (20 g) was dissolved into 100 mL of formic acid, subjected to reflux reaction for 6 h, and cooled to room temperature, then poured into an ice-water mixture with the addition of ammonium hydroxide to regulate pH to 9-10, and the obtained solution was subjected to suction filtration and vacuum dried to obtain yellow solid 1,4-naphthoquinone imidazole (22 g). 1,4-naphthoquinone imidazole (0.3 g) and potassium hydroxide (0.15 g) were added to dimethyl sulfoxide solution (3 mL); the mixture was stirred at 50° C. for 30 min and added with iodomethane (0.2 mL), after reaction for 2, water was added to separate out solid; the solid was subjected to suction filtration, washed with water, and vacuum dried to obtain yellow solid 2-methyl-1,4-naphthoquinone imidazole (0.31 g). 2-methylnaphthoquinone imidazole (0.2 g), methoxyamine hydrochloride (0.1 g), and pyridine (0.08 mL) were dissolved into ethanol (30 mL) and reacted for 12 h at 125° C.; at the end of reaction, solid was separated out from solution, directly subjected to suction filtration, and vacuum dried to obtain light yellow solid (0.15 g).

2,3-Diamine-1,4-naphthoquinone (20 g) was dissolved into 100 mL of formic acid and subjected to reflux reaction for 6 h to obtain anthraquinone imidazole; or aminoxyhydrochlorides having different substitutes were added to prepare Compounds 60-77 according to the method in Example 6; other materials and reaction conditions for the compounds were kept the same and raw materials could be purchased commercially:

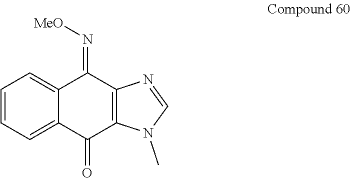 [SMILES: CO/N=C1\c2ccccc2C(=O)c2c1ncn2C]

(E) and (Z)-9-(methoxyimino)-3-methyl-3,9-dihydro-4H-naphthol[2,3-d]imidazole-4-one

¹H NMR (400 MHZ, DMSO-d₆) δ 8.99 (dd, J=8.0, 1.2 Hz, 1H), 8.31-8.20 (m, 4H), 8.16-8.07 (m, 2H), 7.82-7.60 (m, 5H), 4.24 (s, 3H), 4.20 (s, 4H), 4.03 (s, 4H), 3.99 (s, 3H).

¹³C NMR (101 MHz, DMSO) δ 175.40, 174.52, 145.91, 145.85, 145.58, 140.34, 139.95, 139.03, 133.69, 133.20, 133.12, 132.39, 131.62, 131.20, 131.09, 129.94, 128.10, 128.03, 127.04, 126.43, 126.16, 124.33, 64.73, 64.47, 34.20, 33.92.

HRMS (ESI): m/z theoretical value: C₁₃H₁₁N₃O₂ [M+H]⁺242.0924, measured value: 242.0928.

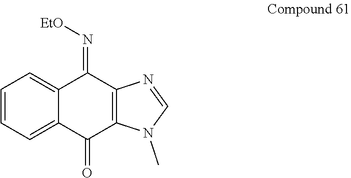 [SMILES: CCO/N=C1\c2ccccc2C(=O)c2c1ncn2C]

(E) and (Z)-9-(ethoxyimino)-3-methyl-3,9-dihydro-4H-naphthol[2,3-d]imidazole-4-one

¹H NMR (400 MHZ, DMSO-d₆) δ 9.04 (dd, J=8.0, 1.2 Hz, 1H), 8.30 (dd, J=8.0, 1.2 Hz, 2H), 8.26-8.20 (m, 3H), 8.14-8.07 (m, 3H), 7.79 (td, J=8.0, 1.6 Hz, 1H), 7.75-7.68 (m, 3H), 7.63 (td, J=8.0, 1.2 Hz, 2H), 4.53-4.42 (m, 6H), 4.04 (s, 6H), 4.00 (s, 3H), 1.46-1.32 (m, 9H).

¹³C NMR (101 MHz, DMSO) δ 175.42, 174.53, 146.10, 145.90, 145.64, 140.16, 139.75, 139.33, 133.66, 133.50, 133.04, 132.38, 131.51, 131.07, 129.79, 128.08, 128.04, 127.00, 126.37, 126.10, 124.33, 124.09, 72.72, 72.20, 34.16, 33.90, 15.29, 15.04.

HRMS (ESI): m/z theoretical value: C₁₄H₁₃N₃O₂ [M+H]⁺256.1081, measured value: 256.1085.

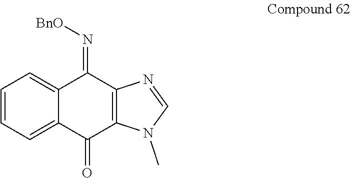 [SMILES: Cn1cnc2c1C(=O)c1ccccc1/C2=N\OCc1ccccc1]

(E) and (Z)-9-(benzyloxyimino)-3-methyl-3,9-dihydro-4H-naphthol[2,3-d]imidazole-4-one

¹H NMR (400 MHZ, DMSO-d₆) δ 8.30-8.22 (m, 2H), 8.11 (dd, J=8.0, 1.2 Hz, 1H), 7.71 (td, J=8.0, 1.6 Hz, 1H), 7.64 (td, J=8.0, 1.2 Hz, 1H), 7.55-7.49 (m, 2H), 7.45-7.31 (m, 3H), 5.50 (s, 2H), 4.03 (s, 3H).

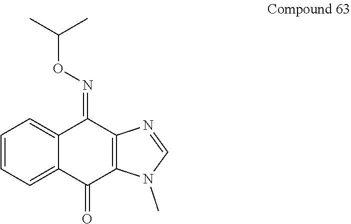 [SMILES: CC(C)O/N=C1\c2ccccc2C(=O)c2c1ncn2C]

(E) and (Z)-9-(isopropoxyimino)-3-methyl-3,9-dihydro-4H-naphthol[2,3-d]imidazole-4-one

¹H NMR (400 MHZ, DMSO-d₆) δ 9.07 (dd, J=8.0, 1.2 Hz, 1H), 8.33 (dd, J=8.0, 1.2 Hz, 1H), 8.28-8.21 (m, 2H), 8.16-8.10 (m, 2H), 7.80 (td, J=8.0, 1.6 Hz, 1H), 7.72 (td, J=8.0, 1.4 Hz, 2H), 7.63 (td, J=8.0, 1.2 Hz, 1H), 4.72-4.60 (m, 2H), 4.04 (s, 3H), 4.00 (s, 3H), 1.43 (dd, J=12.0, 8.0 Hz, 12H).

¹³C NMR (101 MHz, DMSO) δ 175.44, 174.53, 146.27, 145.91, 145.69, 139.80, 139.47, 139.28, 133.77, 133.66, 133.03, 132.39, 131.42, 131.07, 130.99, 129.68, 128.14, 128.00, 127.00, 126.32, 126.07, 124.33, 79.35, 78.63, 34.16, 33.91, 22.10, 21.97.

HRMS (ESI): m/z theoretical value: C₁₅H₁₅N₃O₂ [M+H]⁺270.1237, measured value: 270.1238.

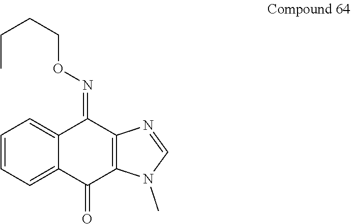 [SMILES: CCCCO/N=C1\c2ccccc2C(=O)c2c1ncn2C]

(E) and (Z)-9-(butoxyimino)-3-methyl-3,9-dihydro-4H-naphthol[2,3-d]imidazole-4-one

¹H NMR (400 MHZ, DMSO-d₆) δ 9.03 (dd, J=8.0, 1.2 Hz, 2H), 8.30 (dd, J=8.0, 1.2 Hz, 1H), 8.28-8.21 (m, 4H), 8.14 (s, 3H), 7.87-7.78 (m, 3H), 7.76-7.69 (m, 3H), 7.64 (td, J=8.0, 1.2 Hz, 1H), 4.48-4.38 (m, 7H), 4.04 (s, 3H), 4.00 (s, 7H), 1.85-1.72 (m, 7H), 1.53-1.39 (m, 7H), 1.00-0.92 (m, 10H).

¹³C NMR (101 MHZ, DMSO) δ 175.45, 174.54, 146.12, 145.96, 145.75, 140.23, 139.79, 139.45, 133.74, 133.53, 133.09, 132.41, 131.42, 131.14, 129.82, 128.11, 128.07, 127.07, 126.92, 126.40, 126.14, 124.34, 76.88, 76.36, 34.17, 33.92, 31.53, 31.20, 19.23, 19.08, 14.25, 14.23.

HRMS (ESI): m/z theoretical value: C₁₆H₁₇N₃O₂ [M+H]⁺284.1394, measured value: 284.1398.

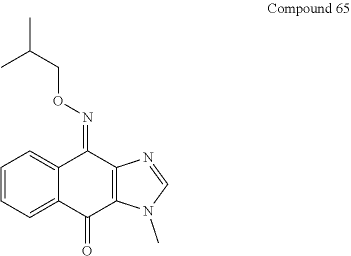 [SMILES: CC(C)CO/N=C1\c2ccccc2C(=O)c2c1ncn2C]

(E) and (Z)-9-(isobutoxyimino)-3-methyl-3,9-dihydro-4H-naphthol[2,3-d]imidazole-4-one

¹H NMR (400 MHZ, DMSO-d₆) δ 9.04 (dd, J=8.1, 1.2 Hz, 1H), 8.33-8.23 (m, 4H), 8.15-8.09 (m, 3H), 7.82 (td, J=8.0, 1.6 Hz, 1H), 7.75-7.69 (m, 2H), 7.64 (td, J=8.0, 1.2 Hz, 2H), 4.25 (d, J=8.0 Hz, 2H), 4.21 (d, J=8.0 Hz, 3H), 4.04 (s, 5H), 4.00 (s, 3H), 2.20-2.09 (m, 3H), 1.01 (t, J=6.0 Hz, 16H).

¹³C NMR (101 MHZ, DMSO) δ 175.44, 174.52, 146.10, 145.95, 145.77, 140.24, 139.84, 139.48, 133.73, 133.50, 133.07, 132.42, 131.34, 131.14, 131.10, 129.80, 128.08, 127.09, 126.40, 126.13, 124.32, 83.43, 82.87, 34.16, 33.92, 28.50, 28.21, 19.45, 19.42.

HRMS (ESI): m/z theoretical value: C₁₆H₁₇N₃O₂ [M+H]⁺284.1394, measured value: 284.1397.

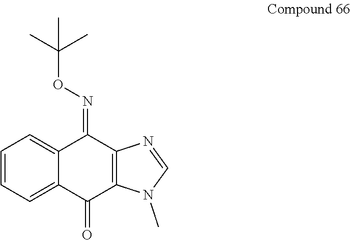 [SMILES: Cn1cnc2c1C(=O)c1ccccc1/C2=N\OC(C)(C)C]

(E) and (Z)-9-(tert-butoxyimino)-3-methyl-3,9-dihydro-4H-naphthol[2,3-d]imidazole-4-one

¹H NMR (400 MHZ, DMSO-d₆) δ 9.10 (dd, J=8.0, 1.2 Hz, 1H), 8.38 (dd, J=8.0, 1.2 Hz, 1H), 8.27-8.21 (m, 2H), 8.15-8.10 (m, 2H), 7.83-7.60 (m, 5H), 4.05 (s, 3H), 4.01 (s, 4H), 1.50 (s, 12H), 1.48 (s, 9H).

¹³C NMR (101 MHz, DMSO) δ 175.47, 174.56, 146.58, 145.91, 145.70, 139.61, 139.39, 138.81, 134.17, 133.61, 133.01, 132.40, 131.32, 131.05, 130.83, 129.52, 128.22, 127.95, 126.95, 126.23, 126.02, 124.29, 82.90, 82.08, 34.15, 33.90, 28.02, 27.74.

HRMS (ESI): m/z theoretical value: C₁₆H₁₇N₃O₂ [M+H]⁺284.1394, measured value: 284.1393.

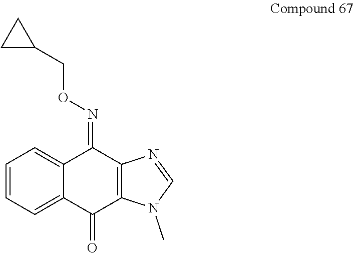 [SMILES: Cn1cnc2c1C(=O)c1ccccc1/C2=N\OCC1CC1]

(E) and (Z)-9-((cyclopropoxy)imino)-3-methyl-3,9-dihydro-4H-naphthol[2,3-d]

¹H NMR (400 MHz, DMSO-d₆) δ 9.10 (dd, J=8.0, 1.2 Hz, 1H), 8.33-8.24 (m, 4H), 8.16-8.09 (m, 3H), 7.84-7.77 (m, 1H), 7.75-7.70 (m, 2H), 7.64 (td, J=8.0, 1.2 Hz, 2H), 4.32-4.24 (m, 5H), 4.04 (s, 5H), 4.00 (s, 3H), 1.36-1.26 (m, 3H), 0.65-0.55 (m, 6H), 0.48-0.35 (m, 5H).

¹³C NMR (101 MHZ, DMSO) δ 175.45, 174.54, 146.15, 145.95, 145.72, 140.08, 139.62, 139.41, 133.69, 133.58, 133.07, 132.40, 131.50, 131.11, 131.09, 129.77, 128.17, 128.06, 127.04, 126.39, 126.12, 124.35, 81.62, 80.98, 34.16, 33.91, 11.04, 10.65, 3.64, 3.59.

HRMS (ESI): m/z theoretical value: C₁₆H₁₅N₃O₂ [M+H]⁺282.1237, measured value: 282.1238.

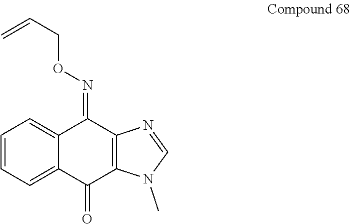 [SMILES: C=CCO/N=C1\c2ccccc2C(=O)c2c1ncn2C]

(E) and (Z)-9-((allyloxy)imino)-3-methyl-3,9-dihydro-4H-naphthol[2,3-d]imidazole-4-one

¹H NMR (400 MHZ, DMSO-d₆) δ 9.03 (dd, J=8.0, 1.2 Hz, 1H), 8.30-8.21 (m, 6H), 8.15-8.08 (m, 4H), 7.80 (td, J=8.0, 1.6 Hz, 1H), 7.77-7.65 (m, 4H), 7.64 (td, J=8.0, 1.2 Hz, 3H), 6.22-6.08 (m, 4H), 5.55-5.40 (m, 4H), 5.36-5.27 (m, 4H), 5.00-4.92 (m, 7H), 4.04 (s, 8H), 3.99 (s, 3H).

¹³C NMR (101 MHZ, DMSO) δ 175.43, 174.51, 145.94, 145.72, 140.65, 140.24, 139.31, 135.14, 134.58, 134.13, 133.71, 133.34, 133.09, 132.42, 131.59, 131.22, 131.12, 129.93, 128.15, 128.05, 127.08, 126.48, 126.15, 124.37, 119.04, 118.54, 77.72, 77.22, 34.16, 33.92.

HRMS (ESI): m/z theoretical value: C₁₅H₁₃N₃O₂ [M+H]⁺268.1081, measured value: 268.1083.

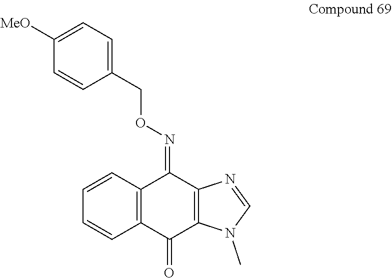 [SMILES: COc1ccc(CO/N=C2\c3ccccc3C(=O)c3c2ncn3C)cc1]

(E) and (Z)-3-methyl-9-(((4-methoxybenzyl)oxy)imino)-3,9-dihydro-4H-naphthol[2,3-d]imidazole-4-one

¹H NMR (400 MHZ, DMSO-d₆) δ 9.05-8.98 (m, 1H), 8.34-8.27 (m, 3H), 8.27-8.20 (m, 4H), 8.15-8.09 (m, 4H), 7.76-7.61 (m, 8H), 7.49-7.43 (m, 8H), 6.99-6.93 (m, 8H), 5.44 (s, 2H), 5.41 (s, 6H), 4.03 (s, 9H), 3.99 (s, 3H), 3.76 (s, 3H), 3.75 (s, 9H).

¹³C NMR (101 MHz, DMSO) δ 175.43, 174.52, 159.81, 159.60, 145.98, 145.75, 140.52, 140.16, 139.34, 133.72, 133.41, 133.13, 132.42, 131.50, 131.19, 131.13, 130.65, 130.54, 129.93, 129.82, 129.17, 128.15, 128.09, 127.08, 126.48, 126.16, 124.38, 114.40, 114.24, 78.57, 78.04, 55.58, 55.55, 34.16, 33.92.

HRMS (ESI): m/z theoretical value: C₂₀H₁₇N₃O₃ [M+H]⁺348.1343, measured value: 348.1339.

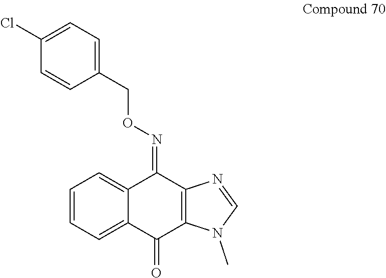 [SMILES: Cn1cnc2c1C(=O)c1ccccc1/C2=N\OCc1ccc(Cl)cc1]

(E) and (Z)-3-methyl-9-(((4-chlorobenzyl)oxy)imino)-3,9-dihydro-4H-naphthol[2,3-d]imidazole-4-one

¹H NMR (400 MHZ, DMSO-d₆) δ 9.03 (dd, J=8.0, 1.2 Hz, 1H), 8.28-8.22 (m, 5H), 8.14-8.08 (m, 3H), 7.82-7.60 (m, 7H), 7.57-7.43 (m, 13H), 5.51 (s, 2H), 5.49 (s, 4H), 4.03 (s, 6H), 3.99 (s, 3H).

¹³C NMR (101 MHz, DMSO) δ 175.42, 174.50, 145.97, 145.80, 140.99, 140.68, 139.26, 137.10, 136.54, 133.77, 133.30, 133.21, 133.16, 132.98, 132.47, 131.63, 131.33, 131.31, 131.16, 130.52, 130.32, 130.06, 129.03, 128.87, 128.27, 128.03, 127.13, 126.55, 126.20, 124.40, 77.67, 77.17, 34.17, 33.92.

HRMS (ESI): m/z theoretical value: C₁₉H₁₄ClN₃O₂ [M+H]⁺352.0847, measured value: 352.0843.

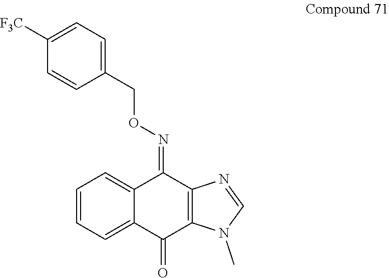 [SMILES: Cn1cnc2c1C(=O)c1ccccc1/C2=N\OCc1ccc(C(F)(F)F)cc1]

(E) and (Z)-3-methyl-9-(((4-trifluoromethyl benzyl)oxy)imino)-3,9-dihydro-4H-naphthol[2,3-d]imidazole-4-one yellow solid, 100 mg, % yield, m. p. 136-138° C.

¹H NMR (400 MHZ, DMSO-d₆) δ 9.07 (dd, J=8.0, 1.2 Hz, 1H), 8.28-8.21 (m, 4H), 8.14-8.07 (m, 3H), 7.82-7.60 (m, 17H), 5.63 (s, 2H), 5.60 (s, 3H), 4.04 (s, 5H), 3.99 (s, 3H).

¹³C NMR (101 MHz, DMSO) δ 175.40, 174.48, 145.95, 145.80, 145.73, 142.98, 142.48, 141.24, 140.95, 139.23, 133.78, 133.15, 133.11, 132.48, 131.70, 131.35, 131.16, 130.09, 128.94, 128.74, 128.62, 128.31, 128.00, 127.13, 126.57, 126.19, 126.07, 125.95, 125.91, 125.87, 125.83, 125.79, 125.76, 125.72, 125.68, 124.39, 123.37, 77.49, 77.04, 34.17, 33.90.

HRMS (ESI): m/z theoretical value: C₂₀H₁₄F₃N₃O₂ [M+H]⁺386.1111, measured value: 386.1107.

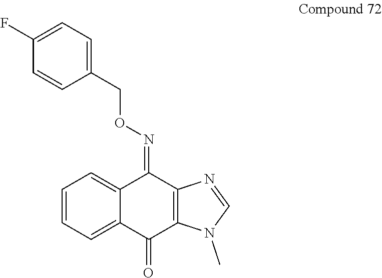 [SMILES: Cn1cnc2c1C(=O)c1ccccc1/C2=N\OCc1ccc(F)cc1]

(E) and (Z)-3-methyl-9-(((4-fluorobenzyl)oxy)imino)-3,9-dihydro-4H-naphthol[2,3-d]imidazole-4-one

¹H NMR (400 MHz, DMSO-d₆) δ 9.03 (dd, J=8.0, 1.4 Hz, 1H), 8.33-8.20 (m, 5H), 8.18-8.09 (m, 3H), 7.79-7.53 (m, 12H), 7.30-7.17 (m, 7H), 5.50 (s, 2H), 5.48 (s, 3H), 4.03 (s, 5H), 3.99 (s, 3H).

¹³C NMR (101 MHZ, DMSO) δ 175.43, 174.51, 163.71, 163.54, 161.12, 145.98, 145.88, 145.79, 140.85, 140.53, 139.28, 134.26, 134.23, 133.76, 133.72, 133.69, 133.27, 133.16, 132.46, 131.59, 131.28, 131.16, 131.06, 130.98, 130.84, 130.76, 130.03, 129.51, 128.24, 128.04, 127.12, 126.54, 126.19, 124.40, 115.96, 115.79, 115.75, 115.57, 77.83, 77.35, 34.17, 33.92.

HRMS (ESI): m/z theoretical value: C₁₉H₁₄FN₃O₂ [M+H]⁺336.1143, measured value: 336.1140.

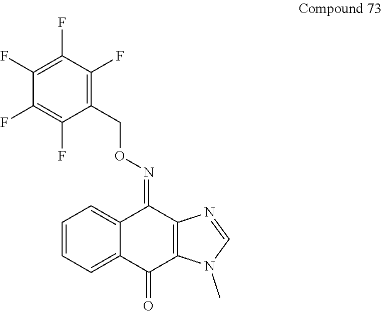 [SMILES: Cn1cnc2c1C(=O)c1ccccc1/C2=N\OCc1c(F)c(F)c(F)c(F)c1F]

(E) and (Z)-3-methyl-9-(((perfluorobenzyl)oxy)imino)-3,9-dihydro-4H-naphthol[2,3-d]imidazole-4-one yellow solid, 100 mg, % yield, m. p. 224-226° C.

¹H NMR (400 MHZ, DMSO-d₆) δ 8.92 (dd, J=8.0, 1.2 Hz, 1H), 8.26-8.07 (m, 7H), 7.83-7.71 (m, 4H), 7.65 (td, J=8.0, 1.2 Hz, 2H), 5.61 (s, 2H), 5.53 (s, 3H), 4.02 (s, 5H), 3.98 (s, 3H).

¹³C NMR (101 MHZ, DMSO) δ 175.39, 174.49, 146.06, 145.82, 145.43, 144.63, 141.75, 141.24, 138.94, 133.86, 133.81, 133.32, 132.90, 132.55, 131.67, 131.67, 131.59, 131.23, 130.34, 130.11, 128.49, 128.46, 127.93, 127.87, 127.20, 126.26, 124.69, 124.25, 65.48, 64.75, 34.17, 33.93.

HRMS (ESI): m/z theoretical value: C₁₉H₁₀F₅N₃O₂ [M+H]⁺408.0766, measured value: 408.0767.

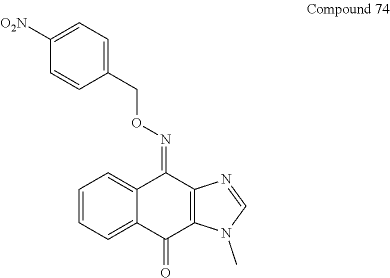 [SMILES: Cn1cnc2c1C(=O)c1ccccc1/C2=N\OCc1ccc([N+](=O)[O-])cc1]

(E) and (Z)-3-methyl-9-(((4-nitrobenzyl)oxy)imino)-3,9-dihydro-4H-naphthol[2,3-d]imidazole-4-one

¹H NMR (400 MHZ, DMSO-d₆) δ 9.09 (dd, J=8.0, 1.2 Hz, 1H), 8.31-8.17 (m, 14H), 8.13-8.09 (m, 3H), 7.84-7.62 (m, 15H), 5.67 (s, 2H), 5.65 (s, 5H), 4.08-4.02 (m, 8H), 3.99-3.97 (m, 3H).

¹³C NMR (101 MHZ, DMSO) δ 175.42, 174.50, 147.64, 147.47, 146.09, 145.97, 145.85, 145.66, 145.55, 141.45, 141.20, 139.20, 133.85, 133.21, 133.02, 132.51, 131.79, 131.46, 131.18, 130.20, 129.18, 128.95, 128.37, 127.98, 127.19, 126.62, 126.24, 124.43, 124.15, 124.03, 77.09, 76.65, 34.20, 33.93.

HRMS (ESI): m/z theoretical value: C₁₉H₁₄N₄O₄ [M+H]⁺363.1088, measured value: 363.1084.

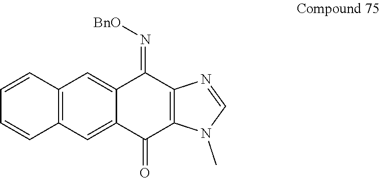 [SMILES: Cn1cnc2c1C(=O)c1cc3ccccc3cc1/C2=N\OCc1ccccc1]

(E) and (Z)-11-(benzyloxyimino)-3-methyl-3,11-dihydro-4H-anthraquinone[2,3-d]imidazole-4-one

¹H NMR (400 MHZ, DMSO-d₆) δ 9.67 (s, 1H), 8.79 (d, J=6.8 Hz, 2H), 8.73 (s, 1H), 8.27 (s, 1H), 8.23-8.08 (m, 6H), 8.00-7.95 (m, 1H), 7.73-7.55 (m, 11H), 7.46-7.33 (m, 8H), 5.57 (s, 2H), 5.53 (s, 3H), 4.06 (s, 5H), 4.01 (s, 3H).

HRMS (ESI): m/z theoretical value: C₁₉H₁₄N₄O₄ [M+H]⁺368.1394, measured value: 368.1397.

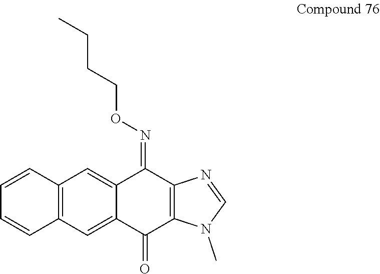 [SMILES: CCCCO/N=C1\c2cc3ccccc3cc2C(=O)c2c1ncn2C]

(E) and (Z)-11-(butoxyimino)-3-methyl-3,11-dihydro-4H-anthraquinone[2,3-d]imidazole-4-one

¹H NMR (400 MHZ, DMSO-d₆) δ 9.58 (s, 1H), 8.75 (d, J=2.1 Hz, 2H), 8.69 (s, 1H), 8.24 (s, 1H), 8.21-8.09 (m, 5H), 8.02-7.98 (m, 1H), 7.73-7.59 (m, 5H), 4.45 (dt, J=20.6, 6.6 Hz, 5H), 4.05 (s, 3H), 4.01 (s, 4H), 1.90-1.75 (m, 5H), 1.57-1.45 (m, 5H), 1.05-0.96 (m, 7H).

HRMS (ESI): m/z theoretical value: C₁₉H₁₄N₄O₄ [M+H]⁺334.2210, measured value: 334.2214.

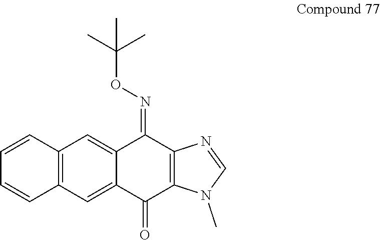 [SMILES: Cn1cnc2c1C(=O)c1cc3ccccc3cc1/C2=N\OC(C)(C)C]

(E) and (Z)-11-(tert-butoxyimino)-3-methyl-3,11-dihydro-4H-anthraquinone[2,3-d]imidazole-4-one

¹H NMR (400 MHZ, DMSO-d₆) δ 9.70 (s, 1H), 8.87 (s, 1H), 8.81 (s, 1H), 8.74 (s, 1H), 8.28-8.13 (m, 5H), 8.07-8.02 (m, 1H), 7.75-7.60 (m, 4H), 4.08 (s, 3H), 4.04 (s, 3H), 1.55 (s, 9H), 1.52 (s, 9H).

HRMS (ESI): m/z theoretical value: C₁₉H₁₄N₄O₄ [M+H]⁺334.2210, measured value: 334.2213.

### Example 7

2,3-Diamine-1,4-benzoquinone (20 g) was dissolved into 100 mL of formic acid, subjected to reflux reaction for 6 h, and cooled to room temperature, then poured into an ice-water mixture with the addition of ammonium hydroxide to regulate pH to 9-10, and the obtained solution was subjected to suction filtration and vacuum dried to obtain yellow solid 1,4-naphthoquinone imidazole (22 g). 1,4-naphthoquinone imidazole (0.3 g) and potassium hydroxide (0.15 g) were added to dimethyl sulfoxide solution (3 mL); the mixture was stirred at 50° C. for 30 min and added with iodomethane (0.2 mL), after reaction for 2, water was added to separate out solid; the solid was subjected to suction filtration, washed with water, and vacuum dried to obtain yellow solid 2-methyl-1,4-naphthoquinone imidazole (0.31 g). 2-methylnaphthoquinone imidazole (0.2 g), hydroxylamine hydrochloride (0.3 g), and pyridine (0.7 mL) were dissolved into ethanol (70 mL) and reacted for 36 h at 105° C.; at the end of reaction, solid was separated out from solution, directly subjected to suction filtration, and vacuum dried to obtain light yellow solid (E)-9-(hydroxylimino)-3-methyl-3,6,7,9-tetrahydro-4H-naphthol[2,3-d]imidazole-4-one (0.15 g). 106 mg of oximate product was added to 2 mL of a dimethyl sulfoxide solution; 56 μL of bromoethyl methyl ether and 104 mg of potassium hydroxide were added successively, and these materials were reacted for 2 h at room temperature. At the end of the reaction, water was added for quenching and dichloromethane was used for extraction; organic phase was washed with a saturated salt solution, dried with anhydrous magnesium sulfate, and concentrated, then isolated by silica gel column chromatography.

The oximated product was reacted with halohydrocarbons having different substitutes to prepare Compounds 78-82 according to the method in Example 7; other materials and reaction conditions for the compounds were kept the same and raw materials could be purchased commercially:

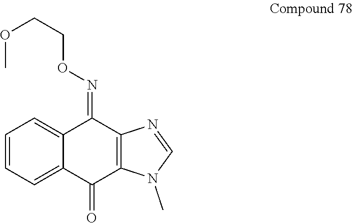 [SMILES: COCCO/N=C1\c2ccccc2C(=O)c2c1ncn2C]

(E)-9-((2-methoxyethoxy)imino)-3-methyl-3,9-dihydro-4H-naphthol[2,3-d]imidazole-4-one

¹H NMR (400 MHZ, DMSO-d₆) δ 8.34-8.24 (m, 2H), 8.16-8.09 (m, 1H), 7.76-7.70 (m, 1H), 7.65 (td, J=8.0, 1.2 Hz, 1H), 4.53 (t, J=4.0 Hz, 2H), 4.04 (s, 3H), 3.76 (t, J=4.0 Hz, 2H), 3.32 (s, 3H).

¹³C NMR (101 MHz, DMSO) δ 175.46, 145.74, 140.04, 139.32, 133.40, 133.14, 131.13, 129.95, 128.15, 126.17, 124.43, 76.07, 70.75, 58.76, 34.18.

HRMS (ESI): m/z theoretical value: C₁₅H₁₅N₃O₃ [M+H]⁺286.1186, measured value: 286.1188.

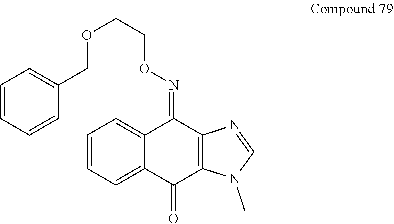 [SMILES: Cn1cnc2c1C(=O)c1ccccc1/C2=N\OCCOCc1ccccc1]

(E) and (Z)-9-((2-(benzyloxy) ethoxy)imino)-3-methyl-3,9-dihydro-4H-naphthol[2,3-d]imidazole-4-one

¹H NMR (400 MHZ, DMSO-d₆) δ 8.30-8.25 (m, 2H), 8.15-8.10 (m, 1H), 7.75-7.61 (m, 2H), 7.38-7.31 (m, 2H), 7.30-7.21 (m, 3H), 4.63-4.54 (m, 4H), 4.04 (s, 3H), 3.89-3.82 (m, 2H).

¹³C NMR (101 MHZ, DMSO-d₆) δ 175.45, 145.72, 140.12, 139.36, 138.95, 133.39, 133.08, 131.13, 129.92, 128.60, 128.15, 127.86, 127.74, 126.14, 124.43, 76.28, 72.40, 68.47, 34.18.

HRMS (ESI): m/z theoretical value: C₂₁H₁₉N₃O₃ [M+H]⁺362.1499, measured value: 362.1495.

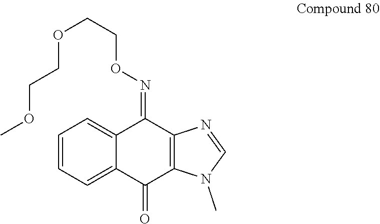 [SMILES: COCCOCCO/N=C1\c2ccccc2C(=O)c2c1ncn2C]

(E)-9-((2-(2-methoxyethoxy) ethoxy)imino)-3-methyl-3,9-dihydro-4H-naphthol[2,3-d]imidazole-4-one

¹H NMR (400 MHZ, DMSO-d₆) δ 8.31 (dd, J=8.0, 1.2 Hz, 1H), 8.24 (s, 1H), 8.13 (dd, J=8.0, 1.6 Hz, 1H), 7.72 (td, J=8.0, 1.6 Hz, 1H), 7.65 (td, J=8.0, 1.4 Hz, 1H), 4.55-4.49 (m, 2H), 4.04 (s, 3H), 3.86-3.80 (m, 2H), 3.64-3.59 (m, 2H), 3.46-3.42 (m, 2H), 3.22 (s, 3H).

¹³C NMR (101 MHz, DMSO-d₆) δ 175.45, 145.72, 140.07, 139.31, 133.40, 133.13, 131.13, 129.95, 128.15, 126.16, 124.45, 76.25, 71.78, 70.26, 69.26, 58.52, 34.18.

HRMS (ESI): m/z theoretical value: C₁₇H₁₉N₃O₄ [M+H]⁺330.1448, measured value: 330.1447.

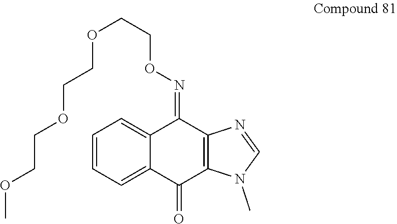 [SMILES: COCCOCCOCCO/N=C1\c2ccccc2C(=O)c2c1ncn2C]

(E) and (Z)-9-((2-(2-(2-methoxyethoxy) ethoxy) ethoxy)imino)-3-methyl-3,9-dihydro-4H-naphthol[2,3-d]imidazole-4-one

¹H NMR (400 MHZ, DMSO-d₆) δ 8.30 (dd, J=8.0, 1.2 Hz, 1H), 8.23 (s, 1H), 8.14-8.10 (m, 1H), 7.74-7.69 (m, 1H), 7.65 (td, J=8.0, 1.2 Hz, 1H), 4.55-4.51 (m, 2H), 4.04 (s, 3H), 3.87-3.82 (m, 2H), 3.65-3.59 (m, 2H), 3.54-3.47 (m, 4H), 3.39-3.35 (m, 2H), 3.19 (s, 3H).

¹³C NMR (101 MHz, DMSO-d₆) δ 175.44, 145.70, 140.04, 139.29, 133.39, 133.11, 131.10, 129.93, 128.12, 126.14, 124.43, 76.23, 71.70, 70.48, 70.24, 70.03, 69.26, 58.46, 34.16.

HRMS (ESI): m/z theoretical value: C₁₉H₂₃N₃O₅ [M+H]⁺374.1710, measured value: 374.1709.

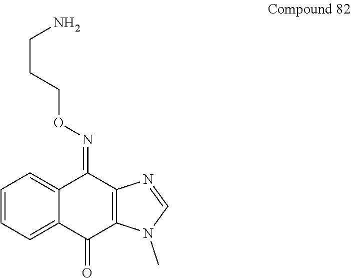 [SMILES: Cn1cnc2c1C(=O)c1ccccc1/C2=N\OCCCN]

(E)-9-((3-aminopropoxy)imino)-3-methyl-3,9-dihydro-4H-naphthol[2,3-d]imidazole-4-one

¹H NMR (400 MHZ, DMSO-d₆) δ 8.30-8.25 (m, 2H), 8.15-8.10 (m, 1H), 7.75-7.61 (m, 2H), 4.04 (s, 3H), 3.51 (t, J=8.0 Hz, 2H), 2.68 (t, J=8.0 Hz, 2H), 1.79-1.70 (m, 2H).

HRMS (ESI): m/z theoretical value: C₁₉H₂₃N₃O₅ [M+H]⁺285.1346, measured value: 285.1347.

### Example 8

2,3-Diamine-1,4-benzoquinone (20 g) was dissolved into 100 mL of formic acid, subjected to reflux reaction for 6 h, and cooled to room temperature, then poured into an ice-water mixture with the addition of ammonium hydroxide to regulate pH to 9-10, and the obtained solution was subjected to suction filtration and vacuum dried to obtain yellow solid 1,4-naphthoquinone imidazole (22 g). 1,4-naphthoquinone imidazole (0.3 g) and potassium hydroxide (0.15 g) were added to dimethyl sulfoxide solution (3 mL), respectively; the mixture was stirred at 50° C. for 30 min and added with iodobutane (0.18 mL), after reaction for 5 h, water was added to separate out solid; the solid was subjected to suction filtration, washed with water, and vacuum dried to obtain yellow solid 2-butyl-1,4-naphthoquinone imidazole (0.31 g). 2-butylnaphthoquinone imidazole (0.21 g), methoxyamine hydrochloride (0.3 g), and pyridine (0.7 mL) were dissolved into ethanol (70 mL) and reacted for 36 h at 105° C.; at the end of reaction, the obtained solution was concentrated and isolated by silica gel column chromatography to obtain light yellow product (0.18 g).

2,3-Diamine-1,4-naphthoquinone (20 g) was dissolved into 100 mL of formic acid and subjected to reflux reaction for 6 h to obtain anthraquinone imidazole; halohydrocarbons having different substitutes or aminoxyhydrochlorides having different substitutes were used to prepare Compounds 83-86 according to the method in Example 8; other materials and reaction conditions for the compounds were kept the same and raw materials could be purchased commercially:

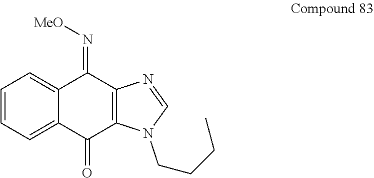 [SMILES: CCCCn1cnc2c1C(=O)c1ccccc1/C2=N\OC]

(E) and (Z)-3-butyl-9-(methoxyimino)-3,9-dihydro-4H-naphthol[2,3-d]imidazole-4-one

¹H NMR (400 MHZ, DMSO-d₆) δ 9.00 (dd, J=8.0, 1.2 Hz, 1H), 8.34-8.21 (m, 4H), 8.13 (dd, J=8.0, 1.4 Hz, 1H), 7.82-7.62 (m, 4H), 4.46 (t, J=7.2 Hz, 2H), 4.40 (t, J=7.0 Hz, 2H), 4.24 (s, 3H), 4.21 (s, 3H), 1.84-1.72 (m, 4H), 1.37-1.18 (m, 4H), 0.91 (t, J=8.0 Hz, 6H).

¹³C NMR (101 MHZ, DMSO) δ 175.14, 174.26, 146.33, 145.50, 145.23, 140.43, 140.02, 139.65, 133.69, 133.17, 133.10, 132.43, 131.59, 131.20, 131.15, 129.93, 127.94, 127.51, 127.14, 126.25, 125.85, 124.31, 64.75, 64.49, 46.53, 46.39, 32.75, 19.52, 19.50, 13.88.

HRMS (ESI): m/z theoretical value: C₁₆H₁₇N₃O₂ [M+H]⁺284.1394, measured value: 284.1394.

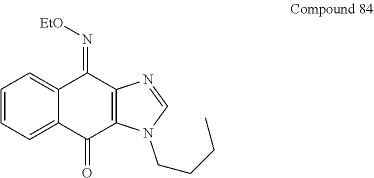 [SMILES: CCCCn1cnc2c1C(=O)c1ccccc1/C2=N\OCC]

(E) and (Z)-3-butyl-9-(ethoxyimino)-3,9-dihydro-4H-naphthol[2,3-d]imidazole-4-one

¹H NMR (400 MHZ, DMSO-d₆) δ 9.06 (d, J=8.0 Hz, 1H), 8.37-8.19 (m, 6H), 8.14 (d, J=8.0 Hz, 2H), 7.83-7.61 (m, 6H), 4.55-4.36 (m, 11H), 1.82-1.72 (m, 6H), 1.46-1.38 (m, 9H), 1.35-1.20 (m, 7H), 0.91 (t, J=8.0 Hz, 9H).

¹³C NMR (101 MHZ, DMSO) δ 175.16, 174.27, 146.52, 145.50, 145.27, 140.23, 139.77, 133.69, 133.43, 133.07, 132.42, 131.50, 131.13, 129.82, 128.00, 127.43, 127.13, 126.21, 125.79, 124.31, 72.74, 72.25, 46.52, 46.38, 32.78, 19.52, 19.50, 15.29, 15.06, 13.89.

HRMS (ESI): m/z theoretical value: C₁₇H₁₉N₃O₂ [M+H]⁺298.1550, measured value: 298.1549.

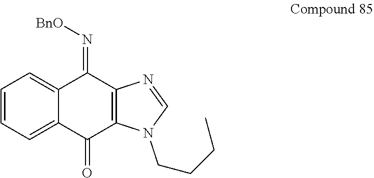 [SMILES: CCCCn1cnc2c1C(=O)c1ccccc1/C2=N\OCc1ccccc1]

(E) and (Z)-9-((benzyloxy)imino)-3-butyl-3,9-dihydro-4H-naphthol[2,3-d]imidazole-4-one

¹H NMR (400 MHZ, DMSO-d₆) δ 9.06 (dd, J=8.0, 1.2 Hz, 1H), 8.36-8.24 (m, 6H), 8.22 (s, 1H), 8.13 (dd, J=8.0, 1.6 Hz, 2H), 7.81-7.69 (m, 4H), 7.65 (dd, J=8.0, 1.4 Hz, 2H), 7.63-7.59 (m, 1H), 7.56-7.49 (m, 6H), 7.45-7.31 (m, 16H), 5.53 (s, 2H), 5.50 (s, 5H), 4.45 (t, J=7.2 Hz, 5H), 4.39 (t, J=7.1 Hz, 2H), 1.82-1.72 (m, 7H), 1.34-1.22 (m, 7H), 0.90 (td, J=8.0, 1.0 Hz, 11H).

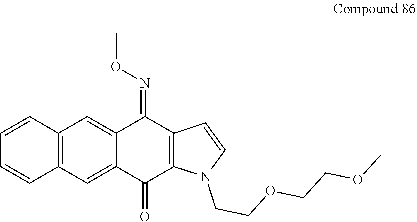 [SMILES: COCCOCCn1ccc2c1C(=O)c1cc3ccccc3cc1/C2=N\OC]

(E) and (Z)-11-((methoxy)imino)-3-(2-(2-methoxyethoxy)ethyl)-3,11-dihydro-4H-anthraquinone[2,3-d]imidazole-4-one

¹H NMR (400 MHZ, DMSO-d₆) δ 9.55 (s, 2H), 8.74 (d, J=9.3 Hz, 3H), 8.69 (s, 1H), 8.63 (s, 1H), 8.59 (s, 1H), 8.25-8.04 (m, 12H), 7.75-7.63 (m, 7H), 4.58 (dq, J=16.0, 5.2 Hz, 7H), 4.25 (s, 5H), 4.18 (s, 3H), 3.83-3.75 (m, 7H), 3.55-3.10 (m, 7H), 3.14-3.12 (m, 8H).

HRMS (ESI): m/z theoretical value: C₁₇H₁₉N₃O₂ [M+H]⁺380.1605, measured value: 380.1609.

### Example 9

2,3-Diamine-1,4-benzoquinone (20 g) was dissolved into 100 mL of acetic acid, subjected to reflux reaction for 6 h, and cooled to room temperature, then poured into an ice-water mixture with the addition of ammonium hydroxide to regulate pH to 9-10, and the obtained solution was subjected to suction filtration and vacuum dried to obtain yellow solid 1,4-naphthoquinone imidazole (22 g). 1,4-naphthoquinone imidazole (0.3 g) and potassium hydroxide (0.15 g) were added to dimethyl sulfoxide solution (3 mL), respectively; the mixture was stirred at 50° C. for 30 min and added with iodobutane (0.18 mL), after reaction for 5 h, water was added to separate out solid; the solid was subjected to suction filtration, washed with water, and vacuum dried to obtain yellow solid 2-butyl-1,4-naphthoquinone imidazole (0.31 g). 2-butylnaphthoquinone imidazole (0.21 g), methoxyamine hydrochloride (0.3 g), and pyridine (0.7 mL) were dissolved into ethanol (70 mL) and reacted for 36 h at 105° C.; at the end of reaction, the obtained solution was concentrated and isolated by silica gel column chromatography to obtain light yellow product (0.16 g).

2,3-Diamine-1,4-naphthoquinone (20 g) was dissolved into 100 mL of formic acid and subjected to reflux reaction for 6 h to obtain anthraquinone imidazole; or Compounds 87-88 may be prepared according to the method in Example 9; other materials and reaction conditions for the compounds were kept the same and raw materials could be purchased commercially:

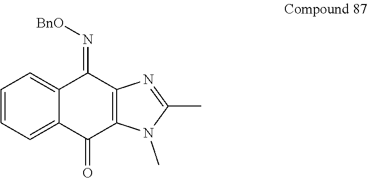 [SMILES: Cc1nc2c(n1C)C(=O)c1ccccc1/C2=N\OCc1ccccc1]

(E) and (Z)-9-((benzyloxy)imino)-2,3-dimethyl-3,9-dihydro-4H-naphthol[2,3-d]

¹H NMR (400 MHZ, DMSO-d₆) δ 8.99 (dd, J=8.0, 1.4 Hz, 1H), 8.22-8.16 (m, 4H), 8.05 (dd, J=7.8, 1.6 Hz, 3H), 7.73-7.68 (m, 1H), 7.66-7.62 (m, 3H), 7.58 (td, J=7.6, 1.4 Hz, 3H), 7.51-7.45 (m, 7H), 7.39-7.31 (m, 8H), 7.31-7.26 (m, 3H), 5.48 (s, 2H), 5.46 (s, 5H), 3.93 (s, 8H), 3.87 (s, 3H), 2.45 (s, 8H), 2.40 (s, 3H).

HRMS (ESI): m/z theoretical value: C₁₇H₁₉N₃O₂ [M+H]⁺332.1394, measured value: 332.1398.

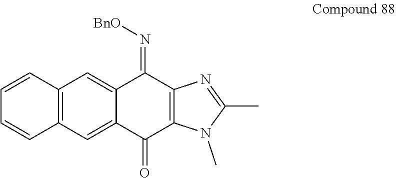 [SMILES: Cc1nc2c(n1C)C(=O)c1cc3ccccc3cc1/C2=N\OCc1ccccc1]

(E) and (Z)-11-((benzyloxy)imino)-2,3-dimethyl-3,11-dihydro-4H-anthraquinone[2,3-d]imidazole-4-one

¹H NMR (400 MHZ, DMSO-d₆) δ 8.69 (d, J=10.2 Hz, 2H), 8.13 (d, J=8.2 Hz, 1H), 8.05 (d, J=8.0 Hz, 1H), 7.69-7.52 (m, 2H), 7.39-7.31 (m, 3H), 5.59 (s, 2H), 4.03 (s, 3H), 2.72 (s, 3H).

HRMS (ESI): m/z theoretical value: C₁₇H₁₉N₃O₂ [M+H]⁺382.1550, measured value: 382.1558.

### Example 10

2,3-Diamine-1,4-benzoquinone (4 g) was dissolved into 13 mL of dimethyl sulfoxide, added with 3,4,5-trimethoxybenzaldehyde (2.5 mL), and subjected to open-ended reaction for 13 h at 80° C., and cooled to room temperature, then slowly added with water, solid was separated out, subjected to suction filtration and vacuum dried to obtain yellow solid 2-(3,4,5-trimethoxyphenyl)-1,4-naphthoquinone imidazole (0.15 g). Naphthoquinone imidazole (0.4 g) and potassium hydroxide (0.15 g) were added to dimethyl sulfoxide solution (3 mL), respectively; the mixture was stirred at 50° C. for 30 min and added with iodobutane (0.17 mL), after reaction for 5 h, water was added to separate out solid; the solid was subjected to suction filtration, washed with water, and vacuum dried to obtain yellow solid 1-butyl-2-(3,4,5-trimethoxyphenyl)-1,4-naphthoquinone imidazole (0.35 g). Naphthoquinone imidazole (0.42 g), benzyloxyamine hydrochloride (0.64 g), and pyridine (0.7 mL) were dissolved into ethanol (70 mL) and reacted for 36 h at 105° C.; at the end of reaction, the obtained solution was concentrated and isolated by silica gel column chromatography to obtain light yellow product (0.36 g).

2,3-Diamine-1,4-naphthoquinone (4 g) was dissolved into 13 mL of dimethyl sulfoxide and reacted with different aldehydes; or aminoxyhydrochlorides having different substitutes were used to prepare Compounds 89-90 according to the method in Example 10; other materials and reaction conditions for the compounds were kept the same and raw materials could be purchased commercially:

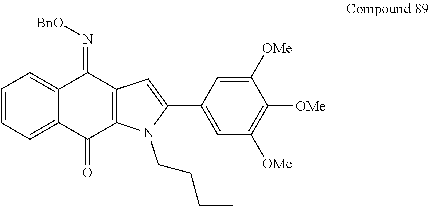 [SMILES: CCCCn1c(-c2cc(OC)c(OC)c(OC)c2)cc2c1C(=O)c1ccccc1/C2=N\OCc1ccccc1]

(E) and (Z)-9-((benzyloxy)imino)-3-butyl-2-(3,4,5-trimethoxyphenyl)-3,9-dihydro-4H-naphthol[2,3-d]imidazole-4-one

¹H NMR (400 MHZ, DMSO-d₆) δ 9.04 (d, J=8.0 Hz, 1H), 8.27 (t, J=7.0 Hz, 2H), 8.13 (dd, J=7.8, 1.6 Hz, 2H), 7.72 (dt, J=15.2, 7.4 Hz, 3H), 7.63 (t, J=7.6 Hz, 2H), 7.57-7.50 (m, 3H), 7.47 (d, J=7.4 Hz, 2H), 7.40-7.18 (m, 14H), 6.98 (s, 3H), 6.95 (s, 2H), 5.52 (s, 2H), 5.48 (s, 3H), 4.53 (s, 3H), 4.47 (t, J=7.4 Hz, 3H), 4.41 (t, J=7.6 Hz, 2H), 3.82 (s, 4H), 3.80 (s, 7H), 3.72 (d, J=1.2 Hz, 5H), 1.70-1.65 (m, 5H), 1.21-1.15 (m, 5H), 0.85-0.70 (m, 7H).

HRMS (ESI): m/z theoretical value: C₁₇H₁₉N₃O₂ [M+H]⁺526.2337, measured value: 526.2337.

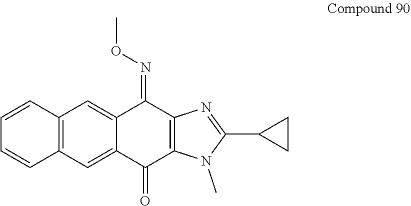 [SMILES: CO/N=C1\c2cc3ccccc3cc2C(=O)c2c1nc(C1CC1)n2C]

(E) and (Z)-2-cyclopropyl-11-(methoxyimino)-3-methyl-3,11-dihydro-4H-anthraquinone[2,3-d]imidazole-4-one

¹H NMR (400 MHZ, DMSO-d₆) δ 8.55 (s, 2H), 8.18-8.12 (m, 2H), 7.73-7.60 (m, 2H), 2.15-2.01 (m, 1H), 1.12-0.97 (m, 4H), 4.05 (s, 3H), 3.84 (s, 3H).

HRMS (ESI): m/z theoretical value: C₁₇H₁₉N₃O₂ [M+H]⁺332.1394, measured value: 332.1395.

### Example 11

Compound 1 (0.05 mmol) and 31 μL of iodomethane were added to 3 mL acetonitrile for reaction for 24 h at 120° C. At the end of the reaction, solvent was removed to obtain a yellow solid, and the solid was pulped for half an hour and subjected to suction filtration to obtain the final product.

Different synthetic intermediate compounds above were used to prepare Compounds 91-118 according to the method in Example 11; other materials and reaction conditions for the compounds were kept the same and raw materials could be purchased commercially:

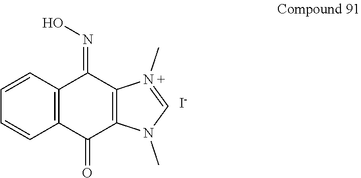

(E)-1-methyl-4-(hydroxylimino)-3-methyl-9-oxo-4,9-dihydro-1H-naphthol[2,3-d]imidazole-3-iodide

¹H NMR (500 MHZ, DMSO-d₆) δ 14.50 (s, 1H), 9.57 (s, 1H), 9.25 (dd, J=10.0, 0.5 Hz, 1H), 8.33 (dd, J=10.0, 1.0 Hz, 1H), 8.01-7.93 (m, 1H), 7.85 (td, J=5.0, 1.5 Hz, 1H), 4.19 (s, 3H), 4.10 (s, 3H).

¹³C NMR (126 MHz, DMSO-d₆) δ 174.28, 143.85, 138.38, 135.34, 135.31, 131.90, 131.49, 130.83, 127.34, 127.16, 125.39, 38.98, 36.41.

HRMS (ESI-TOF) m/z theoretical value: C₁₃H₁₂N₃O₂ [M-I]⁺, 242.0924, measured value: 242.0928.

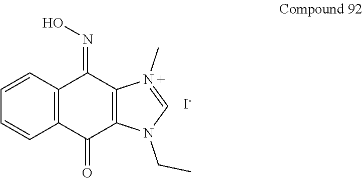

(E)-1-ethyl-4-(hydroxylimino)-3-methyl-9-oxo-4,9-dihydro-1H-naphthol[2,3-d]imidazole-3-iodide

¹H NMR (500 MHZ, DMSO-d₆) δ 14.49 (s, 1H), 9.67 (s, 1H), 9.26 (dd, J=10.0, 1.0 Hz, 1H), 8.34 (dd, J=10.0, 1.0 Hz, 1H), 7.96 (td, J=10.0, 1.5 Hz, 1H), 7.85 (td, J=10.0, 1.0 Hz, 1H), 4.63 (q, J=7.5 Hz, 2H), 4.11 (s, 3H), 1.53 (t, J=5.0 Hz, 3H).

¹³C NMR (126 MHz, DMSO-d₆) δ 174.01, 143.12, 138.52, 135.86, 135.28, 131.83, 131.37, 130.85, 127.39, 127.03, 124.77, 44.93, 39.09, 15.42.

HRMS (ESI-TOF) m/z theoretical value: C₁₄H₁₄N₃O₂ [M-I]⁺, 256.1081, measured value: 256.1078.

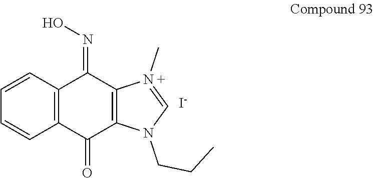

(E)-4-(hydroxylimino)-3-methyl-9-oxo-1-propyl-4,9-dihydro-1H-naphthol[2,3-d]imidazole-3-iodide

¹H NMR (500 MHZ, DMSO-d₆) δ 14.51 (s, 1H), 9.67 (s, 1H), 9.25 (dd, J=10.0, 1.0 Hz, 1H), 8.34 (dd, J=10.0, 1.5 Hz, 1H), 8.00-7.93 (m, 1H), 7.85 (td, J=10.0, 1.0 Hz, 1H), 4.57 (t, J=7.0 Hz, 2H), 4.11 (s, 3H), 1.96-1.85 (m, 2H), 0.96 (t, J=7.5 Hz, 3H).

¹³C NMR (126 MHz, DMSO-d₆) & 174.09, 143.40, 138.51, 135.95, 135.29, 131.88, 131.43, 130.91, 127.42, 127.08, 124.84, 50.68, 39.17, 23.13, 10.92.

HRMS (ESI-TOF) m/z theoretical value: C₁₅H₁₆N₃O₂ [M-I]⁺, 270.1237, measured value: 270.1244.

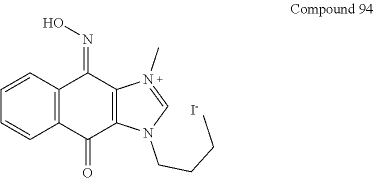

(E)-1-butyl-4-(hydroxylimino)-3-methyl-9-oxo-4,9-dihydro-1H-naphthol[2,3-d]imidazole-3-iodide

¹H NMR (500 MHZ, DMSO-d₆) δ 14.50 (s, 1H), 9.67 (s, 1H), 9.25 (dd, J=10.0, 0.5 Hz, 1H), 8.34 (dd, J=10.0, 1.5 Hz, 1H), 7.96 (td, J=10.0, 1.5 Hz, 1H), 7.85 (td, J=10.0, 1.5 Hz, 1H), 4.60 (t, J=7.0 Hz, 2H), 4.10 (s, 3H), 1.91-1.85 (m, 2H), 1.44-1.33 (m, 2H), 0.95 (t, J=7.5 Hz, 3H).

¹³C NMR (126 MHz, DMSO-d₆) δ 174.06, 143.37, 138.52, 135.93, 135.28, 131.85, 131.40, 130.89, 127.42, 127.06, 124.80, 49.00, 39.16, 31.69, 19.28, 13.86.

HRMS (ESI-TOF) m/z theoretical value: C₁₆H₁₈N₃O₂ [M-I]⁺, 284.1394, measured value: 284.1393.:

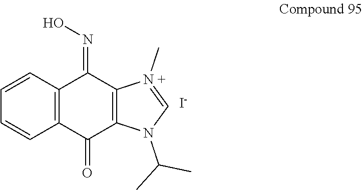

(E)-4-(hydroxylimino)-1-isopropyl-3-methyl-9-oxo-4,9-dihydro-1H-naphthol[2,3-d]imidazole-3-iodide

¹H NMR (400 MHZ, DMSO-d₆) δ 14.50 (s, 1H), 9.81 (s, 1H), 9.24 (dd, J=8.0, 1.2 Hz, 1H), 8.35 (dd, J=8.0, 1.6 Hz, 1H), 8.04-7.90 (m, 1H), 7.85 (td, J=8.0, 1.2 Hz, 1H), 5.49-5.40 (m, 1H), 4.11 (s, 3H), 1.61 (d, J=8.0 Hz, 6H).

¹³C NMR (101 MHz, DMSO-d₆) δ 174.04, 141.76, 138.52, 135.96, 135.23, 131.88, 131.36, 131.04, 127.51, 126.85, 124.69, 52.90, 39.23, 22.54.

HRMS (ESI-TOF) m/z theoretical value: C₁₅H₁₆N₃O₂ [M-I]⁺, 270.12374, measured value: 270.1237.:

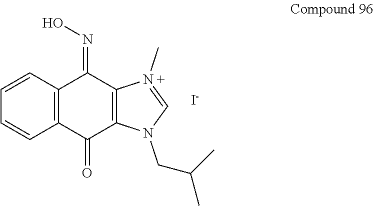

(E)-4-(hydroxylimino)-1-isobutyl-3-methyl-9-oxo-4,9-dihydro-1H-naphthol[2,3-d]imidazole-3-iodide

¹H NMR (500 MHZ, DMSO-d₆) δ 14.51 (s, 1H), 9.68 (s, 1H), 9.25 (dd, J=10.0, 0.5 Hz, 1H), 8.33 (dd, J=10.0, 1.0 Hz, 1H), 7.99-7.93 (m, 1H), 7.85 (td, J=10.0, 1.0 Hz, 1H), 4.44 (d, J=10.0 Hz, 2H), 4.12 (s, 3H), 2.27-2.10 (m, 1H), 0.98 (d, J=5.0 Hz, 6H).

¹³C NMR (126 MHz, DMSO-d₆) δ 174.14, 143.52, 138.52, 136.06, 135.31, 131.87, 131.41, 130.92, 127.42, 127.08, 124.84, 55.59, 39.26, 29.14, 19.66.

HRMS (ESI-TOF) m/z theoretical value: C₁₆H₁₈N₃O₂ [M-I]⁺, 284.1394, measured value: 284.1387.:

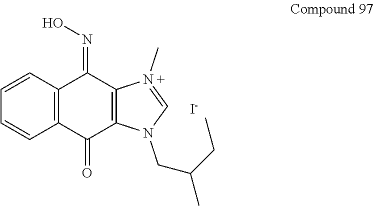

(E)-4-(hydroxylimino)-3-methyl-1-(2-methylbutyl)-9-oxo-4,9-dihydro-1H-naphthol[2,3-d]imidazole-3-iodide

¹H NMR (400 MHZ, DMSO-d₆) δ 14.50 (s, 1H), 9.69 (s, 1H), 9.25 (dd, J=8.0, 0.8 Hz, 1H), 8.33 (dd, J=8.0, 1.4 Hz, 1H), 8.01-7.91 (m, 1H), 7.84 (td, J=8.0, 1.2 Hz, 1H), 4.56 (dd, J=12.0, 4.0 Hz, 1H), 4.42-4.35 (m, 1H), 4.12 (s, 3H), 2.07-1.90 (m, 1H), 1.53-1.40 (m, 1H), 1.33-1.19 (m, 1H), 1.00-0.87 (m, 6H).

¹³C NMR (101 MHz, DMSO-d₆) δ 174.12, 143.58, 138.52, 136.06, 135.29, 131.86, 131.40, 130.91, 127.43, 127.07, 124.87, 54.38, 39.27, 35.19, 26.25, 16.46, 11.30.

HRMS (ESI-TOF) m/z theoretical value: C₁₇H₂₀N₃O₂ [M-I]⁺, 298.1550, measured value: 298.1551.

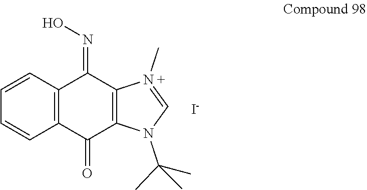

(E)-1-(tert-butyl)-4-(hydroxylimino)-3-methyl-9-oxo-4,9-dihydro-1H-naphthol[2,3-d]imidazole-3-iodide

¹H NMR (400 MHZ, DMSO-d₆) δ 14.50 (s, 1H), 9.67 (s, 1H), 9.19 (dd, J=8.0, 1.2 Hz, 1H), 8.38 (dd, J=8.0, 1.4 Hz, 1H), 7.98-7.91 (m, 1H), 7.89-7.79 (m, 1H), 4.11 (s, 3H), 1.81 (s, 9H).

¹³C NMR (101 MHz, DMSO-d₆) δ 173.08, 142.92, 138.84, 138.42, 134.98, 131.90, 131.45, 130.93, 1210.06, 126.24, 126.02, 63.89, 36.40, 28.72.

HRMS (ESI-TOF) m/z theoretical value: C₁₆H₁₈N₃O₂ [M-I]⁺, 284.1394, measured value: 284.1395.

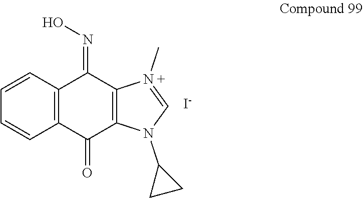

(E)-1-cyclopropyl-4-(hydroxylimino)-3-methyl-9-oxo-4,9-dihydro-1H-naphthol[2,3-d]imidazole-3-iodide

¹H NMR (500 MHz, DMSO-d₆) δ 14.48 (s, 1H), 9.73 (s, 1H), 9.31-9.16 (m, 1H), 8.35 (dd, J=10.0, 1.5 Hz, 1H), 7.96 (td, J=10.0, 1.0 Hz, 1H), 7.86 (td, J=10.0, 1.0 Hz, 1H), 4.16-4.05 (m, 1H), 4.06 (s, 3H), 1.31-1.18 (m, 4H).

¹³C NMR (126 MHz, DMSO-d₆) δ 173.42, 143.46, 138.43, 135.56, 135.19, 131.93, 131.43, 131.00, 127.40, 126.93, 126.42, 39.02, 31.83, 7.48.

HRMS (ESI-TOF) m/z theoretical value: C₁₅H₁₄N₃O₂ [M-I]⁺, 268.1081, measured value: 268.1081.

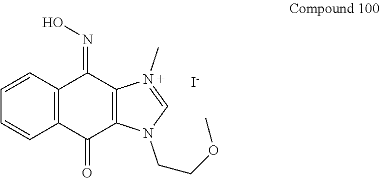

(E)-4-(hydroxylimino)-1-(2-methoxyethyl)-3-methyl-9-oxo-4,9-dihydro-1H-naphthol[2,3-d]imidazole-3-iodide

¹H NMR (500 MHZ, DMSO-d₆) δ 14.51 (s, 1H), 9.65 (s, 1H), 9.25 (d, J=10.0, 1H), 8.33 (dd, J=10.0, 1.5 Hz, 1H), 7.97 (td, J=10.0, 1.5 Hz, 1H), 7.85 (td, J=10.0, 1.0 Hz, 1H), 4.81 (t, J=5.0 Hz, 2H), 4.14 (s, 3H), 3.81 (t, J=5.0 Hz, 2H), 3.30 (s, 3H).

¹³C NMR (126 MHZ, DMSO-d₆) δ 174.24, 143.67, 138.44, 135.78, 135.33, 131.86, 131.44, 130.88, 127.41, 127.09, 124.77, 69.37, 58.66, 48.91, 39.15.

HRMS (ESI-TOF) m/z theoretical value: C₁₅H₁₆N₃O₃ [M-I]⁺, 286.1186, measured value: 286.1187.:

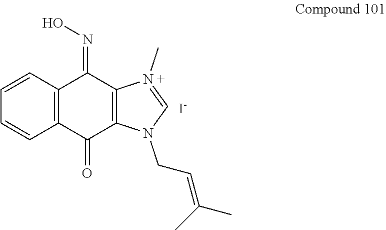

(E)-4-(hydroxylimino)-3-methyl-1-(3-methyl-2-ene-1-yl)-9-oxo-4,9-dihydro-1H-naphthol[2,3-d]imidazole-3-iodide

¹H NMR (500 MHZ, DMSO-d₆) δ 14.50 (s, 1H), 9.62 (s, 1H), 9.25 (dd, J=10.0, 1.0 Hz, 1H), 8.34 (dd, J=10.0, 1.5 Hz, 1H), 7.96 (td, J=10.0, 1.5 Hz, 1H), 7.85 (td, J=10.0, 1.0 Hz, 1H), 5.55-5.45 (m, 1H), 5.22 (d, J=5.0 Hz, 2H), 4.11 (s, 3H), 1.84 (s, 3H), 1.80 (s, 3H).

¹³C NMR (126 MHz, DMSO-d₆) δ 174.14, 142.89, 141.39, 138.44, 135.86, 135.31, 131.89, 131.45, 130.86, 127.42, 127.07, 124.81, 116.94, 47.08, 39.11, 26.02, 18.66.

HRMS (ESI-TOF) m/z theoretical value: C₁₇H₁₈N₃O₂ [M-I]⁺, 296.1394, measured value: 296.1390.

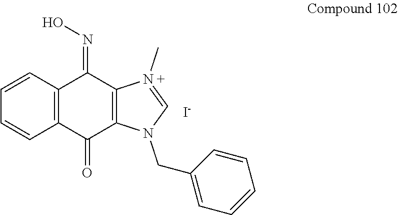

(E)-1-benzyl-4-(hydroxylimino)-3-methyl-9-oxo-4,9-dihydro-1H-naphthol[2,3-d]imidazole-3-iodide

¹H NMR (500 MHZ, DMSO-d₆) δ 14.53 (s, 1H), 9.76 (s, 1H), 9.24 (d, J=10.0 Hz, 1H), 8.30 (dd, J=10.0, 1.5 Hz, 1H), 7.95 (td, J=10.0, 1.5 Hz, 1H), 7.83 (td, J=10.0, 1.0 Hz, 1H), 7.51-7.45 (m, 2H), 7.45-7.36 (m, 3H), 5.88 (s, 2H), 4.13 (s, 3H).

¹³C NMR (126 MHz, DMSO-d₆) δ 174.00, 143.75, 138.46, 136.14, 135.37, 134.74, 131.87, 131.44, 130.76, 129.34, 129.11, 128.56, 127.39, 127.12, 124.57, 51.80, 39.35.

HRMS (ESI-TOF) m/z theoretical value: C₁₉H₁₆N₃O₂ [M-I]⁺, 318.1237, measured value: 318.1233.

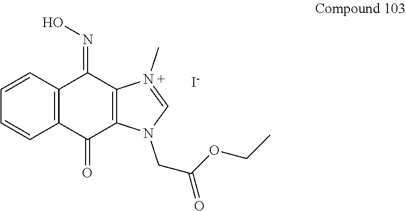

(E)-1-(2-ethoxy-2-ethoxy)-4-(hydroxylimino)-3-methyl-9-oxo-4,9-dihydro-1H-naphthol[2,3-d]imidazole-3-iodide

¹H NMR (500 MHz, DMSO-d₆) δ 14.62 (s, 1H), 9.61 (s, 1H), 9.27 (d, J=10.0 Hz, 1H), 8.30 (dd, J=10.0, 1.5 Hz, 1H), 7.99 (td, J=10.0, 1.5 Hz, 1H), 7.88-7.82 (m, 1H), 5.58 (s, 2H), 4.26 (q, J=7.0 Hz, 2H), 4.19 (s, 3H), 1.27 (t, J=7.0 Hz, 3H).

¹³C NMR (126 MHZ, DMSO-d₆) δ 174.04, 166.57, 144.24, 138.22, 135.63, 135.54, 131.97, 131.59, 130.42, 127.36, 127.23, 124.83, 62.54, 49.97, 39.38, 14.51.

HRMS (ESI-TOF) m/z theoretical value: C₁₆H₁₆N₃O₄ [M-I]⁺, 314.1135, measured value: 314.1138.:

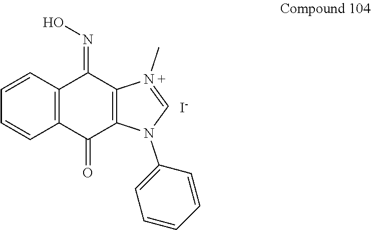

(E)-4-(hydroxylimino)-3-methyl-9-oxo-1-phenyl-4,9-dihydro-1H-naphthol[2,3-d]imidazole-3-iodide

¹H NMR (500 MHZ, DMSO-d₆) δ 14.60 (s, 1H), 10.00 (s, 1H), 9.28 (d, J=5.0, Hz, 1H), 8.23 (dd, J=10.0, 1.5 Hz, 1H), 8.01-7.95 (m, 1H), 7.84 (td, J=10.0, 1.0 Hz, 1H), 7.77-7.74 (m, 2H), 7.71-7.67 (m, 3H), 4.19 (s, 3H).

¹³C NMR (126 MHz, DMSO-d₆) δ 172.91, 144.41, 138.47, 135.61, 135.26, 133.99, 131.95, 131.41, 131.18, 131.04, 129.83, 127.44, 126.91, 126.62, 125.20, 39.40.

HRMS (ESI-TOF) m/z theoretical value: C₁₈H₁₄N₃O₂ [M-I]⁺, 304.1081, measured value: 304.1085.

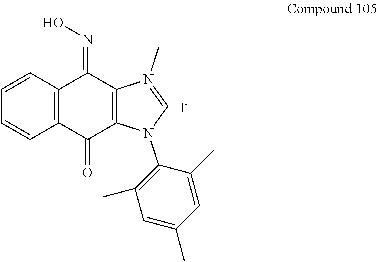

(E)-4-(hydroxylimino)-1-sym-trimethyl-3-methyl-9-oxo-4,9-dihydro-1H-naphthol[2,3-d]imidazole-3-iodide

¹H NMR (500 MHz, DMSO-d₆) δ 14.58 (s, 1H), 9.81 (s, 1H), 9.29 (dd, J=10.0, 0.5 Hz, 1H), 8.18 (dd, J=10.0, 1.0 Hz, 1H), 8.03-7.93 (m, 1H), 7.82 (td, J=10.0, 1.0 Hz, 1H), 7.16 (s, 2H), 4.20 (s, 3H), 2.38 (s, 3H), 2.04 (s, 6H).

¹³C NMR (126 MHz, DMSO-d₆) δ 173.08, 144.26, 140.75, 138.74, 136.36, 135.27, 135.12, 131.82, 131.44, 130.87, 130.41, 129.56, 127.35, 127.27, 124.97, 39.78, 21.15, 17.54.

HRMS (ESI-TOF) m/z theoretical value: C₂₁H₂₀N₃O₂ [M-I]⁺, 346.1550, measured value: 346.1555.

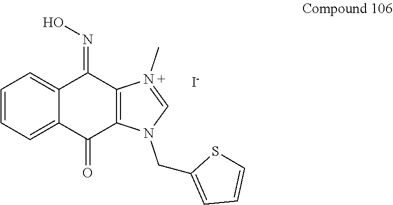

(E)-4-(hydroxylimino)-3-methyl-9-oxo-1-(thiophene-2-yl-methyl)-4,9-dihydro-1H-naphthol[2,3-d]imidazole-3-iodide

¹H NMR (400 MHz, DMSO-d₆) δ 13.01 (s, 1H), 8.49 (s, 1H), 8.27 (d, J=7.8 Hz, 1H), 8.12 (dd, J=8.0, 1.6 Hz, 1H), 7.69 (t, J=7.6 Hz, 1H), 7.60 (t, J=7.1 Hz, 1H), 7.43 (d, J=5.9 Hz, 1H), 7.19 (d, J=3.0 Hz, 1H), 6.95 (dd, J=5.1, 3.5 Hz, 1H), 5.88 (s, 2H), 4.28 (s, 3H).

HRMS (ESI-TOF) m/z theoretical value: C₁₇H₁₄N₃O₂S [M-I]⁺, 324.0801, measured value: 324.0802.

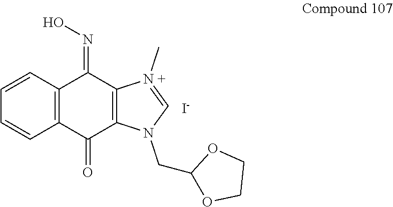

(E)-1-((1,3-dioxoheterocycle-2-yl)methyl)-4-(hydroxylimino)-3-methyl-9-oxo-4,9-dihydro-1H-naphthol[2,3-d]imidazole-3-iodide

¹H NMR (500 MHZ, DMSO-d₆) & 14.53 (s, 1H), 9.64 (s, 1H), 9.25 (dd, J=10.0, 0.5 Hz, 1H), 8.33 (dd, J=10.0, 1.5 Hz, 1H), 8.01-7.93 (m, 1H), 7.85 (td, J=10.0, 1.0 Hz, 1H), 5.34 (t, J=3.5 Hz, 1H), 4.91 (d, J=5.0 Hz, 2H), 4.16 (s, 3H), 3.86 (s, 4H).

¹³C NMR (126 MHz, DMSO-d₆) & 174.18, 144.11, 138.36, 135.52, 135.39, 131.89, 131.45, 130.83, 127.43, 127.06, 125.01, 99.99, 65.37, 49.50, 39.28.

HRMS (ESI-TOF) m/z theoretical value: C₁₆H₁₅N₃O₄ [M-I]⁺, 314.1135, measured value: 314.1136.

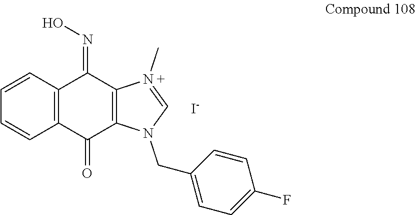

(E)-1-(4-fluorobenzyl)-4-(hydroxylimino)-3-methyl-9-oxo-4,9-dihydro-1H-naphthol[2,3-d]imidazole-3-iodide

¹H NMR (500 MHZ, DMSO-d₆) δ 14.54 (s, 1H), 9.71 (s, 1H), 9.24 (dd, J=10.0, 0.5 Hz, 1H), 8.31 (dd, J=10.0, 1.5 Hz, 1H), 7.96 (td, J=10.0, 1.5 Hz, 1H), 7.83 (td, J=10.0, 1.0 Hz, 1H), 7.57 (dd, J=10.0, 5.0 Hz, 2H), 7.28 (t, J=10.0 Hz, 2H), 5.85 (s, 2H), 4.12 (s, 3H).

¹³C NMR (126 MHz, DMSO-d₆) δ 174.02, 163.66 (161.71, J=243.8 Hz), 143.70, 138.45, 136.13, 135.39, 131.89, 131.45, 131.20 (131.14, J=7.5 Hz), 130.89 (130.87, J=2.5 Hz), 130.76, 127.40, 127.12, 124.57, 116.29 (116.12, J=21.3 Hz), 51.15, 39.33.

HRMS (ESI-TOF) m/z theoretical value: C₁₉H₁₅FN₃O₂ [M-I]⁺, 336.1143, measured value: 336.1138.

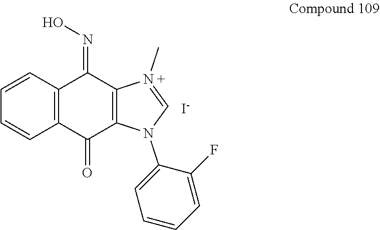

(E)-1-(2-fluorophenyl)-4-(hydroxylimino)-3-methyl-9-oxo-4,9-dihydro-1H-naphthol[2,3-d]imidazole-3-iodide

¹H NMR (500 MHZ, DMSO-d₆) δ 14.68 (s, 1H), 10.08 (s, 1H), 9.29 (dd, J=10.0, 1.0 Hz, 1H), 8.24 (dd, J=10.0, 1.5 Hz, 1H), 8.03-7.95 (m, 1H), 7.89-7.77 (m, 3H), 7.68-7.61 (m, 1H), 7.54 (td, J=10.0, 1.0 Hz, 1H), 4.21 (s, 3H).

¹³C NMR (126 MHZ, DMSO) δ 172.95, 157.58 (155.57, J=251.3 Hz), 145.03, 138.34, 135.74, 135.52, 133.83 (133.77, J=7.5 Hz), 132.01, 131.52, 130.65, 128.91, 127.34, 127.06, 125.88 (125.85, J=3.8 Hz), 125.17, 121.97 (121.87, J=12.5 Hz), 117.28 (117.13, J=18.8 Hz), 39.62.

HRMS (ESI-TOF) m/z theoretical value: C₁₈H₁₃FN₃O₂ [M-I]⁺, 322.0986, measured value: 322.0986.

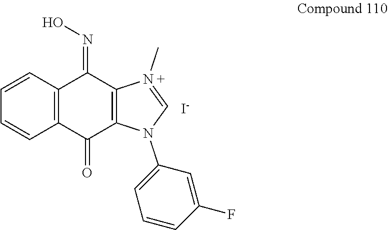

(E)-1-(3-fluorophenyl)-4-(hydroxylimino)-3-methyl-9-oxo-4,9-dihydro-1H-naphthol[2,3-d]imidazole-3-iodide

¹H NMR (400 MHZ, DMSO-d₆) δ 14.64 (s, 1H), 10.04 (d, J=0.4 Hz, 1H), 9.28 (dd, J=8.0, 0.8 Hz, 1H), 8.25 (dd, J=8.0, 1.2 Hz, 1H), 8.03-7.95 (m, 1H), 7.85 (td, J=8.0, 1.2 Hz, 1H), 7.80-7.71 (m, 2H), 7.66-7.54 (m, 2H), 4.20 (s, 3H).

¹³C NMR (101 MHz, DMSO) δ 172.90, 163.16 (160.72, J=244.0 Hz), 144.62, 138.38, 135.43, 135.33, 135.07 (134.96, J=11.0 Hz), 131.98, 131.76 (131.67, J=9.0 Hz), 131.41, 130.99, 127.49, 126.87, 125.21, 123.05 (123.02, J=3.0 Hz), 118.47 (118.26, J=21.0 Hz), 114.78 (114.53, J=25.0 Hz), 39.45.

HRMS (ESI-TOF) m/z theoretical value: C₁₈H₁₃FN₃O₂ [M-I]⁺, 322.0986, measured value: 322.0982.:

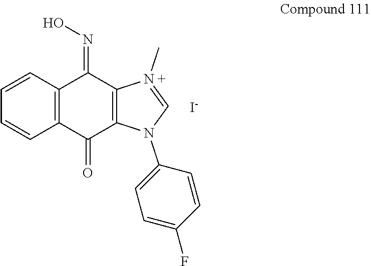

(E)-1-(4-fluorophenyl)-4-(hydroxylimino)-3-methyl-9-oxo-4,9-dihydro-1H-naphthol[2,3-d]imidazole-3-iodide

¹H NMR (400 MHZ, DMSO-d₆) δ 14.60 (s, 1H), 9.96 (s, 1H), 9.26 (d, J=8.0 Hz, 1H), 8.22 (d, J=8.0 Hz, 1H), 7.95 (t, J=8.0 Hz, 1H), 7.86-7.72 (m, 3H), 7.54 (t, J=8.0 Hz, 2H), 4.17 (s, 3H).

¹³C NMR (126 MHz, DMSO) δ 172.97, 164.38 (162.40, J=1910.0 Hz), 144.56, 138.40, 135.41 (135.31, J=10.0 Hz), 131.97, 131.44, 130.99, 130.26, 130.24, 129.22 (129.14, J=10.0 Hz), 127.44, 126.91, 125.32, 116.93 (116.74, J=19.0 Hz), 39.39.

HRMS (ESI-TOF) m/z theoretical value: C₁₈H₁₃FN₃O₂ [M-I]⁺, 322.0986, measured value: 322.0991.

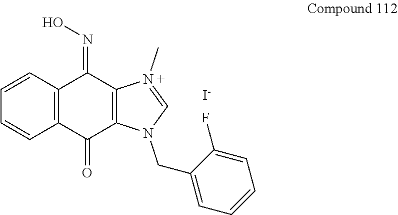

(E)-1-(2-fluorobenzyl)-4-(hydroxylimino)-3-methyl-9-oxo-4,9-dihydro-1H-naphthol[2,3-d]imidazole-3-iodide

¹H NMR (500 MHZ, DMSO-d₆) δ 14.55 (s, 1H), 9.71 (s, 1H), 9.25 (dd, J=10.0, 1.0 Hz, 1H), 8.29 (dd, J=10.0, 1.5 Hz, 1H), 7.96 (td, J=10.0, 1.5 Hz, 1H), 7.83 (td, J=10.0, 1.0 Hz, 1H), 7.52-7.43 (m, 2H), 7.37-7.30 (m, 1H), 7.24 (td, J=10.0, 1.0 Hz, 1H), 5.94 (s, 2H), 4.14 (s, 3H).

¹³C NMR (126 MHZ, DMSO-d₆) δ 173.93, 161.62 (159.66, J=245.0 Hz), 144.04, 138.44, 136.14, 135.42, 131.90, 131.61 (131.55, J=7.5 Hz), 131.46, 130.70, 130.67, 127.40, 127.13, 125.35 (125.32, J=3.8 Hz), 124.67, 121.80 (121.69, J=13.8 Hz), 116.23 (116.07, J=20.0 Hz), 46.44 (46.41, J=3.8 Hz), 39.32.

HRMS (ESI-TOF) m/z theoretical value: C₁₉H₁₅FN₃O₂ [M-I]⁺, 336.1143, measured value: 336.1145.

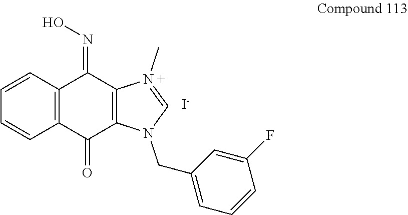

(E)-1-(3-fluorobenzyl)-4-(hydroxylimino)-3-methyl-9-oxo-4,9-dihydro-1H-naphthol[2,3-d]imidazole-3-iodide

¹H NMR (500 MHZ, DMSO-d₆) δ 14.55 (s, 1H), 9.74 (s, 1H), 9.25 (dd, J=10.0, 1.0 Hz, 1H), 8.30 (dd, J=10.0, 2.0 Hz, 1H), 7.96 (td, J=10.0, 1.5 Hz, 1H), 7.83 (td, J=10.0, 1.0 Hz, 1H), 7.50-7.45 (m, 1H), 7.36-7.29 (m, 2H), 7.26-7.18 (m, 1H), 5.89 (s, 2H), 4.13 (s, 3H).

¹³C NMR (126 MHz, DMSO-d₆) δ 173.96, 163.70 (161.76, J=242.5 Hz), 143.95, 138.48, 137.47 (137.41, J=7.5 Hz), 136.15, 135.39, 131.88, 131.45, 131.39 (131.32, J=8.8 Hz), 130.75, 127.40, 127.15, 124.61, 124.59, 115.98 (115.82, J=20.0 Hz), 115.41 (115.23, J=22.5 Hz), 51.21, 39.38.

HRMS (ESI-TOF) m/z theoretical value: C₁₉H₁₅FN₃O₂ [M-I]⁺, 336.1143, measured value: 336.1146.

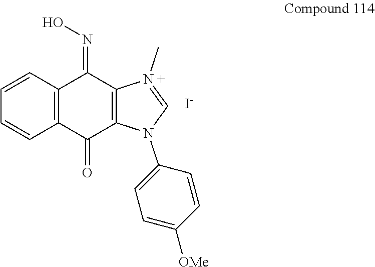

(E)-4-(hydroxylimino)-1-(4-methoxyphenyl)-3-methyl-9-oxo-4,9-dihydro-1H-naphthol[2,3-d]imidazole-3-iodide

¹H NMR (500 MHz, DMSO-d₆) δ 14.58 (s, 1H), 9.92 (s, 1H), 9.27 (dd, J=10.0, 1.0 Hz, 1H), 8.24 (dd, J=10.0, 1.5 Hz, 1H), 8.01-7.93 (m, 1H), 7.84 (td, J=10.0, 1.5 Hz, 1H), 7.66 (d, J=10.0 Hz, 2H), 7.21 (d, J=10.0 Hz, 2H), 4.17 (s, 3H), 3.89 (s, 3H).

¹³C NMR (126 MHZ, DMSO-d₆) δ 172.93, 161.12, 144.32, 138.47, 135.44, 135.22, 131.94, 131.40, 131.08, 127.96, 127.42, 126.91, 126.64, 125.31, 114.85, 56.23, 39.30.

HRMS (ESI-TOF) m/z theoretical value: C₁₉H₁₆N₃O₃ [M-I]⁺, 334.1186, measured value: 334.1184.

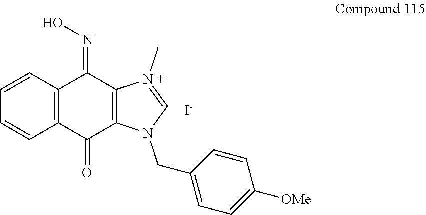

(E)-4-(hydroxylimino)-1-(4-methoxybenzyl)-3-methyl-9-oxo-4,9-dihydro-1H-naphthol[2,3-d]imidazole-3-iodide

¹H NMR (500 MHz, DMSO-d₆) δ 14.51 (s, 1H), 9.68 (s, 1H), 9.23 (dd, J=10.0, 1.0 Hz, 1H), 8.32 (dd, J=10.0, 1.0 Hz, 1H), 7.99-7.91 (m, 1H), 7.83 (td, J=10.0, 1.0 Hz, 1H), 7.52-7.45 (m, 2H), 7.04-6.96 (m, 2H), 5.79 (s, 2H), 4.10 (s, 3H), 3.76 (s, 3H).

¹³C NMR (126 MHz, DMSO-d₆) δ 174.06, 160.08, 143.40, 138.44, 136.09, 135.36, 131.88, 131.43, 130.78, 130.66, 127.41, 127.09, 126.36, 124.56, 114.74, 55.72, 51.49, 39.29.

HRMS (ESI-TOF) m/z theoretical value: C₂₀H₁₈N₃O₃ [M-I]⁺, 348.1343, measured value: 348.1347.

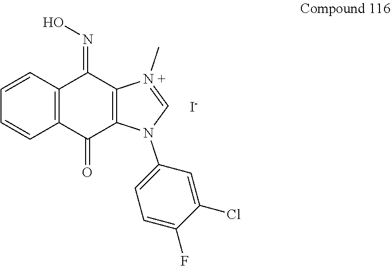

(E)-1-(3-chloro-4-fluorophenyl)-4-(hydroxylimino)-3-methyl-9-oxo-4,9-dihydro-1H-naphthol[2,3-d]imidazole-3-iodide

¹H NMR (500 MHZ, DMSO-d₆) δ 14.66 (s, 1H), 10.02 (s, 1H), 9.28 (dd, J=10.0, 1.0 Hz, 1H), 8.26 (dd, J=10.0, 1.5 Hz, 1H), 8.17 (dd, J=10.0, 2.5 Hz, 1H), 7.99 (td, J=5.0, 1.5 Hz, 1H), 7.88-7.77 (m, 3H), 4.20 (s, 3H).

¹³C NMR (126 MHz, DMSO-d₆) δ 173.01, 159.77 (157.78, J=250.0 Hz), 144.77, 138.33, 135.39, 135.23, 132.01, 131.44, 130.92, 130.75 (130.72, J=3.8 Hz), 129.46, 128.10 (1210.03, J=8.8 Hz), 127.49, 126.88, 125.30, 120.56 (120.40, J=20.0 Hz), 118.20 (118.02, J=22.5 Hz), 39.46.

HRMS (ESI-TOF) m/z theoretical value: C₁₈H₁₂ClFN₃O₂ [M-I]⁺, 356.0597, measured value: 356.0593.

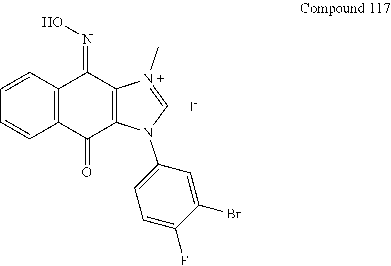

(E)-1-(3-bromo-4-fluorophenyl)-4-(hydroxylimino)-3-methyl-9-oxo-4,9-dihydro-1H-naphthol[2,3-d]imidazole-3-iodide

¹H NMR (400 MHZ, DMSO-d₆) δ 14.65 (s, 1H), 10.02 (s, 1H), 9.28 (dd, J=8.0, 1.2 Hz, 1H), 8.33-8.20 (m, 2H), 8.02-7.95 (m, 1H), 7.90-7.80 (m, 2H), 7.75 (t, J=8.0 Hz, 1H), 4.19 (s, 3H).

¹³C NMR (101 MHz, DMSO) δ 173.01, 161.09 (158.62, J=247.0 Hz), 144.79, 138.33, 135.38, 135.20, 132.12 (131.99, J=13.0 Hz), 131.44, 130.97, 130.93, 130.92, 128.70 (128.61, J=9.0 Hz), 127.48, 126.88, 125.32, 117.93 (117.69, J=24.0 Hz), 108.84 (108.62, J=22.0 Hz), 39.44.

HRMS (ESI-TOF) m/z theoretical value: C₁₈H₁₂BrFN₃O₂ [M-I]⁺, 400.0091, measured value: 400.0088.

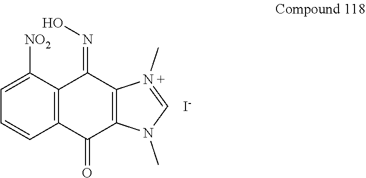

(E)-4-(hydroxylimino)-1,3-dimethyl-5-nitro-9-oxo-4,9-dihydro-1H-naphthol[2,3-d]imidazole-3-iodide

¹H NMR (400 MHZ, DMSO-d₆) δ 14.67 (s, 1H), 9.67 (s, 1H), 8.41 (d, J=7.8 Hz, 1H), 8.30 (d, J=7.9 Hz, 1H), 8.19 (t, J=7.8 Hz, 1H), 4.14 (d, J=21.7 Hz, 6H).

HRMS (ESI-TOF) m/z theoretical value: C₁₈H₁₂BrFN₃O₂ [M-I]⁺, 287.0775, measured value: 287.0778.

### Example 12

Compound 1 (0.05 mmol) and 310 μL of iodomethane were added to 3 mL of acetonitrile for reaction for 48 h at 70° C. At the end of the reaction, solvent was removed to obtain a yellow solid, and the solid was pulped for half an hour and subjected to suction filtration to obtain the final product.

Different synthetic intermediate compounds above were used to prepare Compounds 119-126 according to the method in Example 12; other materials and reaction conditions for the compounds were kept the same and raw materials could be purchased commercially:

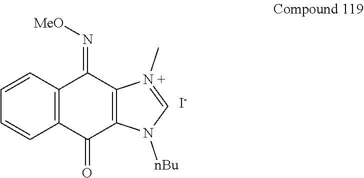

(E) and (Z)-1-butyl-4-(methoxylimino)-3-methyl-9-oxo-4,9-dihydro-1H-naphthol[2,3-d]imidazole-3-iodide

¹H NMR (500 MHz, DMSO-d₆) δ 9.65 (s, 1H), 9.02 (dd, J=5.0, 0.5 Hz, 1H), 8.35 (dd, J=10.0, 1.0 Hz, 1H), 7.99-7.92 (m, 1H), 7.88 (td, J=5.0, 1.0 Hz, 1H), 4.60 (t, J=7.0 Hz, 2H), 4.41 (s, 3H), 4.13 (s, 3H), 1.93-1.81 (m, 2H), 1.43-1.35 (m, 2H), 0.95 (t, J=5.0 Hz, 3H).

¹³C NMR (126 MHz, DMSO-d₆) δ 173.98, 143.58, 138.41, 135.35, 134.86, 132.54, 131.66, 131.22, 127.73, 126.70, 125.33, 66.37, 49.06, 39.25, 31.67, 19.28, 13.86.

HRMS (ESI-TOF) m/z theoretical value: C₁₇H₂₀N₃O₂ [M-I]⁺, 298.1550, measured value: 298.1555.

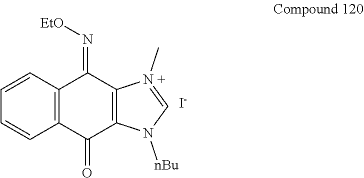

(E) and (Z)-1-butyl-4-(ethoxylimino)-3-methyl-9-oxo-4,9-dihydro-1H-naphthol[2,3-d]imidazole-3-iodide yellow solid, m. p. 134-136° C.

¹H NMR (500 MHZ, DMSO-d₆) δ 9.67 (s, 1H), 9.05 (d, J=10.0 Hz, 1H), 8.34 (dd, J=10.0, 1.5 Hz, 1H), 7.98 (td, J=10.0 1.5 Hz, 1H), 7.88 (td, J=10.0, 1.0 Hz, 1H), 4.67 (q, J=7.0 Hz, 2H), 4.60 (t, J=7.0 Hz, 2H), 4.13 (s, 3H), 1.94-1.82 (m, 2H), 1.50 (t, J=7.0 Hz, 3H), 1.42-1.33 (m, 2H), 0.95 (t, J=7.5 Hz, 3H).

¹³C NMR (126 MHz, DMSO-d₆) δ 173.97, 143.54, 138.27, 135.36, 135.06, 132.45, 131.55, 131.19, 127.70, 126.73, 125.24, 74.48, 49.06, 39.33, 31.67, 19.28, 15.04, 13.87.

HRMS (ESI-TOF) m/z theoretical value: C₁₈H₂₂N₃O₂ [M-I]⁺, 312.1707, measured value: 312.1707.

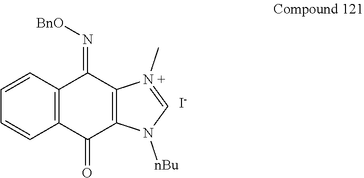

(E) and (Z)-4-((benzyloxy)imino)-1-butyl-3-methyl-9-oxo-4,9-dihydro-1H-naphthol[2,3-d]imidazole-3-iodide yellow solid, m. p. 122-124° C.

¹H NMR (500 MHz, DMSO-d₆) δ 9.62 (s, 1H), 9.09 (d, J=10.0 Hz, 1H), 8.34 (dd, J=10.0, 1.0 Hz, 1H), 8.01-7.94 (m, 1H), 7.88 (td, J=10.0, 1.0 Hz, 1H), 7.61-7.54 (m, 2H), 7.48-7.37 (m, 3H), 5.68 (s, 2H), 4.57 (t, J=7.0 Hz, 2H), 4.03 (s, 3H), 1.90-1.77 (m, 2H), 1.41-1.32 (m, 2H), 0.97-0.90 (m, 3H).

¹³C NMR (126 MHZ, DMSO) δ 173.94, 143.58, 138.73, 136.71, 135.39, 134.89, 132.57, 131.60, 131.25, 129.47, 129.21, 129.18, 127.76, 126.70, 125.35, 80.08, 49.03, 39.33, 31.66, 19.24, 13.85.

HRMS (ESI-TOF) m/z theoretical value: C₂₃H₂₃N₃O₂ [M-I]⁺, 374.1863, measured value: 374.1863.

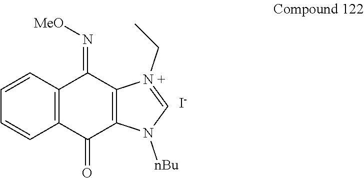

(E) and (Z)-1-butyl-3-ethyl-4-(methoxylimino)-9-oxo-4,9-dihydro-1H-naphthol[2,3-d]imidazole-3-iodide

yellow solid, m. p. 135-137° C.

¹H NMR (500 MHZ, DMSO-d₆) δ 9.71 (s, 1H), 9.02 (d, J=10.0 Hz, 1H), 8.34 (d, J=10.0, 1H), 7.97 (td, J=10.0, 1.5 Hz, 1H), 7.88 (t, J=10.0, 1H), 4.65-4.55 (m, 5H), 4.41 (s, 3H), 1.92-1.83 (m, 3H), 1.54 (t, J=7.0 Hz, 3H), 1.47-1.35 (m, 3H), 0.95 (t, J=7.0 Hz, 4H).

¹³C NMR (126 MHz, DMSO-d₆) δ 174.09, 142.86, 138.14, 135.34, 134.19, 132.51, 131.70, 131.11, 127.70, 126.81, 125.71, 66.27, 49.20, 47.07, 31.65, 19.35, 14.86, 13.89.

HRMS (ESI-TOF) m/z theoretical value: C₁₈H₂₂N₃O₂ [M-I]⁺, 312.1707, measured value: 312.1708.

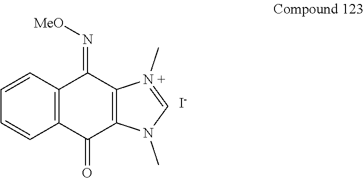

(E) and (Z)-4-(methoxylimino)-1,3-dimethyl-9-oxo-4,9-dihydro-1H-naphthol[2,3-d]imidazole-3-iodide

yellow solid, m. p. 167-169° C.

¹H NMR (400 MHZ, DMSO-d₆) δ 9.58 (s, 1H), 9.01 (d, J=8.0, 1H), 8.33 (dd, J=4.0, 1.2 Hz, 1H), 7.97 (td, J=8.0, 1.6 Hz, 1H), 7.88 (td, J=8.0, 1.2 Hz, 1H), 4.41 (s, 3H), 4.18 (s, 3H), 4.13 (s, 3H).

¹³C NMR (101 MHZ, DMSO) δ 174.16, 144.05, 138.25, 135.38, 134.26, 132.56, 131.72, 131.10, 127.63, 126.76, 125.85, 66.37, 39.08, 36.44.

HRMS (ESI-TOF) m/z theoretical value: C₁₄H₁₄N₃O₂ [M-I]⁺, 256.1081, measured value: 256.1085.

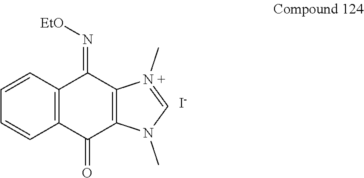

(E) and (Z)-4-(ethoxylimino)-1,3-dimethyl-9-oxo-4,9-dihydro-1H-naphthol[2,3-d]imidazole-3-iodide

yellow solid, m. p. 165-167° C.

¹H NMR (500 MHZ, DMSO-d₆) δ 9.57 (s, 1H), 9.06 (dd, J=10.0, 1.0 Hz, 1H), 8.34 (dd, J=10.0, 1.0 Hz, 1H), 7.98 (td, J=10.0, 1.5 Hz, 1H), 7.88 (td, J=5.0, 1.0 Hz, 1H), 4.67 (q, J=7.0 Hz, 2H), 4.18 (s, 3H), 4.13 (s, 3H), 1.50 (t, J=7.0 Hz, 3H).

¹³C NMR (126 MHz, DMSO-d₆) δ 174.18, 144.02, 138.15, 135.39, 134.49, 132.49, 131.62, 131.11, 127.63, 126.81, 125.80, 74.48, 39.12, 36.43, 15.02.

HRMS (ESI-TOF) m/z theoretical value: C₁₅H₁₅N₃O₂ [M-I]⁺, 270.1243, measured value: 270.1244.

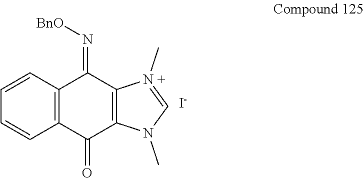

(E)-4-((benzyloxy)imino)-1,3-dimethyl-9-oxo-4,9-dihydro-1H-naphthol[2,3-d]imidazole-3-iodide

yellow solid, m. p. 177-179° C.

¹H NMR (500 MHZ, DMSO-d₆) δ 9.53 (s, 1H), 9.08 (dd, J=10.0, 1.0 Hz, 1H), 8.33 (dd, J=10.0, 1.0 Hz, 1H), 8.01-7.93 (m, 1H), 7.88 (td, J=10.0, 1.0 Hz, 1H), 7.60-7.55 (m, 2H), 7.49-7.38 (m, 3H), 5.68 (s, 2H), 4.16 (s, 3H), 4.03 (s, 3H).

¹³C NMR (126 MHZ, DMSO-d₆) δ 174.15, 144.04, 138.61, 136.66, 135.42, 134.32, 132.61, 131.66, 131.16, 129.49, 129.23, 129.19, 127.67, 126.77, 125.90, 80.09, 39.13, 36.39.

(Z)-4-((benzyloxy)imino)-1,3-dimethyl-9-oxo-4,9-dihydro-1H-naphthol[2,3-d]

¹H NMR (400 MHZ, DMSO-d₆) δ 9.57 (s, 1H), 8.22 (d, J=8.1 Hz, 1H), 8.15 (d, J=7.8 Hz, 1H), 7.86 (t, J=7.7 Hz, 1H), 7.75 (t, J=7.6 Hz, 1H), 7.54 (d, J=7.1 Hz, 2H), 7.41 (d, J=8.2 Hz, 3H), 5.57 (s, 2H), 4.13 (s, 3H), 4.00 (s, 3H).

(E) and (Z)-4-((benzyloxy)imino)-1,3-dimethyl-9-oxo-4,9-dihydro-1H-naphthol[2,3-d]imidazole-3-iodide

¹H NMR (400 MHZ, DMSO-d₆) δ 9.55 (s, 3H), 9.50 (s, 1H), 9.04 (d, J=8.0 Hz, 1H), 8.29 (dd, J=8.0, 1.6 Hz, 1H), 8.20 (dd, J=8.0, 1.2 Hz, 3H), 8.13 (dd, J=7.8, 1.4 Hz, 3H), 7.97-7.90 (m, 1H), 7.84 (td, J=7.6, 1.6 Hz, 4H), 7.73 (td, J=7.6, 1.2 Hz, 3H), 7.59-7.48 (m, 8H), 7.47-7.33 (m, 12H), 5.64 (s, 2H), 5.55 (s, 6H), 4.11 (s, 3H), 4.11 (s, 6H), 3.99 (s, 3H), 3.98 (s, 6H).

HRMS (ESI-TOF) m/z theoretical value: C₂₀H₁₈N₃O₂ [M-I]⁺, 332.1394, measured value: 332.1402.

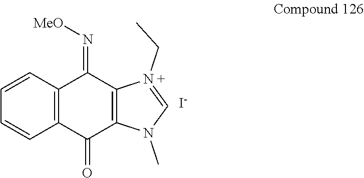

(E) and (Z)-3-ethyl-4-(methoxylimino)-1-methyl-9-oxo-4,9-dihydro-1H-naphthol[2,3-d]imidazole-3-iodide

yellow solid, m. p. 162-164° C.

¹H NMR (500 MHZ, DMSO-d₆) δ 9.57 (s, 1H), 9.06 (d, J=10.0 Hz, 1H), 8.34 (dd, J=10.0, 2.0 Hz, 1H), 7.98 (td, J=10.0, 2.0 Hz, 1H), 7.88 (td, J=10.0, 1.5 Hz, 1H), 4.67 (q, J=7.0 Hz, 2H), 4.18 (s, 3H), 4.13 (s, 3H), 1.50 (t, J=7.0 Hz, 3H).

¹³C NMR (126 MHz, DMSO-d₆) δ 174.18, 144.02, 138.15, 135.39, 134.49, 132.49, 131.62, 131.11, 127.63, 126.81, 125.80, 74.48, 39.12, 36.43, 15.02.

HRMS (ESI-TOF) m/z theoretical value: C₁₅H₁₆N₃O₂ [M-I]⁺, 270.1237, measured value: 270.1241.

### Example 13

Compound 1 (0.05 mmol) and 150 μL of iodomethane were added to 3 mL of acetonitrile for reaction for 8 h at 100° C. At the end of the reaction, solvent was removed to obtain a yellow solid, and the solid was pulped for half an hour and subjected to suction filtration to obtain the final product.

Different synthetic intermediate compounds above were used to prepare Compounds 127-151 according to the method in Example 13; other materials and reaction conditions for the compounds were kept the same and raw materials could be purchased commercially:

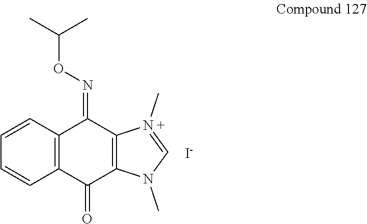

(E) and (Z)-4-(isopropoxyimino)-1,3-dimethyl-9-oxo-4,9-dihydro-1H-naphthol[2,3-d]imidazole-3-iodide

yellow solid, m. p. 187-189° C.

¹H NMR (400 MHZ, DMSO-d₆) δ 9.58 (s, 1H), 9.07 (dd, J=8.0, 0.4 Hz, 1H), 8.34 (dd, J=8.0, 1.2 Hz, 1H), 8.03-7.95 (m, 1H), 7.88 (td, J=8.0, 1.2 Hz, 1H), 4.92-4.82 (m, 1H), 4.18 (s, 3H), 4.14 (s, 3H), 1.51 (d, J=8.0 Hz, 6H).

¹³C NMR (101 MHZ, DMSO-d₆) δ 174.16, 143.98, 137.79, 135.40, 134.67, 132.39, 131.52, 131.09, 127.60, 126.84, 125.73, 81.42, 39.23, 36.45, 21.77.

HRMS (ESI-TOF) m/z theoretical value: C₁₆H₁₈N₃O₂ [M-I]⁺, 284.1394, measured value: 284.1399.

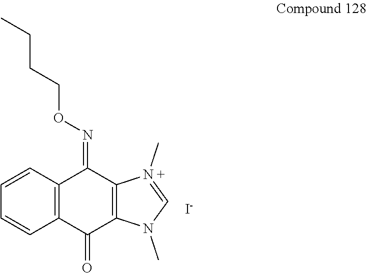

(E) and (Z)-4-(butoxyimino)-1,3-dimethyl-9-oxo-4,9-dihydro-1H-naphthol[2,3-d]

yellow solid, m. p. 201-203° C.

¹H NMR (500 MHZ, DMSO-d₆) δ 9.59 (s, 1H), 9.03 (dd, J=8.0, 1.0 Hz, 1H), 8.34 (dd, J=8.0, 1.5 Hz, 1H), 8.02-7.93 (m, 1H), 7.90-7.85 (m, 1H), 4.64 (t, J=6.5 Hz, 2H), 4.18 (s, 3H), 4.12 (s, 3H), 1.95-1.77 (m, 2H), 1.53-1.42 (m, 2H), 0.98 (t, J=7.5 Hz, 3H).

¹³C NMR (126 MHz, DMSO-d₆) δ 174.16, 144.02, 138.15, 135.43, 134.46, 132.49, 131.48, 131.10, 127.64, 126.80, 125.78, 78.53, 39.11, 36.43, 31.07, 19.09, 14.20.

HRMS (ESI-TOF) m/z theoretical value: C₁₇H₂₀N₃O₂ [M-I]⁺, 298.1550, measured value: 298.1551.

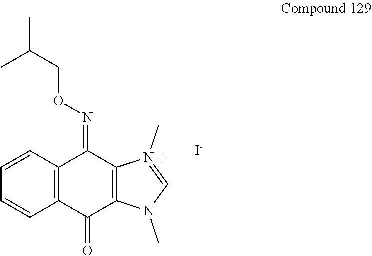

(E) and (Z)-4-(isobutoxyimino)-1,3-dimethyl-9-oxo-4,9-dihydro-1H-naphthol[2,3-d]imidazole-3-iodide

yellow solid, m. p. 139-141° C.

¹H NMR (500 MHZ, DMSO-d₆) δ 9.57 (s, 1H), 9.03 (J=10.0 Hz, 1H), 8.35 (dd, J=10.0, 1.5 Hz, 1H), 8.05-7.98 (m, 1H), 7.89 (t, J=7.5 Hz, 1H), 4.44 (d, J=6.5 Hz, 2H), 4.18 (s, 3H), 4.12 (s, 3H), 2.30-2.15 (m, 1H), 1.05 (d, J=5.0 Hz, 6H).

¹³C NMR (126 MHz, DMSO-d₆) δ 174.16, 144.03, 138.16, 135.45, 134.47, 132.51, 131.41, 131.13, 127.69, 126.82, 125.81, 84.91, 39.11, 36.43, 28.32, 19.37.

HRMS (ESI-TOF) m/z theoretical value: C₁₇H₂₀N₃O₂ [M-I]⁺, 298.1550, measured value: 298.1551.

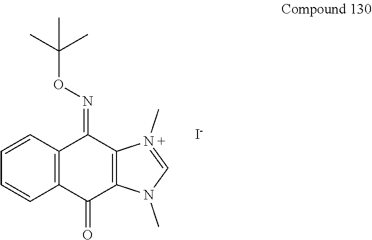

(E) and (Z)-4-(tert-butoxyimino)-1,3-dimethyl-9-oxo-4,9-dihydro-1H-naphthol[2,3-d]imidazole-3-iodide

yellow solid, m. p. 221-223° C.

¹H NMR (500 MHZ, DMSO-d₆) δ 9.59 (s, 1H), 9.10 (dd, J=10.0 Hz, 1H), 8.35 (dd, J=5.0, 1.5 Hz, 1H), 8.02-7.95 (m, 1H), 7.91-7.85 (m, 1H), 4.19 (s, 3H), 4.15 (s, 3H), 1.56 (s, 9H).

¹³C NMR (126 MHz, DMSO-d₆) δ 174.19, 143.95, 137.64, 135.39, 134.97, 132.32, 131.49, 131.12, 127.59, 126.94, 125.70, 85.60, 39.44, 36.44, 27.70, 27.65.

HRMS (ESI-TOF) m/z theoretical value: C₁₇H₂₀N₃O₂ [M-I]⁺, 298.1550, measured value: 298.1551.

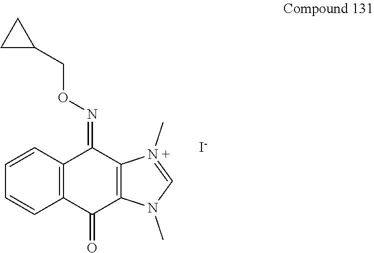

(E) and (Z)-4-((cyclopropylmethoxy)imino)-1,3-dimethyl-9-oxo-4,9-dihydro-1H-naphthol[2,3-d]imidazole-3-iodide

yellow solid, m. p. 170-172° C.

¹H NMR (400 MHZ, DMSO-d₆) δ 9.58 (s, 1H), 9.09 (dd, J=8.0, 0.4 Hz, 1H), 8.34 (dd, J=8.0, 1.6 Hz, 1H), 8.03-7.95 (m, 1H), 7.88 (td, J=8.0, 1.2 Hz, 1H), 4.47 (d, J=8.0 Hz, 2H), 4.18 (s, 4H), 4.13 (s, 3H), 1.47-1.35 (m, 1H), 0.70-0.62 (m, 2H), 0.51-0.42 (m, 2H).

¹³C NMR (101 MHz, DMSO-d₆) δ 174.17, 144.01, 1310.08, 135.38, 134.48, 132.46, 131.56, 131.11, 127.62, 126.88, 125.79, 83.19, 39.14, 36.44, 10.70, 3.76.

HRMS (ESI-TOF) m/z theoretical value: C₁₇H₁₈N₃O₂ [M-I]⁺, 296.1394, measured value: 296.1397.

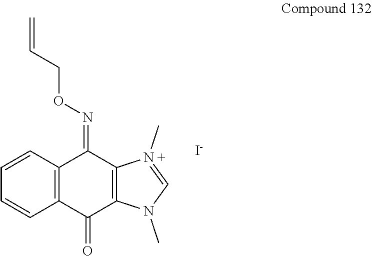

(E) and (Z)-4-((allyloxy)imino)-1,3-dimethyl-9-oxo-4,9-dihydro-1H-naphthol[2,3-d]imidazole-3-iodide

yellow solid, m. p. 139-141° C.

¹H NMR (400 MHZ, DMSO-d₆) δ 9.57 (s, 1H), 9.04 (dd, J=8.0, 1.2 Hz, 1H), 8.34 (dd, J=8.0, 1.2 Hz, 1H), 7.99 (td, J=8.0, 1.2 Hz, 1H), 7.89 (td, J=8.0, 1.2 Hz, 1H), 6.28-6.15 (m, 1H), 5.58-5.50 (m, 1H), 5.46-5.39 (m, 1H), 5.12 (d, J=8.0 Hz, 2H), 4.18 (s, 3H), 4.10 (s, 3H).

¹³C NMR (101 MHz, DMSO-d₆) δ 174.16, 144.07, 138.48, 135.41, 134.38, 133.51, 132.59, 131.66, 131.13, 127.67, 126.74, 125.88, 120.67, 79.10, 39.17, 36.44.

HRMS (ESI-TOF) m/z theoretical value: C₁₆H₁₆N₃O₂ [M-I]⁺, 282.1237, measured value: 282.1239.

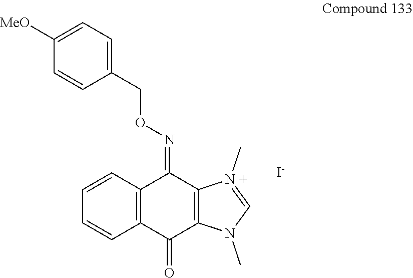

(E) and (Z)-4-(((4-methoxybenzyl)oxy)imino)-1,3-dimethyl-9-oxo-4,9-dihydro-1H-naphthol[2,3-d]imidazole-3-iodide

¹H NMR (400 MHZ, DMSO-d₆) δ 9.54 (s, 1H), 9.05 (dd, J=10.0, 0.8 Hz, 1H), 8.32 (dd, J=8.0, 1.2 Hz, 1H), 8.02-7.92 (m, 1H), 7.86 (td, J=8.0, 1.2 Hz, 1H), 7.56-7.49 (m, 2H), 7.03-6.95 (m, 2H), 5.60 (s, 2H), 4 . . . 16 (s, 3H), 4.06 (s, 3H), 3.77 (s, 3H).

¹³C NMR (101 MHz, DMSO-d₆) δ 174.13, 160.14, 144.02, 138.38, 135.38, 134.37, 132.53, 131.58, 131.44, 131.11, 128.50, 127.63, 126.79, 125.83, 114.51, 79.91, 55.65, 39.22, 36.41.

HRMS (ESI-TOF) m/z theoretical value: C₂₁H₂₀N₃O₃ [M-I]⁺, 363.11499, measured value: 363.1499.

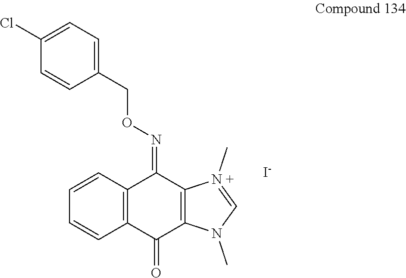

(E) and (Z)-4-(((4-chlorobenzyl)oxy)imino)-1,3-dimethyl-9-oxo-4,9-dihydro-1H-naphthol[2,3-d]imidazole-3-iodide

¹H NMR (400 MHZ, DMSO-d₆) δ 9.54 (s, 1H), 9.07 (dd, J=8.0, 0.8 Hz, 1H), 8.33 (dd, J=8.0, 1.6 Hz, 1H), 8.01-. 95 (m, 1H), 7.88 (td, J=8.0, 1.2 Hz, 1H), 7.65-7.58 (m, 2H), 7.55-7.48 (m, 2H), 5.67 (s, 2H), 4.16 (s, 3H), 4.02 (s, 3H).

¹³C NMR (101 MHZ, DMSO-d₆) δ 174.14, 144.05, 138.81, 135.74, 135.43, 134.25, 133.88, 132.66, 131.75, 131.41, 131.16, 129.19, 127.68, 126.73, 125.93, 78.99, 39.17, 36.41.

HRMS (ESI-TOF) m/z theoretical value: C₂₀H₁₇ClN₃O₂ [M-I]⁺, 366.1004, measured value: 366.1006.

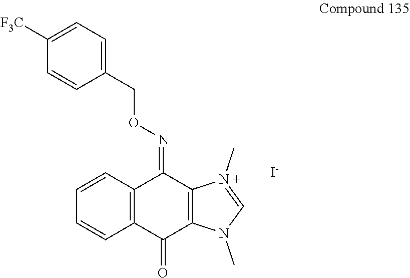

(E) and (Z)-1,3-dimethyl-9-oxo-4-(((4-(trifluoromethyl)benzyl)oxy)imino)-4,9-dihydro-1H-naphthol[2,3-d]imidazole-3-iodide

yellow solid, m. p. 158-160° C.

¹H NMR (400 MHZ, DMSO-d₆) δ 9.53 (s, 1H), 9.11 (dd, J=8.0, 0.8 Hz, 1H), 8.34 (dd, J=8.0, 0.8 Hz, 1H), 7.99 (td, J=8.0, 1.6 Hz, 1H), 7.89 (td, J=8.0, 1.2 Hz, 1H), 7.86-7.76 (m, 4H), 5.78 (s, 2H), 4.16 (s, 3H), 4.00 (s, 3H).

¹³C NMR (101 MHz, DMSO-d₆) δ 174.14, 144.07, 141.53, 139.03, 135.46, 134.20, 132.73, 131.84, 131.19, 129.63, 129.32, 127.71, 126.72, 126.11 (126.08, 126.04, 126.00, J=4.0 Hz), 123.26, 78.84, 39.12, 36.41.

HRMS (ESI): m/z theoretical value: C₂₁H₁₇F₃N₃O₂ [M-I]⁺, 400.1267, measured value: 400.1268.

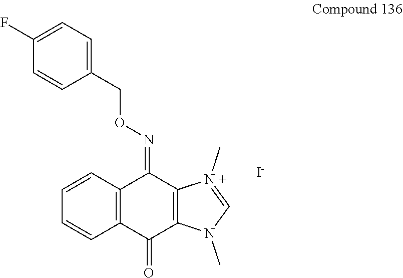

(E) and (Z)-4-(((4-fluorobenzyl)oxy)imino)-1,3-dimethyl-9-oxo-4,9-dihydro-1H-naphthol[2,3-d]imidazole-3-iodide

yellow solid, m. p. 175-177° C.

¹H NMR (400 MHZ, DMSO-d₆) δ 9.54 (s, 1H), 9.07 (d, J=8.0 Hz, 1H), 8.32 (dd, J=8.0, 1.2 Hz, 1H), 7.97 (td, J=8.0, 1.6 Hz, 1H), 7.88 (td, J=8.0, 1.2 Hz, 1H), 7.69-7.56 (m, 2H), 7.35-7.24 (m, 2H), 5.66 (s, 2H), 4.16 (s, 3H), 4.03 (s, 3H).

¹³C NMR (101 MHZ, DMSO-d₆) δ 174.14, 163.99 (161.55, J=244.0 Hz), 144.04, 138.68, 135.40, 134.29, 133.02 (132.99, J=3.0 Hz), 132.62, 131.98 (131.89, J=9.0 Hz), 131.70, 131.15, 127.66, 126.75, 125.91, 116.14 (115.93, J=21.0 Hz), 79.14, 39.18, 36.41.

HRMS (ESI-TOF) m/z theoretical value: C₂₀H₁₇FN₃O₂ [M-I]⁺, 350.1299, measured value: 350.1302.

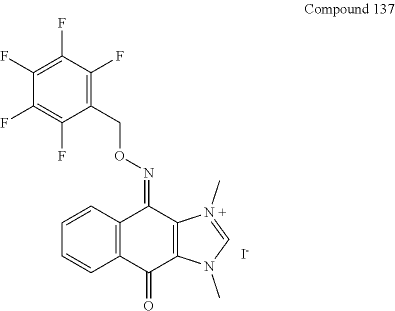

(E) and (Z)-1,3-dimethyl-9-oxo-4-(((perfluorophenyl) methoxy)imino)-4,9-dihydro-1H-naphthol[2,3-d]imidazole-3-iodide

¹H NMR (400 MHZ, DMSO-d₆) δ 9.54 (s, 1H), 8.94 (d, J=8.0 Hz, 1H), 8.30 (d, J=8.0 Hz, 1H), 8.02-7.92 (m, 1H), 7.87 (t, J=8.0 Hz, 1H), 5.78 (s, 2H), 4.14 (s, 3H), 4.03 (s, 3H).

¹³C NMR (126 MHz, DMSO-d₆) δ 175.44, 174.10, 144.09, 139.74, 135.84, 135.44, 133.92, 132.92, 132.27, 131.84, 131.26, 127.79, 127.39, 126.66, 126.23, 66.49, 38.71, 36.47.

HRMS (ESI): m/z theoretical value: C₂₀H₁₃F₅N₃O₂ [M-I]⁺, 422.0922, measured value: 422.0920.

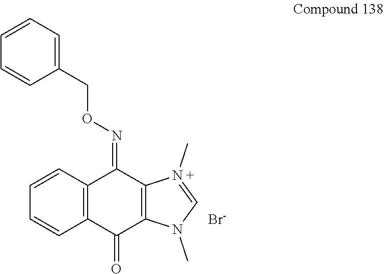

(E) and (Z)-4-((benzyloxy)imino)-1,3-dimethyl-9-oxo-4,9-dihydro-1H-naphthol[2,3-d]imidazole-3-iodide

¹H NMR (500 MHZ, DMSO-d₆) δ 9.53 (s, 1H), 9.08 (dd, J=10.0, 1.0 Hz, 1H), 8.33 (dd, J=10.0, 1.0 Hz, 1H), 8.01-7.93 (m, 1H), 7.88 (td, J=10.0, 1.0 Hz, 1H), 7.60-7.55 (m, 2H), 7.49-7.38 (m, 3H), 5.68 (s, 2H), 4.16 (s, 3H), 4.03 (s, 3H).

¹³C NMR (126 MHz, DMSO-d₆) δ 174.15, 144.04, 138.61, 136.66, 135.42, 134.32, 132.61, 131.66, 131.16, 129.49, 129.23, 129.19, 127.67, 126.77, 125.90, 80.09, 39.13, 36.39.

HRMS (ESI-TOF) m/z theoretical value: C₂₀H₁₈N₃O₂ [M-I]⁺, 322.1394, measured value: 322.1393.

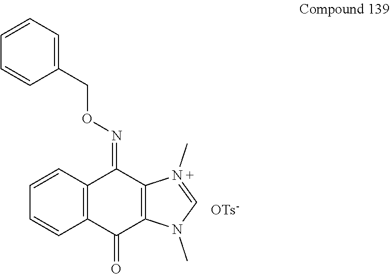

(E) and (Z)-4-((benzyloxy)imino)-1,3-dimethyl-9-oxo-4,9-dihydro-1H-naphthol[2,3-d]imidazole-3-p-toluenesulfonamide

¹H NMR (500 MHZ, DMSO-d₆) δ 9.53 (s, 1H), 9.08 (dd, J=10.0, 1.0 Hz, 1H), 8.33 (dd, J=10.0, 1.0 Hz, 1H), 8.01-7.93 (m, 1H), 7.88 (td, J=10.0, 1.0 Hz, 1H), 7.60-7.55 (m, 2H), 7.49-7.38 (m, 3H), 5.68 (s, 2H), 4.16 (s, 3H), 4.03 (s, 3H).

¹³C NMR (126 MHZ, DMSO-d₆) δ 174.15, 144.04, 138.61, 136.66, 135.42, 134.32, 132.61, 131.66, 131.16, 129.49, 129.23, 129.19, 127.67, 126.77, 125.90, 80.09, 39.13, 36.39.

HRMS (ESI-TOF) m/z theoretical value: C₂₀H₁₈N₃O₂ [M-I]⁺, 322.1394, measured value: 322.1393.

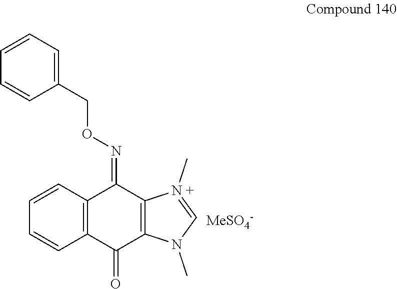

(E) and (Z)-4-((benzyloxy)imino)-1,3-dimethyl-9-oxo-4,9-dihydro-1H-naphthol[2,3-d]imidazole-3-dimethyl sulfate

¹H NMR (500 MHZ, DMSO-d₆) δ 9.53 (s, 1H), 9.08 (dd, J=10.0, 1.0 Hz, 1H), 8.33 (dd, J=10.0, 1.0 Hz, 1H), 8.01-7.93 (m, 1H), 7.88 (td, J=10.0, 1.0 Hz, 1H), 7.60-7.55 (m, 2H), 7.49-7.38 (m, 3H), 5.68 (s, 2H), 4.16 (s, 3H), 4.03 (s, 3H).

¹³C NMR (126 MHz, DMSO-d₆) δ 174.15, 144.04, 138.61, 136.66, 135.42, 134.32, 132.61, 131.66, 131.16, 129.49, 129.23, 129.19, 127.67, 126.77, 125.90, 80.09, 39.13, 36.39.

HRMS (ESI-TOF) m/z theoretical value: C₂₀H₁₈N₃O₂ [M-I]⁺, 322.1394, measured value: 322.1395.

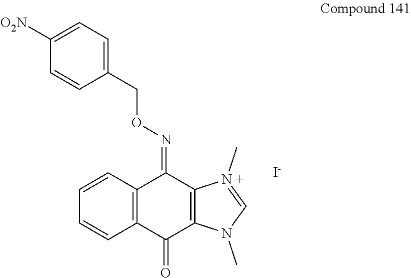

(E) and (Z)-1,3-dimethyl-4-(((4-nitrobenzyl)oxy)imino)-9-oxo-4,9-dihydro-1H-naphthol[2,3-d]imidazole-3-iodide

¹H NMR (500 MHZ, DMSO-d₆) δ 9.54 (s, 1H), 9.11 (d, J=10.0 Hz, 1H), 8.35-8.28 (m, 3H), 8.00 (td, J=10.0, 1.5 Hz, 1H), 7.90 (td, J=10.0, 1.0 Hz, 1H), 7.84 (d, J=10.0 Hz, 2H), 5.83 (s, 2H), 4.16 (s, 3H), 3.98 (s, 3H).

¹³C NMR (126 MHz, DMSO-d₆) δ 174.14, 147.97, 144.44, 144.09, 139.22, 135.47, 134.14, 132.79, 131.92, 131.21, 130.21, 127.74, 126.70, 126.04, 124.28, 78.38, 39.14, 36.44.

HRMS (ESI-TOF) m/z theoretical value: C₂₀H₁₇N₄O₄ [M-I]⁺, 377.1244, measured value: 377.1247.

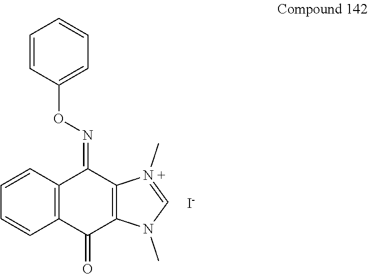

(E) and (Z)-4-((phenoxy)imino)-1,3-dimethyl-9-oxo-4,9-dihydro-1H-naphthol[2,3-d]imidazole-3-iodide

¹H NMR (400 MHZ, DMSO-d₆) δ 9.62 (s, 3H), 9.56 (s, 1H), 9.07 (d, J=8.0 Hz, 1H), 8.35 (dd, J=8.0, 1.6 Hz, 1H), 8.29 (dd, J=8.0, 1.2 Hz, 3H), 8.18 (dd, J=7.8, 1.4 Hz, 3H), 8.03-7.95 (m, 1H), 7.89 (td, J=7.6, 1.6 Hz, 4H), 7.79 (td, J=7.6, 1.2 Hz, 3H), 7.69-7.58 (m, 8H), 7.49-7.36 (m, 12H), 4.14 (s, 3H), 4.13 (s, 6H), 4.02 (s, 3H), 4.00 (s, 6H).

HRMS (ESI-TOF) m/z theoretical value: C₂₀H₁₈N₃O₂ [M-I]⁺, 318.1237, measured value: 318.1238.

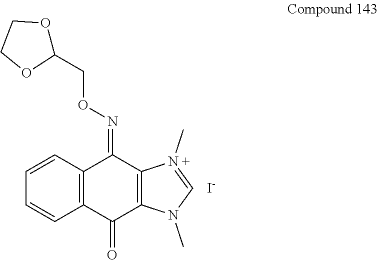

(E)-4-(((1,3-dioxoheterocycle-2-yl) methoxy)imino)-1,3-dimethyl-9-oxo-4,9-dihydro-1H-naphthol[2,3-d]imidazole-3-iodide

¹H NMR (400 MHZ, DMSO-d₆) δ 9.56 (s, 1H), 9.03 (d, J=8.0 Hz, 1H), 8.35 (d, J=8.0 Hz, 1H), 7.98 (t, J=8.0 Hz, 1H), 7.87 (t, J=8.0 Hz, 1H), 5.46 (t, J=4.0 Hz, 2H), 4.18 (s, 3H), 4.06 (s, 3H), 4.01 (t, J=4.0 Hz, 2H), 3.98 (s, J=4.0 Hz, 2H).

HRMS (ESI-TOF) m/z theoretical value: C₂₀H₁₈N₃O₂ [M-I]⁺, 328.1292, measured value: 328.1297.

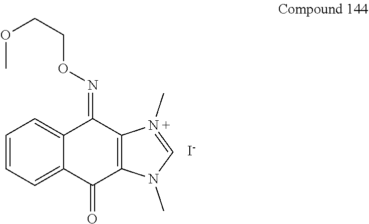

(E)-4-((2-methoxyethoxy)imino)-1,3-dimethyl-9-oxo-4,9-dihydro-1H-naphthol[2,3-d]imidazole-3-iodide

¹H NMR (400 MHZ, DMSO-d₆) δ 9.55 (s, 1H), 9.02 (d, J=8.0 Hz, 1H), 8.32 (d, J=8.0 Hz, 1H), 7.96 (t, J=8.0 Hz, 1H), 7.86 (t, J=8.0 Hz, 1H), 4.73 (t, J=4.0 Hz, 2H), 4.16 (s, 3H), 4.11 (s, 3H), 3.81 (t, J=4.0 Hz, 2H), 3.32 (s, 3H).

¹³C NMR (126 MHz, DMSO-d₆) δ 174.17, 144.02, 138.40, 135.39, 134.38, 132.57, 131.62, 131.14, 127.65, 126.78, 125.88, 77.73, 70.56, 58.77, 39.12, 36.45.

HRMS (ESI-TOF) m/z theoretical value: C₁₆H₁₈N₃O₃ [M-I]⁺, 300.1343, measured value: 300.1347.

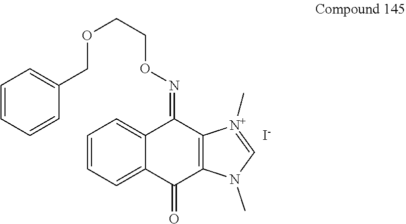 [SMILES: Cn1c[n+](C)c2c1C(=O)c1ccccc1/C2=N\OCCOCc1ccccc1 ; [I-]]

(E)-4-((2-(benzyloxy) ethoxy)imino)-1,3-dimethyl-9-oxo-4,9-dihydro-1H-naphthol[2,3-d]imidazole-3-iodide

¹H NMR (400 MHZ, DMSO-d₆) δ 9.53 (s, 1H), 9.04 (d, J=8.0 Hz, 1H), 8.32 (d, J=8.0 Hz, 1H), 7.94 (t, J=8.0 Hz, 1H), 7.87 (t, J=8.0 Hz, 1H), 7.34-7.19 (m, 4H), 4.76 (t, J=4.0 Hz, 2H), 4.56 (s, 2H), 4.16 (s, 3H), 4.03 (s, 3H), 3.90 (t, J=4.0 Hz, 2H).

¹³C NMR (126 MHz, DMSO-d₆) δ 174.17, 144.00, 138.69, 138.43, 135.32, 134.33, 132.59, 131.65, 131.15, 128.71, 1210.04, 127.98, 127.65, 126.79, 125.87, 77.75, 72.48, 68.19, 39.03, 36.46.

HRMS (ESI-TOF) m/z theoretical value: C₂₂H₂₂N₃O₃ [M-I]⁺, 376.1656, measured value: 376.1656.

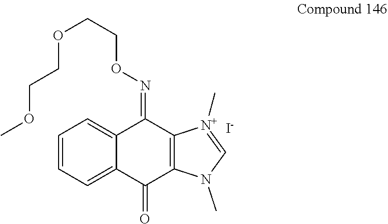 [SMILES: COCCOCCO/N=C1\c2ccccc2C(=O)c2c1[n+](C)cn2C ; [I-]]

(E)-4-((2-(2-methoxyethoxy) ethoxy)imino)-1,3-dimethyl-9-oxo-4,9-dihydro-1H-naphthol[2,3-d]imidazole-3-iodide

¹H NMR (400 MHZ, DMSO-d₆) δ 9.58 (s, 1H), 9.07 (dd, J=8.0, 0.8 Hz, 1H), 8.34 (dd, J=8.0, 1.2 Hz, 1H), 8.00-7.93 (m, 1H), 7.89 (td, J=8.0, 1.2 Hz, 1H), 4.82-4.69 (m, 2H), 4.18 (s, 3H), 4.13 (s, 3H), 3.95-3.86 (m, 2H), 3.68-3.59 (m, 2H), 3.53-3.44 (m, 2H), 3.23 (s, 3H).

¹³C NMR (101 MHz, DMSO-d₆) δ 174.17, 144.01, 138.40, 135.34, 134.37, 132.57, 131.68, 131.13, 127.62, 126.77, 125.86, 77.83, 71.77, 70.18, 69.12, 58.57, 39.08, 36.43.

HRMS (ESI-TOF) m/z theoretical value: C₁₈H₂₂N₃O₄ [M-I]⁺, 344.1605, measured value: 344.1609.

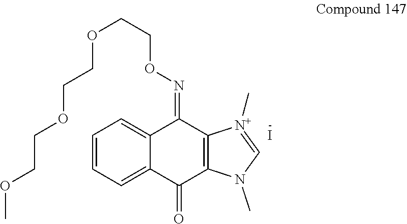

(E) and (Z)-4-((2-(2-(2-methoxyethoxy) ethoxy) ethoxy)imino)-1,3-dimethyl-9-oxo-4,9-dihydro-1H-naphthol[2,3-d]imidazole-3-iodide

¹H NMR (400 MHZ, DMSO-d₆) δ 9.63 (s, 1H), 9.56 (s, 1H), 9.02 (d, J=8.0 Hz, 1H), 8.34-8.09 (m, 3H), 8.01-7.70 (m, 4H), 4.78-4.58 (m, 4H), 4.21-4.01 (m, 11H), 3.90-3.80 (m, 4H), 3.63-3.42 (m, 10H), 3.40-3.30 (m, 5H), 3.16 (d, J=12.0 Hz, 5H).

¹³C NMR (101 MHz, DMSO) δ 175.23, 174.18, 144.73, 144.01, 138.39, 137.03, 135.84, 135.36, 134.81, 133.19, 132.56, 131.67, 131.13, 131.03, 130.53, 129.74, 128.12, 127.62, 127.38, 126.61, 125.85, 125.38, 77.84, 76.78, 71.73, 70.42, 70.26, 70.22, 70.10, 70.05, 70.01, 69.12, 68.72, 58.54, 58.50, 41.50, 39.09, 36.70, 36.62, 36.42.

HRMS (ESI-TOF) m/z theoretical value: C₂₀H₂₆N₃O₅ [M-I]⁺, 388.1867, measured value: 388.1866.

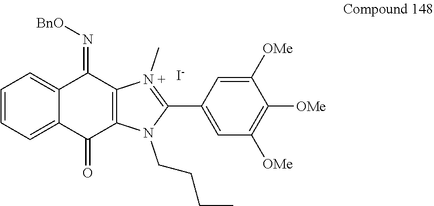

(E) and (Z)-4-((benzyloxy)imino)-1-butyl-3-methyl-9-oxo-2-(3,4,5-trimethoxyphenyl)-4,9-dihydro-1H-naphthol[2,3-d]imidazole-3-iodide

¹H NMR (400 MHZ, DMSO-d₆) δ 9.01 (d, J=8.1 Hz, 1H), 8.27 (dd, J=7.8, 1.6 Hz, 1H), 8.17-8.08 (m, 5H), 7.93-7.89 (m, 1H), 7.85-7.79 (m, 3H), 7.72 (t, J=7.4 Hz, 3H), 7.54-7.47 (m, 7H), 7.45-7.30 (m, 9H), 5.64 (s, 2H), 5.54 (s, 2H), 5.53 (s, 3H), 4.10 (d, J=3.4 Hz, 8H), 4.02 (s, 2H), 3.95 (s, 3H), 3.84 (s, 6H), 2.76 (s, 3H), 2.75 (s, 5H), 2.67 (s, 3H).

HRMS (ESI-TOF) m/z theoretical value: C₂₀H₂₆N₃O₅ [M-I]⁺, 540.2493, measured value: 540.2495.

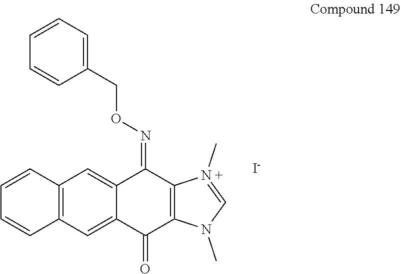

(E) and (Z)-4-((benzyloxy)imino)-1,3-dimethyl-11-oxo-4, 11-dihydro-1H-anthracene[2,3-d]imidazole-3-iodide

¹H NMR (400 MHZ, DMSO-d₆) δ 9.67 (s, 1H), 9.60 (s, 1H), 9.54 (s, 1H), 8.88 (s, 1H), 8.82 (s, 1H), 8.74 (s, 1H), 8.31-8.13 (m, 4H), 7.84-7.68 (m, 4H), 7.68-7.55 (m, 4H), 7.51-7.38 (m, 6H), 5.71 (s, 2H), 5.61 (s, 2H), 4.17 (m, 6H), 4.02 (s, 3H), 4.00 (s, 3H).

¹³C NMR (101 MHz, DMSO-d₆) δ 174.07, 144.77, 144.17, 138.54, 137.03, 136.19, 135.13, 135.08, 133.61, 132.73, 130.69, 130.54, 130.41, 130.37, 130.17, 130.10, 129.85, 129.75, 129.40, 129.26, 129.23, 129.17, 129.13, 129.04, 128.92, 128.47, 126.26, 125.23, 122.71, 79.71, 78.95.

HRMS (ESI-TOF) m/z theoretical value: C₂₀H₂₆N₃O₅ [M-I]⁺, 382.1550, measured value: 382.1555.

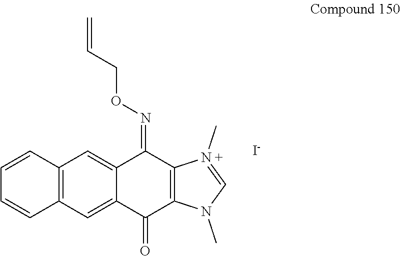

(E) and (Z)-4-((allyloxy)imino)-1,3-dimethyl-11-oxo-4,11-dihydro-1H-anthracene[2,3-d]imidazole-3-iodide

¹H NMR (400 MHZ, DMSO-d₆) δ 9.67 (s, 2H), 9.58 (s, 2H), 8.98 (s, 2H), 8.87 (s, 1H), 8.75 (s, 1H), 8.38-8.34 (m, 2H), 8.23 (dd, J=6.4, 2.8 Hz, 3H), 7.87-7.79 (m, 5H), 6.32-6.17 (m, 5H), 5.18 (dt, J=5.8, 1.2 Hz, 3H), 5.07 (d, J=6.0 Hz, 2H), 4.23-4.20 (m, 5H), 4.20 (s, 3H), 4.11 (d, J=1.6 Hz, 8H).

HRMS (ESI-TOF) m/z theoretical value: C₂₀H₂₆N₃O₅ [M-I]⁺, 332.1334, measured value: 332.1335.

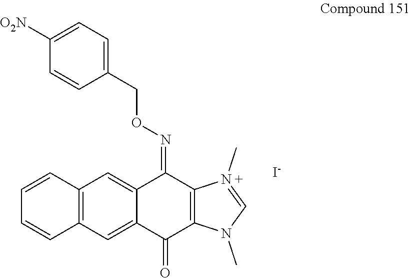

(E) and (Z)-1,3-dimethyl-4-(((4-nitrobenzyl)oxy)imino)-11-oxo-4,11-dihydro-1H-anthracene[2,3-d]imidazole-3-iodide

¹H NMR (400 MHZ, DMSO-d₆) δ 9.74 (s, 1H), 9.53 (s, 1H), 8.94 (s, 1H), 8.34-8.24 (m, 5H), 7.86-7.79 (m, 3H), 5.86 (s, 2H), 4.18 (s, 3H), 3.97 (s, 3H).

HRMS (ESI-TOF) m/z theoretical value: C₂₀H₂₆N₃O₅ [M-I]⁺, 427.1401, measured value: 427.1405.

Compounds obtained by the method of Examples 1-10 may be used to obtain the final imidazolium derivative in Examples 11-13. In addition, other similar quinonoxime imidazolium derivatives and synthetic intermediates thereof may be prepared by the method recited in the above examples or preparation methods, or prepared by some change methods commonly known by those skilled in the art.

Effects of the compounds in the present invention are confirmed by the following tests.

### Test Example 1. In Vitro Inhibition Test for Cancer Cell Proliferation

Test method: pancreatic cancer cells CFPAC-1 were cultured in a Dalbeco-improved Eagle medium with the addition of 10% FBS and 5% double-antibody. CFPAC-1 cancer cells were inoculated into a 96-well plate for incubation for 24 h; in the following day, test compounds having various concentrations of (100, 20, 10, 1, 0.1, 0.01, and 0.001 μM) and positive control agents were added per well; corresponding volume of fresh medium was added to blank control. After addition for 24 h or 48 h, cell proliferation was evaluated by chromogenic reaction of MTT formazan.

Test result: compounds of the present invention inhibit proliferation of cancer cells well; the median lethal concentration IC₅₀ value is below 1 μM, and the optimal compound may be up to 10 nM below.

| Compound | | | | |
|---|---|---|---|---|
| No. | IC₅₀(A549) | IC₅₀(HeLa) | IC₅₀(MCF-7) | IC₅₀(L929) |
| 91 | 0.53 | 0.67 | 0.84 | 4.70 |
| 92 | 0.54 | 1.63 | 0.94 | 13.47 |
| 93 | 1.03 | 2.18 | 1.43 | 32.73 |
| 94 | 0.67 | 1.43 | 0.84 | 39.23 |
| 95 | 1.89 | 18.3 | 2.00 | 26.28 |
| 96 | 2.43 | 9.70 | 1.26 | 57.38 |
| 97 | 1.37 | 1.97 | 1.05 | 74.02 |
| 98 | 0.59 | 2.18 | 1.81 | 38.90 |
| 99 | 1.72 | 5.74 | 1.63 | 19.8 |
| 100 | 0.62 | 2.18 | 0.87 | 6.64 |
| 101 | 0.64 | 1.80 | 0.87 | 13.05 |
| 102 | 0.63 | 1.05 | 0.89 | 37.35 |
| 103 | 0.60 | 2.73 | 1.63 | >100 |
| 104 | 12.2 | 12.48 | 1.43 | >100 |
| 105 | 1.11 | 22.58 | 6.12 | >100 |
| 106 | 11.2 | 9.2 | 20.2 | >100 |
| 107 | 0.60 | >100 | 9.85 | >100 |
| 108 | 0.50 | 2.38 | 1.63 | >100 |
| 109 | 11.2 | 50.0 | 57.8 | >100 |
| 110 | 17.2 | 49.0 | 42.3 | >100 |
| 111 | 11.6 | 19.7 | 24.6 | >100 |
| 112 | 0.57 | 7.7 | 3.7 | 27.3 |
| 113 | 0.57 | 7.1 | 10.0 | 73.4 |
| 114 | 2.42 | 33.8 | 33.0 | >100 |
| 115 | 0.13 | 2.5 | 1.64 | 17.6 |
| 116 | 2.3 | >100 | >100 | >100 |
| 117 | 5.2 | 87.8 | 67.7 | >100 |
| 118 | 0.54 | 0.78 | 0.89 | 12.9 |
| 119 | 0.52 | 33.0 | 18.1 | 4.6 |
| 120 | 0.69 | 26.0 | 13.0 | >100 |
| 121 | 0.09 | 28.0 | 6.8 | 61.0 |
| 122 | 1.8 | 52.0 | 33.0 | >100 |
| 123 | 0.007 | 23.0 | 1.8 | 3.8 |
| 124 | 0.009 | 11.3 | 3.3 | 4.6 |
| 125 | 0.005 | 5.3 | 4.5 | 45.3 |
| 126 | 0.59 | 39.0 | 12.2 | 85.2 |
| 127 | 0.09 | 25.1 | 7.9 | 29.6 |
| 128 | 0.004 | 4.8 | 2.9 | 6.5 |
| 129 | 0.07 | 8.7 | 12.1 | 26.4 |
| 130 | 0.6 | 48.4 | 56.2 | >100 |
| 131 | 0.07 | 21.0 | 9.9 | 24.7 |
| 132 | 0.06 | 46.3 | 56.1 | 2.4 |
| 133 | 0.08 | 23.9 | 17.9 | 16.0 |
| 134 | 0.07 | 17.5 | 25.6 | 4.9 |
| 135 | 0.16 | 45.4 | 34.9 | 4.03 |
| 136 | 0.06 | 23.9 | 29.8 | 4.4 |
| 137 | 0.05 | 15.4 | 24.8 | 9.7 |
| 138 | 0.004 | 4.2 | 5.9 | 25.9 |
| 139 | 0.005 | 6.8 | 8.6 | 19.9 |
| 140 | 0.007 | 9.8 | 10.9 | 34.5 |
| 141 | 0.05 | 23.9 | 45.6 | 23.4 |
| 142 | 0.06 | 34.0 | 43.5 | 61.3 |
| 143 | 0.03 | 25.8 | 34.9 | 34.8 |
| 144 | 0.06 | 23.3 | 33.0 | 46.6 |
| 145 | 0.04 | 34.2 | 34.8 | 45.1 |
| 146 | 0.009 | 23.0 | 32.8 | 24.6 |
| 147 | 0.02 | 13.2 | 26.8 | 15.7 |
| 148 | 0.09 | 23.6 | 34.6 | 44.1 |
| 149 | 0.33 | 67.4 | 75.3 | >100 |
| 150 | 0.46 | 34.4 | 45.9 | 97.8 |
| 151 | 0.12 | 56.8 | 78.8 | 89.6 |

In addition, compounds (I), (II), (III), and (IV) of the present invention have good inhibition activity against the proliferation of other cancer cells, including non-small cell lung cancer cell (H460, H1299), liver tumor cell (HepG2), rectal cancer cell (HT29), leukemia cell (CCRF-CEM), prostate cancer cell (PC-3), ovarian carcinoma cell (SKOV-3), melanoma cell (A375), myeloma cell (RPMI-8226), esophageal carcinoma cell (TE-1), pancreatic cancer cell (CFPAC-1, PANC-1, SW1990, ASPC-1, and BXPC-3), bladder cancer cell (5637), and gastric cancer cell (KATO-III). Results are as follows:

| | Compound | Compound | Compound | Compound |
|---|---|---|---|---|
| IC₅₀ | 123 | 124 | 125 | 128 |
| H460 | 0.22 | 0.34 | 0.16 | 0.14 |
| H1299 | 0.43 | 0.45 | 0.26 | 0.17 |
| HepG2 | 0.01 | 0.02 | 0.009 | 0.02 |
| HT29 | 6.7 | 7.9 | 5.4 | 9.8 |
| CCRF-CEM | 0.12 | 0.22 | 0.09 | 0.53 |
| PC-3 | 13.5 | 23.1 | 26.5 | 8.8 |
| SKOV-3 | 0.09 | 0.10 | 0.09 | 0.08 |
| A375 | 4.65 | 5.65 | 2.21 | 1.66 |
| RPMI-8226 | 90.9 | 99.2 | 89.0 | 78.9 |
| TE-1 | 0.07 | 0.08 | 0.06 | 0.009 |
| CFPAC-1 | 0.007 | 0.009 | 0.006 | 0.008 |
| PANC-1 | 0.76 | 0.89 | 0.97 | 0.1 |
| SW1990 | 0.008 | 0.009 | 0.009 | 0.009 |
| ASPC-1 | 0.007 | 0.008 | 0.005 | 0.008 |
| BXPC-3 | 0.004 | 0.006 | 0.003 | 0.006 |
| 5637 | 0.05 | 0.06 | 0.04 | 0.01 |
| KATO-III | 0.01 | 0.02 | 0.009 | 0.009 |

### Test Example 2. In Vivo Inhibition Test of Cancer Cell Proliferation

Test method: 3×10⁷ CFPAC-1 pancreatic cancer cells were transplanted to subcutaneous tissues of armpit of a Balb/c naked mouse; administration was conducted when the tumor grew to 50-100 mm³, 100-200 mm³, and greater than 200 mm³; the mouse was administered once every other three days, 7 times in total, and 7 mg/kg each time. Normal saline was injected in the control group. Tumor length and diameter were measured by calipers and weight of the moue was weighed until the next day of the final administration. Gross tumor volume was calculated by the following equation:

gross tumor volume (mm³)=½×[short diameter (mm)]²×long diameter (mm)

Test result: in this test, compounds (III) and (IV) of the present invention inhibit cancer cell proliferation well; for example, Examples 123, 124, 125, and 128 show 100% proliferation inhibition activity at an administration dosage of 7 mg/kg compared with the positive control group; compounds 121, 132, 137, 138, 139, 140, 143, 144, 145, 146, and 147 have inhibition ratios of being up to 80% above, and are dose-dependent obviously.

The compounds of the present invention also show good an inhibition effect on cancer cell proliferation after being transplanted into animal models with other cancer cells (non-small cell lung cancer A549).

### Test Example 3. Compound Toxicity Test

Test method: the compounds 121, 123, 124, 125, 128, 132, 137, 138, 139, 140, 143, 144, 145, 146, and 147 in the examples of the present invention were administered every day via tail intravenous injection at a dose of 5 mg/kg for 7 consecutive days, and then body weight changes of the balb/c mice were monitored.

Test result: the mice are in good condition without obviously reduced weight, but gemcitabine mice in the positive control group have obviously reduced weight and are dispirited.

### Test Example 4. Antiviral Activity

Experimental method: 4.59 mg of the compound 125 were weighed and put to a 15 mL centrifugal tube with the addition of 10 mL of sterile water, and dissolved via vortex oscillation until there was no obvious yellow particle, then subpackaged and stored at −20° C., 200 μL/piece (concentration: 1 mM); 2.79 mg Cidofovir (molecular weight: 279) were weighed and added to the 15 mL centrifugal tube, dissolved with the addition of 10 mL sterile water, then subpackaged and stored at −20° C., 200 μL/piece (concentration: 1 mM).

Plaque test:
1. 293A cells were paved into a 24-well plate with 150,000 cells per well, and cultured at 37° C. for 12-24 h, then used for test after growing to 90%.
2. 2% low-melting point agarose gel was melted and put to a 56° C. water bath kettle for further use.
3. Adenovirus having a titer of 10⁹ TCID⁵⁰/mL was diluted by 10⁵ folds; medium in the 24-well plate was removed, and 200 μL of adenoviral diluent was added per well and incubated in a 37° C. 5% CO₂ incubator, shaken once every 30 min, and incubated for 1 h in total.
4. The drug compound 125 to be tested and Cidofovir were double diluted with 2×MEM (16 μL compound 125 was added to 8 mL of 2×MEM, oscillated and mixed well, then 4 mL mixed solution was taken and added to fresh 4 mL of 2×MEM and mixed well, and 5 dilutions were conducted, being 2 μM, 1 μM, 500 nM, 250 nM, and 125 nM, respectively; the maximum concentration of the compound 125 was 1 μM. Similarly, 400 μL Cidofovir was added to 7.6 mL of 2×MEM, oscillated and mixed well, then 4 mL mixed solution was taken and added to fresh 4 mL of 2×MEM and mixed well, and 5 dilutions were conducted, being 50 μM, 25 μM, 12.5 μM, 6.25 μM, and 3.125 μM, respectively); the diluted solution was put at 37° C. for further use.
4. The viral solution was discarded, and agarose gel preheated at 56° C. was added to 2×MEM by the same volume and mixed well, and then 2 mL of mixed solution was added per well, and incubated at room temperature for 30 min (gel).
5. The incubation was conducted in a 37° C. incubator, and during the incubation, a fluorescent microscope was used to observe the replication of virus.

Experimental result: 4 days later, lots of blank plaques formed by non-CPE could be observed in the Cidofovir group; 5 days later, non-CPE blank plaque also appeared in the low concentration group of the Compound 127. The Compound 125 has an inhibiting effect on viral replication, and is very obviously dose-dependent.

Compared with the prior art, the present invention has the following beneficial effects:
1. In the present invention, the quinone-fused imidazole framework is modified systematically to obtain a series of quinonoxime imidazole derivatives and quinonoxime quinone imidazole derivatives having novel structures.
2. The present invention adopts exploratory novel preparation methods; synthesis is simpler; raw materials are easier to get and low in cost; moreover, the present invention is suitable for large-scale preparation.

Detailed examples of the present invention are described above. It need be understood that the present invention is not limited to the above specific embodiments. Those skilled in the art would make various transformations or amendments within the scope of the claims, which will affect the substantive content of the present invention.

What is claimed is:

1. A fused quinonoxime imidazolium derivative, having a structure represented by the following Formula (I) or (II): 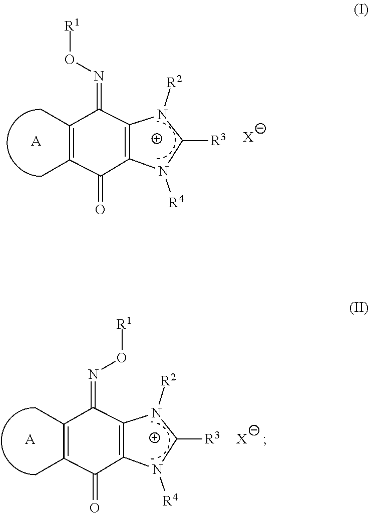
wherein,
a ring A is selected from an aromatic ring having zero, one, or more substituents, or a heteroaromatic ring having zero, one, or more substituents;
R¹, R², R³, and R⁴ are identical or different, and each of the R¹, the R², the R³, and the R⁴ represents-hydrogen atom, -lower linear/branched alkyl having zero, one, or more substituents, -lower alkenyl having zero, one, or more substituents, -lower alkynyl having zero, one, or more substituents, -saturated heterocyclyl having zero, one, or more substituents, -cycloalkyl having zero, one, or more substituents, -cycloalkenyl having zero, one, or more substituents, -aryl having zero, one, or more substituents, and -heteroaryl having zero, one, or more substituents;
substituents on the ring A as well as in the R¹, the R², the R³, and the R⁴ are selected from a radical of group B;
the group B: -lower linear/branched alkyl having zero, one, or more substituents, -lower alkenyl having zero, one, or more substituents, -lower alkynyl having zero, one, or more substituents, -saturated heterocyclyl having zero, one, or more substituents, -cycloalkyl having zero, one, or more substituents, -cycloalkenyl having zero, one, or more substituents, -aryl having zero, one, or more substituents, -heteroaryl having zero, one, or more substituents, —OR^{a}, —SR^{a}, —O-lower alkylene-OR^{a}, —O-lower alkylene-O-lower alkylene-OR^{a}, —O-lower alkylene-O-lower alkylene-O-lower alkylene-OR^{a}, —O-lower alkylene-NR^{a}R^{b}, —O-lower alkylene-O-lower alkylene-NR^{a}R^{b}, —O-lower alkylene-NR^{c}-lower alkylene-NR^{a}R^{b}, —O—CO—NR^{a}R^{b}, —SOR^{a}, —SO₂R^{a}, —SO₂NR^{a}R^{b}, —NR^{a}—SO₂R^{b}, —NR^{a}R^{b}, —NR^{c}-lower alkylene-NR^{a}R^{b}, —N(-lower alkylene-NR^{a}R^{b})₂, —NO₂, —CN, -halogen, —CO₂R^{a}, —COO; —CONR^{a}R^{b}, —CONR^{a}—O—R^{b}, —NR^{a}—COR^{b}, —NR^{a}—CO—NR^{b}R^{c}, —OCOR^{a}, and —COR^{a};
R^{a}, R^{b}, and R^{c} are identical or different, and each of the R^{a}, the R^{b}, and the R^{c} represents-hydrogen atom, -lower linear/branched alkyl having zero, one, or more substituents, -lower alkenyl having zero, one, or more substituents, -lower alkynyl having zero, one, or more substituents, -saturated heterocyclyl having zero, one, or more substituents, -cycloalkyl having zero, one, or more substituents, -cycloalkenyl having zero, one, or more substituents, -aryl having zero, one, or more substituents, -heteroaryl having zero, one, or more substituents, -lower alkylene-(5 to 7-membered saturated heterocycle having zero, one, or more substituents), -lower alkylene-(cycloalkyl having zero, one, or more substituents), -lower alkylene-(aryl having zero, one, or more substituents), and -lower alkylene-(heteroaryl having zero, one, or more substituents), wherein the substituents refer to lower alkyl or heteroalkyl;
X⁻ is a coanion comprising a halide ion, a sulfonate ion, an acetate ion, a trifluoroacetate ion, a carbonate ion, and a sulfate ion; and when an anion on a substituent and an imidazolium cation form an inner salt, X⁻ is absent.

2. The fused quinonoxime imidazolium derivative according to claim 1, wherein in the Formula (I) or (II),
the ring A is selected from a benzene ring, a naphthalene ring, or a NO₂-substituted benzene ring;
the R¹ is selected from -hydrogen atom, -lower alkyl, -lower alkynyl, -lower alkenyl, -lower linear/branched alkyl-O-lower linear/branched alkyl, -lower linear/branched alkyl-O-aryl, -lower linear/branched alkyl-O-lower linear/branched alkyl-O-lower linear/branched alkyl, -lower linear/branched alkyl-O-lower linear/branched alkyl-O-lower linear/branched alkyl-O-lower linear/branched alkyl, -aryl, -(5 to 7-membered) saturated heterocycle, and -heteroaryl;
the R² is selected from lower linear/branched alkyl;
the R³ is selected from -hydrogen atom, -lower linear/branched alkyl, -(3 to 7-membered) saturated cycloalkyl, -heterocyclyl, -aryl, -aryl substituted by lower linear/branched alkyl, -aryl substituted by halogen, -heteroaryl, -heteroaryl substituted by lower linear/branched alkyl, —O-lower linear/branched alkyl, —S-lower linear/branched alkyl, -acyl, and -acyl substituted by lower alkyl; and
the R⁴ is selected from -hydrogen atom, -lower linear/branched alkyl, -lower alkenyl, -lower alkynyl, -aryl, —CN, -(3 to 7-membered) saturated cycloalkyl, -aryl substituted by halogen, -aryl substituted by lower alkyl, -heteroaryl, —CO₂R^{a}, -saturated heterocyclyl, -lower linear/branched alkyl-OR^{a}, -lower linear/branched alkyl-O-lower linear/branched alkyl, -lower linear/branched alkyl-S-lower linear/branched alkyl, and -lower linear/branched alkyl-O-lower linear/branched alkyl-OR^{a}, wherein the R^{a} is -lower linear/branched alkyl.

3. A fused quinonoxime imidazole derivative and a hydrochloride thereof, having a structure represented by the following Formula (III) or (IV): 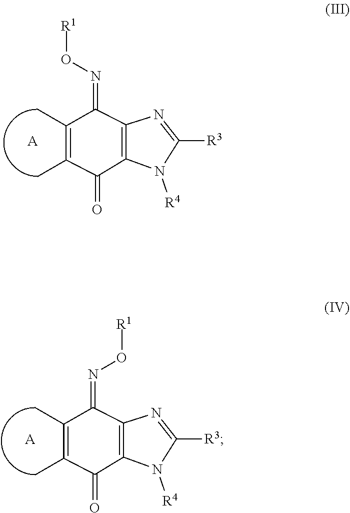
wherein,
a ring A is selected from an aromatic ring having zero, one, or more substituents, or a heteroaromatic ring having zero, one, or more substituents;
R¹, R³, and R⁴ are identical or different, and each of the R¹, the R³, and the R⁴ represents-hydrogen atom, -lower linear/branched alkyl having zero, one, or more substituents, -lower alkenyl having zero, one, or more substituents, -lower alkynyl having zero, one, or more substituents, -saturated heterocyclyl having zero, one, or more substituents, -cycloalkyl having zero, one, or more substituents, -cycloalkenyl having zero, one, or more substituents, -aryl having zero, one, or more substituents, and -heteroaryl having zero, one, or more substituents;
substituents on the ring A as well as in the R¹, the R³, and the R⁴ are selected from a radical in group B;
the group B: -lower linear/branched alkyl having zero, one, or more substituents, -lower alkenyl having zero, one, or more substituents, -lower alkynyl having zero, one, or more substituents, -saturated heterocyclyl having zero, one, or more substituents, -cycloalkyl having zero, one, or more substituents, -cycloalkenyl having zero, one, or more substituents, -aryl having zero, one, or more substituents, -heteroaryl having zero, one, or more substituents, —OR^{a}, —SR^{a}, —O-lower alkylene-OR^{a}, —O-lower alkylene-O-lower alkylene-OR^{a}, —O-lower alkylene-O-lower alkylene-O-lower alkylene-OR^{a}, —O-lower alkylene-NR^{a}R^{b}, —O-lower alkylene-O-lower alkylene-NR^{a}R^{b}, —O-lower alkylene-NR^{c}-lower alkylene-NR^{a}R^{b}, —O—CO—NR^{a}R^{b}, —SOR^{a}, —SO₂R^{a}, —SO₂NR^{a}R^{b}, —NR^{a}—SO₂R^{b}, —NR^{a}R^{b}, —NR^{c}-lower alkylene-NR^{a}R^{b}, —N(-lower alkylene-NR^{a}R^{b})₂, —NO₂, —CN, -halogen, —CO₂R^{a}, —COO—, —CONR^{a}R^{b}, —CONR^{a}—O—R^{b}, —NR^{a}—COR^{b}, —NR^{a}—CO—NR^{b}R^{c}, —OCOR^{a}, and —COR^{a};
R^{a}, R^{b}, and R^{c} are identical or different, and each of the R^{a}, the R^{b}, and the R^{c} represents-hydrogen atom, -lower linear/branched alkyl having zero, one, or more substituents, -lower alkenyl having zero, one, or more substituents, -lower alkynyl having zero, one, or more substituents, -saturated heterocyclyl having zero, one, or more substituents, -cycloalkyl having zero, one, or more substituents, -cycloalkenyl having zero, one, or more substituents, -aryl having zero, one, or more substituents, and -heteroaryl having zero, one, or more substituents; wherein the substituents refer to lower alkyl or heteroalkyl.

4. The fused quinonoxime imidazole derivative and the hydrochloride thereof according to claim 3, wherein in the Formula (III) or (IV),
the ring A is selected from a -benzene ring, a -naphthalene ring, or a NO₂-substituted benzene ring;
the R¹ is selected from -hydrogen atom, -lower alkyl, -lower alkynyl, -lower alkenyl, -lower linear/branched alkyl-O-lower linear/branched alkyl, -lower linear/branched alkyl-O-aryl, -lower linear/branched alkyl-O-lower linear/branched alkyl-O-lower linear/branched alkyl, -lower linear/branched alkyl-O-lower linear/branched alkyl-O-lower linear/branched alkyl-O-lower linear/branched alkyl, -aryl, -(5 to 7-membered) saturated heterocycle, and -heteroaryl;
the R³ is selected from -hydrogen atom, -lower linear/branched alkyl, -(3 to 7-membered) saturated cycloalkyl, -heterocyclyl, -aryl, -aryl substituted by lower linear/branched alkyl, -aryl substituted by halogen, -heteroaryl, -heteroaryl substituted by lower linear/branched alkyl, —O-lower linear/branched alkyl, —S-lower linear/branched alkyl, -acyl, and acyl substituted by lower alkyl; and
the R⁴ is selected from -hydrogen atom, -lower linear/branched alkyl, -lower alkenyl, -lower alkynyl, -aryl, —CN, -(3 to 7-membered) saturated cycloalkyl, -aryl substituted by halogen, -aryl substituted by lower alkyl, -heteroaryl, —CO₂R^{a}, -saturated heterocyclyl, -lower linear/branched alkyl-OR^{a}, -lower linear/branched alkyl-O-lower linear/branched alkyl, -lower linear/branched alkyl-S-lower linear/branched alkyl, and -lower linear/branched alkyl-O-lower linear/branched alkyl-OR^{a}, wherein the R^{a} is lower linear/branched alkyl.

5. A pharmaceutical composition, comprising one or more of the fused quinonoxime imidazolium derivative according to claim 2, and a pharmaceutically acceptable carrier.

6. A pharmaceutical composition, comprising one or more of the fused quinonoxime imidazole derivative according to claim 4, or the hydrochloride thereof, and a pharmaceutically acceptable carrier.

7. (canceled)

8. A preparation method of the fused quinonoxime imidazolium derivative according to claim 2, comprising the following steps:
A, serving a protic solvent as a solvent, and subjecting a substituted quinone imidazole derivative 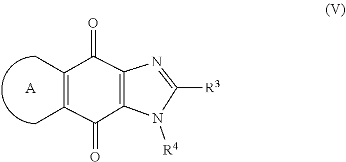
to an oximation reaction with R¹ONH₂·HCl under a catalytic action of a weak base or with corresponding R¹ONH₂ under an action of a weak-base hydrochloride, to obtain fused quinonoxime imidazole derivatives 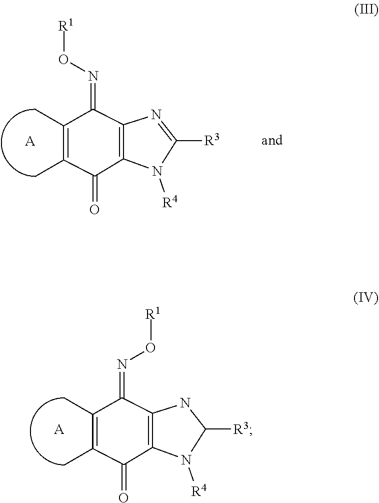
and
B, performing an N-alkylation reaction on the fused quinonoxime imidazole derivative (III) or (IV) and a corresponding halide reagent R²X in an organic solvent, to obtain the fused quinonoxime imidazolium derivative (I) or (II).

9. The preparation method according to claim 8, wherein in the step A, a molar ratio of the substituted quinone imidazole derivative 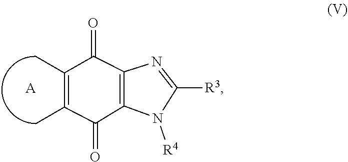
to the R¹ONH₂·HCl, and to the weak base is 1:(1.2-5):(0.001-0.01); the protic solvent is selected from methanol, ethanol, and isopropanol; the weak base is selected from pyridine, triethylamine, and potassium carbonate; and the oximation reaction is performed at 90-125° C. for 12-48 h.

10. The preparation method according to claim 8, wherein in the step B, a molar ratio of the fused quinonoxime imidazole derivative (III) or (IV) to the corresponding halide reagent R²X is (1:1)-(1:100); the organic solvent is selected from acetonitrile, ethyl acetate, and tetrahydrofuran; and the N-alkylation reaction is performed at 50-120° C. for 8-48 h.

11. A preparation method of the fused quinonoxime imidazole derivative according to claim 4, comprising the following steps:
A, serving a protic solvent as a solvent, and subjecting a substituted quinone imidazole derivative 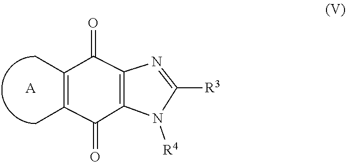
to an oximation reaction with R¹ONH₂·HCl under a catalytic action of a weak base or with corresponding R¹ONH₂ under an action of a weak-base hydrochloride, to obtain the fused quinonoxime imidazole derivatives

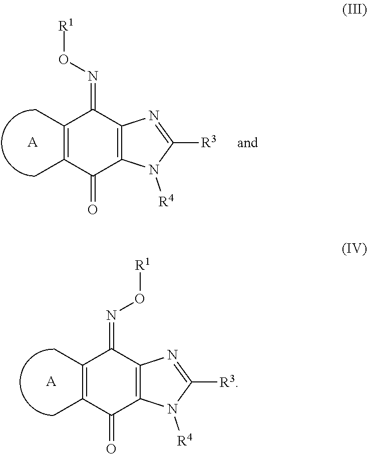

12. The preparation method according to claim 11, wherein in the step A, a molar ratio of the substituted quinone imidazole derivative 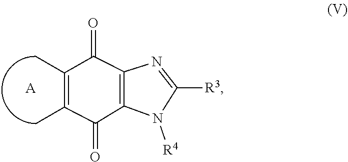
to the R¹ONH₂·HCl, and to the weak base is 1:(1.2-5):(0.001-0.01); the protic solvent is selected from methanol, ethanol, and isopropanol; the weak base is selected from pyridine, triethylamine, and potassium carbonate; and the oximation reaction is performed at 90-125° C. for 12-48 h.
